(19) United States
(12) Patent Application Publication
(10) Pub. No.: US 2010/0041121 A1
(43) Pub. Date: Feb. 18, 2010

(54) METABOLICALLY ENGINEERED ORGANISMS FOR THE PRODUCTION OF HYDROGEN AND HYDROGENASE

(75) Inventor: Guangyi Wang, Honolulu, HI (US)

Correspondence Address: KNOBBE MARTENS OLSON & BEAR LLP, 2040 MAIN STREET, FOURTEENTH FLOOR, IRVINE, CA, 92614, US

(73) Assignee: UNIVERSITY OF HAWAII, Honolulu, HI (US)

(21) Appl. No.: 12/160,764

(22) PCT Filed: Feb. 1, 2007
(86) PCT No.: PCT/US2007/002778

§ 371 Date: Nov. 3, 2009

Related U.S. Application Data
(60) Provisional application No. 60/764,519, filed on Feb. 1, 2006.

(51) Int. Cl. C12N 15/70; C12N 1/21; C12N 9/02; C12P 3/00

(52) U.S. Cl. 435/168; 435/320.1; 435/252.33; 435/189

(57) ABSTRACT

The present application relates to the use of metabolically-engineered microbial cells for the production of hydrogen and hydrogenase enzymes. The microbial cells are strains of E. coli which are genetically engineered to optimize the cell for production of hydrogen or active hydrogenase. The strains of E. coli are transformed with at least one expression vector directed towards the biosynthesis of a hydrogenase enzyme. Methods of hydrogen production, fuel-cell systems and recombinant fuel-cell catalysts are also provided.

### CROSS-REFERENCE TO RELATED APPLICATIONS

The application present application claims priority to U.S. Provisional Application No. 60/764,519, filed Feb. 1, 2006, which is hereby incorporated by reference in its entirety.

### STATEMENT REGARDING FEDERALLY SPONSORED R&D

Certain aspects of the invention disclosed herein were made with United States government support under the U.S. Department of Energy Grant No. DE-FG02-06ER15770. The United States government has certain rights in these aspects of the invention.

### BACKGROUND OF THE INVENTION

1. Field of the Invention

This invention relates to the field of enzymes for use in hydrogen-based fuel cells. More specifically, the invention relates to recombinantly-expressed hydrogenases and their applications for use in hydrogen-based fuel cells as well as production of hydrogen in engineered E. coli strains.

2. Description of the Related Art

There has been an increasing interest in alternative fuels due to rising petroleum costs, escalating diplomatic tensions with oil producing countries, and the rising levels of greenhouse gases in the atmosphere (Kessel, D. G. 2000. J. Pet. Sci. Eng. 26:157-168, which is incorporated herein by reference in its entirety). As a solution to these problems, hydrogen is an ideal fuel due to its high energy content and nonpolluting nature (Lin, C. Y., and C. H. Lay. 2004. Int. J. Hydrog. Energy 29:275-281; Oh, S. E. et al. 2003. Environ. Sci. Technol. 37:5186-5190, each of which is incorporated herein by reference in its entirety). The key to a hydrogen economy is finding an efficient, inexpensive, and renewable process for the production of hydrogen (Hallenbeck, P. C., and J. R. Benemann. 2002. Int. J. Hydrog. Energy 27:1185-1193, which is incorporated herein by reference in its entirety) while also achieving the equally important goal of economically converting hydrogen into usable energy (Karyakin, A. A. et al. 2005. Biochem. Soc. Transact. 33:73-75, which is incorporated herein by reference in its entirety).

Platinum is the most commonly used catalyst for fuel cells. Currently, platinum catalysts are used to split hydrogen into two protons and two electrons. However, it is expensive and with limited availability, contributes greatly to the cost of fuel cells. Platinum is also easily poisoned by impurities, such as carbon monoxide (CO) and sulfur (S), which are commonly present in industrial hydrogen (Karyakin, A. A. et al. 2002. Electrochem. Commun. 4:417-420, which is incorporated herein by reference in its entirety).

Several hydrogenases have been proposed as candidates for a good bioelectrocatalyst in hydrogen oxidation that can potentially hold promise as an inexpensive alternative to platinum in fuel cell development (Jones, A. K. et al. Chem. Commun. 21:866-867; Morozov, S. V. et al. 2002. Bioelectrochemistry 55:169-171; Vincent, K. A. et al. 2005. Proc. Natl. Acad. Sci. USA 102:16951-16954, each of which is incorporated herein by reference in its entirety). For example, the [NiFe] hydrogenase from the purple bacterium Allochromatium vinosum is a remarkably active electrocatalyst. The electrode coated with the hydrogenase catalyzes hydrogen oxidation with a turnover number exceeding 1500 sec⁻¹ under a partial pressure of 0.1 bar of H₂ at 30° C. (Pershad, H. R. et al. 1999. Biochemistry 38:8992-8999, which is incorporated herein by reference in its entirety). The oxidative activity of the active site is comparable to that of a platinum fuel cell catalyst (Jones, A. K. et al. 2002. supra), but unlike the costly platinum electrode, the hydrogenase-coated electrode is less susceptible to CO poisoning. Finally, hydrogenase confers fuel specificity and greater turnover rates than their metal counterparts, and its use in place of metal catalysts will allow fuel cells to be operated at neutral pH and ambient temperatures, which are the conditions much more favorable for the handling of fuel cells (Ikeda, T., and K. Kano. 2001. J. Biosci. Bioeng. 92:9-18, which is incorporated herein by reference in its entirety; Karyakin, A. A. 2002. supra). Therefore, the [NiFe]-hydrogenases are a promising electrocatalyst in fuel cells, offering an alternative to platinum.

Though [NiFe]-hydrogenases exhibit promise, there remain problems associated with use of these and other hydrogenase enzymes. The stability of hydrogenases has been one of the major disadvantages in their use in enzyme fuel cells (Sasaki, S., and I. Karube. 1999. Trends Biotechnol. 17:50-52, which is incorporated herein by reference in its entirety). Furthermore, though the enzymes demonstrate less susceptibility to CO poisoning than does platinum, commercial use requires further improvement in terms of both the sensitivity to CO as well as to oxygen. In addition, the lack of hydrogenase availability in large quantities limits their potential application in enzyme fuel cells (Kessel, D. G. 2000. supra). Therefore, production of stable hydrogenase in large quantities and with desired catalytic properties will greatly enhance the application of this interesting bioelectrocatalyst for hydrogen fuel.

### SUMMARY OF THE INVENTION

The invention relates generally to expression vectors, microorganisms, methods and reactor systems to produce hydrogen and active hydrogenase enzymes for energy- and electricity-generating applications. The expression vectors and microorganism can be used in fermentation methods to produce the products of interest. Both the hydrogen and active hydrogenase products can be incorporated into a system such as, for example, a fuel cell system for producing electricity from hydrogen.

In various embodiments of the invention, an expression vector comprising one or more nucleic acid sequences associated with biosynthesis of a hydrogenase enzyme, wherein expression of the vector within a predetermined host results in altered hydrogenase activity that is different from native hydrogenase activity within the host, is provided. The expression vector can comprise one or more nucleic acid sequences encoding a hydrogenase enzyme or a fragment thereof. The altered hydrogenase activity can comprise elevated total hydrogenase activity within the host. In some embodiments, the altered hydrogenase activity comprises a hydrogenase activity with at least one distinct property as compared with the native hydrogenase activity within the host, wherein the distinct property is selected from: increased enzyme yield, increased specific activity, improved temperature-independent stability, improved pH-independent stability, increased catalytic efficiency, increased hydrogen evolution rate. In some embodiments, the altered hydrogenase activity is associated with a condition selected from the group consisting of: increased enzyme yield, improved expression levels of hydrogenase, improved specific activity, improved temperature-independent stability, improved pH-independent stability, increased catalytic efficiency, increased hydrogen evolution rate, improved host compatibility, elevated cofactor levels, light energy-dependent ATP production.

In various embodiments of the invention, the predetermined host is selected from E. coli strains: GW12, GW12A, GW1234, GW12AP, GW1234P, GW12APN, GW1234PN, GW0123HE and GW0123HEP.

In various embodiments of the invention, the expression vector comprises a nucleic acid sequence derived from a microbial species selected from: R. eutropha, E. coli, and C. acetobutylicum. In some embodiments, the expression vector comprises a gene or gene fragment, or a derivative thereof, selected from: hoxBC, hoxFUYH, hoxKGZ, hycEG, hydAEFG, hyp_{RE}, hoxMLOQRTV, and hoxWI. In further embodiments, the expression vector comprises at least one sequence selected from the group: SEQ ID NO: 62, 66, 72, 73, 74, 75, 76 and 81.

In various embodiments of the invention, an expression vector for the expression of an uptake hydrogenase enzyme is provided. The vectors comprise one or more nucleic acid sequences encoding an uptake hydrogenase enzyme, an uptake hydrogenase enzyme accessory gene and fragments thereof.

The uptake hydrogenase enzyme expressed by the vector is preferably a hydrogenase enzyme from R. eutropha. The hydrogenase enzyme from R. eutropha can be, for example, regulatory hydrogenase, membrane-bound hydrogenase, or soluble hydrogenase. In some embodiments, the hydrogenase enzyme is regulatory hydrogenase. In some embodiments, the hydrogenase enzyme is membrane-bound hydrogenase. In some embodiments, the hydrogenase enzyme is soluble hydrogenase. In addition, the vector can comprise one or more nucleic acid sequences selected from the group: SEQ ID NO: 72, 73 and 74. In some embodiments, the vector comprises SEQ ID NO:72. In some embodiments, the vector comprises SEQ ID NO: 73. In some embodiments, the vector comprises SEQ ID NO: 74.

The uptake hydrogenase enzyme accessory gene of the vector is preferably involved in the maturation and expression of active uptake hydrogenase enzyme. The accessory gene can comprise one or more of the following: hyp_{RE}, hoxMLOQRTV, or hoxWI. In some embodiments, the accessory gene comprises hyp_{RE}. In some embodiments, the accessory gene comprises hoxMLOQRTV. In some embodiments, the accessory gene comprises hoxWI. In addition, the vector can comprise one or more nucleic acid sequences selected from the group: SEQ ID NO: 66, 75 and 76. In some embodiments, the vector comprises SEQ ID NO: 66. In some embodiments, the vector comprises SEQ ID NO: 75. In some embodiments, the vector comprises SEQ ID NO: 76.

In various embodiments of the invention, a vector for the expression of a hydrogenase enzyme that catalyzes a reaction to evolve hydrogen is provided. The vector can comprise one or more nucleic acid sequences associated with biosynthesis of a hydrogenase enzyme selected from the group: hydrogenase 3 from E. coli, Fe-hydrogenase from C. acetobutylicum. In some embodiments, the vector comprises a nucleic acid that encodes hydrogenase 3 from E. coli. In some embodiments, the vector comprises a nucleic acid that encodes Fe-hydrogenase from C. acetobutylicum. In some embodiments, the vector comprises nucleic acids that encode hydrogenase 3 from E. coli and Fe-hydrogenase from C. acetobutylicum. Furthermore, the vector can comprise at least one nucleic acid sequence selected from the group: SEQ ID NO: 62 and 81. In some embodiments, the vector comprises SEQ ID NO: 62. In some embodiments, the vector comprises SEQ ID NO: 81. In some embodiments, the vector comprises SEQ ID NO: 62 and 81.

In various embodiments of the invention, a hydrogenase-null microorganism for heterologous expression of active hydrogenase is provided. The hydrogenase-null microorganism can be, for example, E. coli. In some embodiments, the hydrogenase-null microorganism is transformed with an expression vector comprising one or more nucleic acid sequences associated with biosynthesis of a hydrogenase enzyme, wherein expression of the vector within a predetermined host results in altered hydrogenase activity that is different from native hydrogenase activity within the host.

In various embodiments of the invention, there is provided an E. coli microorganism, wherein a portion of the genome is deleted, and wherein said portion comprises at least one sequence selected from: SEQ ID NO: 56, 57, 61, 63-65.

In various embodiments of the invention, there is provided an E. coli microorganism wherein one or more sequences associated with a hydrogenase enzyme is deleted from the host genome. The hydrogenase enzyme can be, for example, hydrogenase 1, hydrogenase 2, hydrogenase 3 or hydrogenase 4. In some embodiments, one sequence is deleted from the genome. In some embodiments, two sequences are deleted from the genome. In some embodiments, three or more sequences are deleted from the genome. In some embodiments, a sequence associated with hydrogenase 1 is deleted from the genome. In some embodiments, a sequence associated with hydrogenase 2 is deleted from the genome. In some embodiments, sequences associated with hydrogenase 1 and hydrogenase 2 are deleted from the genome. In some embodiments, a sequence associated with hydrogenase 3 is deleted from the genome. In some embodiments, a sequence associated with hydrogenase 4 is deleted from the genome. In some embodiments, sequences associated with hydrogenase 1, hydrogenase 2 and hydrogenase 3 are deleted from the genome. In some embodiments, sequences associated with hydrogenase 1, hydrogenase 2, hydrogenase 3 and hydrogenase 4 are deleted from the genome.

In various embodiments of the invention, there is provided an E. coli microorganism wherein at least one sequence associated with a hydrogenase enzyme is deleted from the host genome, wherein the at least one sequence is selected from: SEQ ID NO: 56, 57, 61 and 63-65. In some embodiments, one sequence is deleted from the genome. In some embodiments, two sequences are deleted from the genome. In some embodiments, three or more sequences are deleted from the genome. In some embodiments, SEQ ID NO: 56 is deleted from the genome. In some embodiments, SEQ ID NO: 57 is deleted from the genome. In some embodiments, SEQ ID NO: 56 and 57 are deleted from the genome. In some embodiments, SEQ ID NO: 56, 57 and 61 are deleted from the genome. In some embodiments, SEQ ID NO: 63 is deleted from the genome. In some embodiments, SEQ ID NO: 64 is deleted from the genome. In some embodiments, SEQ ID NO: 56, 57 and 63 are deleted from the genome. In some embodiments, SEQ ID NO: 56, 57, 63 and 64 are deleted from the genome.

In various embodiments of the invention, a hydrogenase-null microorganism is transformed with one or more expression vectors comprising: SEQ ID NO: 62, 66, 72-76 and 81.

In various embodiments of the invention, a hydrogenase-null microorganism is provided wherein one or more of SEQ ID NO: 62, 70-76, 81 and 82 is integrated into the genome of the microorganism.

In various embodiments of the invention, a host selected from E. coli strains: GW12, GW12A, GW1234, GW12AP, GW1234P, GW12APN, GW1234PN, GW0123HE and HW0123HEP is transformed with one or more expression vectors comprising: SEQ ID NO: 62, 66, 72-76 and 81.

In various embodiments of the invention, a host selected from E. coli strains: GW12, GW12A, GW1234, GW12AP, GW1234P, GW12APN, GW1234PN, GW0123HE and HW0123HEP is provided, wherein one or more of SEQ ID NO: 62, 72-76 and 81 is integrated into the genome of the microorganism.

In various embodiments of the invention, a fuel cell system for oxidation of molecular hydrogen comprising a hydrogenase-null microorganism for heterologous expression of an active hydrogenase, wherein the microorganism is transformed with one or more expression vectors comprising one or more nucleic acid sequences associated with biosynthesis of a hydrogenase enzyme, wherein expression of the at least one vector within a predetermined host results in altered hydrogenase activity that is different from native hydrogenase activity within the host, is provided. The nucleic acid sequences can be associated with uptake hydrogenase enzymes, including, but not limited to: regulatory hydrogenase, membrane-bound hydrogenase and soluble hydrogenase. In some embodiments, the one or more expression vectors comprise a sequence selected from: SEQ ID NO: 66 and 72-76.

In various embodiments of the invention, a recombinant fuel-cell catalyst comprising one or more hydrogenase enzymes selected from: a regulatory hydrogenase, a soluble hydrogenase and a membrane-bound hydrogenase, is provided. The one or more hydrogenase is encoded by one or more of SEQ ID NO: 72, 73 and 74. In some embodiments, the catalyst comprises one or more hydrogenase enzymes produced by expression of an expression vector comprising one or more nucleic acid sequences associated with biosynthesis of a hydrogenase enzyme, wherein expression of the vector within a predetermined host results in altered hydrogenase activity that is different from native hydrogenase activity within the host. The catalyst can also comprise one or more hydrogenase enzymes expressed in a hydrogenase-null microorganism comprising such an expression vector.

In various embodiments of the invention, a method of producing hydrogen is provided. A hydrogenase-null microorganism transformed with an expression vector comprising one or more nucleic acid sequences associated with biosynthesis of a hydrogenase enzyme, wherein expression of the at least one vector within a predetermined host results in altered hydrogenase activity that is different from native hydrogenase activity within the host, is provided. The microorganism is grown in a cell culture medium, followed by recovery of the hydrogen produced by the microorganism.

### BRIEF DESCRIPTION OF THE DRAWINGS

FIG. 1 illustrates the biochemical and enzymatic activities involved in the generation of hydrogen in an engineered E. coli strain.

FIG. 2 is a gel that illustrates the gene knock-out of the large and small subunits of hydrogenase 1, 2, 3, and 4 in engineered E. coli strains.

FIG. 3 is a bar graph that illustrates hydrogen yields among native and engineered E. coli strains.

FIG. 4 is a graph that illustrates the effect of deleting the uptake hydrogenases 1 and 2 on the production of hydrogen in genetically engineered E. coli strains.

FIG. 5 is a bar graph that illustrates enzymatic hydrogenase activity among various genetically engineered E. coli strains.

### DETAILED DESCRIPTION OF THE PREFERRED EMBODIMENT

Biological production of hydrogen is promising due to its reliance on renewable resources such as solar energy and organic wastes. Particularly, dark fermentation of organic substrates under anaerobic conditions seems to hold the best promise for biohydrogen production due to low operating costs, relative high production efficiency, and relatively stable hydrogen evolving enzymes (Das, D., and T. N. Veziroglu. 2001. Int. J. Hydrog. Energy 26:13-28; Hallenbeck, P. C., and J. R. Benemann. 2002. Int. J. Hydrog. Energy 27:1185-1193, each of which is incorporated herein by reference in its entirety). Organic wastes from agriculture or sewage can be fed into anaerobic bioreactors, achieving the dual goals of waste management and hydrogen production (Benemann, J. 1996. Nat. Biotechnol. 14:1101-1103, which is incorporated herein by reference in its entirety; Chittibabu, G. et al. 2006. supra). However, several obstacles such as inhibition of hydrogen-evolving enzymes by oxygen, hydrogen consumption by uptake hydrogenases, overall low production yield, and the gas impurities still exist as main stumbling blocks for the viable macroscale utilization and production of hydrogen from renewable sources (Pinto, F. A. L. et al. 2002. Int. J. Hydrog. Energy 27:1209-1215, which is incorporated herein by reference in its entirety; Das, D., and T. N. Veziroglu. 2001. supra; Hallenbeck, P. C., and J. R. Benemann. 2002. supra).

Hydrogenases have found use in a variety of biotechnological applications, including biohydrogen production, applications in biofuel cells, biosensors, wastewater treatment, the prevention of microbial-induced corrosion, and the generation and regeneration of NADP cofactors (Mertens, R., and A. Liese. 2004. Curr. Opin. Biotechnol. 15:343-348, which is incorporated herein by reference in its entirety). Because of their remarkable electrochemical characteristics, hydrogenases have tremendous potential to be used in hydrogen production and oxidation as a bioelectrocatalyst. First, hydrogenases, such as the Fe-only hydrogenase, have been a key enzyme involved in biohydrogen production and are used as a catalyst in photoinduced hydrogen production (Hallenbeck, P. C., and J. R. Benemann. 2002. supra; Qian, D. J. et al. 2002. Int. J. Hydrog. Energy 27:1481-1487, which is incorporated herein by reference in its entirety). Second, hydrogenases, such as the [NiFe] hydrogenase, are good bioelectrocatalysts in hydrogen oxidation and have implications for fuel cell development (Jones, A. K. et al. 2002; Morozov, S. V. et al. 2002. supra).

As herein described, host bacterial strains were engineered in order to produce hydrogenases for use as biocatalysts as well as to produce hydrogen. Four hydrogenases responsible for the utilization and production of hydrogen in Escherichia coli were sequentially deleted from the organism genome using genetic engineering tools. It was demonstrated that engineered strains in which hydrogen-uptake genes were deleted produced much higher levels of hydrogen than the parental strain. In addition, all four hydrogenase genes were deleted in E. coli to engineer a hydrogenase-null host, from which strains for hydrogenase enzyme production and hydrogen evolution were developed.

The hydrogenase-null strain was used to produce functional hydrogenase as a biocatalyst for hydrogen fuel cells and is envisioned as a platform strain for protein engineering of hydrogenases with improved catalytic and stability properties. Using [NiFe] hydrogenase genes obtained from Ralstonia eutropha, the genes for regulatory hydrogenase (RH), membrane-bound hydrogenase (MBH) and soluble hydrogenase (SH) of R. eutropha were successfully introduced and expressed in the hydrogenase-null strain. In addition, accessory (or maturation) genes or proteins were co-expressed in the hydrogenase-null strain to improve expression of the active forms of hydrogenase.

In addition, as herein described, “mixotrophic” strains can be developed from the hydrogenase-null host strain to produce hydrogen through biological means and to optimize the biochemical processes by which hydrogen is evolved. For example, using the hydrogenase-null strain platform, Fe-hydrogenase genes obtained from Clostridium acetobutylicum, is introduced and expressed to increase hydrogen output. Additional developments include the engineering of strains to generate ATP (the energy currency of biological activities) from light energy in order to aid the evolution of hydrogen, which is an endergonic biological process. Furthermore, the strains can be engineered to increase production of cofactors involved in hydrogen production, such as, for example, NAD⁺ and NADH. Strains containing hydrogen-evolution enzymes can be used to produce hydrogen in a fermenter.

### Expression of Hydrogenase in Heterologous Hosts

The development of a heterologous host for the production of hydrogenases can aid in producing large quantities of the enzymes for use in fuel cells and for making improvements in their bioelectrocatalytic properties. Although efforts have been made to express hydrogenase in heterologous hosts, limited progress has previously only been achieved (Casalto, L., and M. Rousset. 2001. Trends Microbiol. 9:228-237; Friedrich, B., and E. Schwartz. 1993. Annu. Rev. Microbiol. 47:351-383; Maier, T., and A. Bock. 1996. In R. P. Hausinger, G. L. Eichhor, and L. C. Marilli (ed.), Mechanism of metallocenter assembly, New York, p. 173-192, each of which is incorporated herein by reference in its entirety). The main obstacle is that assembly of the active hydrogenase requires complicated biological processes, including careful coordination of cofactor biosynthesis and insertion, subunit recruitment, and protein target processes (Paschos, A. et al. 2002. J. Biol. Chem. 277:49945-49951, which is incorporated herein by reference in its entirety; Vignais, P. M. et al. 2001. supra). These processes are involved in the products of three functional classes of genes that encode structural proteins, regulatory proteins, and maturation (or accessory) proteins. All genes encoding bacterial hydrogenase and products involved in the maturation of hydrogenase are clustered, either on chromosome or on a megaplasmid (Vignais, P. M. et al. 2001. supra).

There are two main groups of maturation genes which are differentiated by the phenotypes resulting from their mutation (Casalto, L., and M. Rousset. 2001. supra). The first group of genes is mainly located on the same transcription unit as the structural genes. Disruption of this group of genes specifically impairs the processing or activity of the hydrogenase encoded in cis in the operon without affecting the maturation of other isoenzymes. The maturation processes mediated by the products of this family of accessory genes can not be complemented in trans by homologous genes from the other isoenzyme operons, regardless of the degree of similarity (Bernhard, M. et al. 1996. J. Bacteriol. 178:4522-4529, which is incorporated herein by reference in its entirety; Menon, N. K. et al. 1994; Sauter, M. et al. 1992. supra). This specific barrier is considered to be the key reason for the failure of the active hydrogenase production in heterologous hosts. The second group of genes is another set of the hyp (‘p’ for pleiotrophic) genes which encode proteins involved in the insertion of Ni, Fe, CO and CN into the active site of hydrogenase enzymes (Chaudhuri, A., and A. I. Krasna. 1990. Gen. Microbiol. 136:1153-1160; Dernedde, J. et al. 1996. Eur. J. Biochem. 235:351-358; Jacobi, A. et al. 1992. Arch. Microbiol. 158:444-451; Maier, T. et al. 1996. Arch. Microbiol. 165:333-341; Wolf, I. et al. 1998. Arch. Microbiol. 170:451-459, each of which is incorporated herein by reference in its entirety). Mutations of these genes pleiotropically affect the synthesis and activity of all the hydrogenase isoenzymes. However, the functions of this set of genes can be complemented in trans by heterologous genes (Chaudhuri, A., and A. I. Krasna. 1990. supra).

Thus far, only few cases of studies have been reported in which functional hydrogenases were successfully expressed in heterologous hosts (Asada, Y. et al. 2000. Biochim. Biophys. Acta-Gene Struct. Expression 1490:269-278; Rousset, M. et al. 1998(b). Plasmid 39:114-122, each of which is incorporated herein by reference in its entirety; Boehm, R. et al. 1990. supra). The first case was the expression of the Fe-only hydrogenase from the strict anaerobe bacterium C. pasteurianum, enhancing the H₂ evolution of the expression host cyanobacterium Synechococcus sp. PCCC7942 (Asada, Y. et al. 2000. supra). The second case of heterologous expression involved a cloned [NiFe] hydrogenase and was achieved by interspecies transfer of the hyn genes from the Desulfovibrio gigas into Desulfovibrio fructosovorans MR400, although only low activity was observed (Rousset, M. et al. 1998(b). supra). The third case involved the expression of a functional NAD⁺-reducing [NiFe] hydrogenase from the gram-positive Rhodococcus opacus in gram-negative R. eutropha (Porthun, A. et al. 2002. Arch. Microbiol. 177:159-166, which is incorporated herein by reference in its entirety). A plasmid carrying the four subunit genes and an accessory gene of a bidirectional NAD⁺-reducing [NiFe] hydrogenase from R. opacus was transformed into an R. eutropha mutant impaired in H₂-oxidizing ability, restoring lithoautotrophic growth.

Despite these limited successes, none of the above-referenced expression hosts are conventional expression hosts. Non-conventional expression hosts are difficult to culture and genetically manipulate (Rousset, M. et al. 1998(a). Plasmid 39:114-122, which is incorporated herein by reference in its entirety). In addition, although subunits or domains of several hydrogenases have been heterologously expressed in E. coli, functional hydrogenases have not been produced in this conventional host (Atta, M. et al. 1998. Biochemistry 37:15974-15980; Deluca, G. et al. 1998. Biochemistry 37:2660-2665; Mura, G. M. et al. 1996. Microbiology 142:829-836; Verhagen, M. et al. 2001. Biochim. Biophys. Acta-Bioenerg. 1505:209-219; Voordouw, G. et al. 1987. Eur. J. Biochem. 162:31-36, each of which is incorporated herein by reference in its entirety; Asada, Y. et al. 2000; Rousset, M. et al. 1998(b). supra). Heterologous expression of active hydrogenases failed in most cases due to protein-associated maturation processes in the assembly of the active centers. Consequently, engineering hydrogenase with desirable electrochemical properties has been a nearly untapped area. Metabolic engineering can hold the answer to these production and expression problems by providing a way to eliminate bottle necks and to engineer the maturation pathway of heterologous hydrogenases (Bailey, J. E. 1991. Science 252:1668-1675; Chittibabu, G. et al. 2006. Process Biochem. 41:682-688; Kumar, N. et al. 2001. Biotechnol. Lett. 23:537-541; Li, Q. Z., and G. Y. Wang. 2005. Hydrogen and hydrogen bioelectrocatalyst production in synthetic E. coli strains, Industrial Microbiology and Biotechnology. Society for Industrial Microbiology, Chicago, Ill.; Stafford, D. E., and G. Stephanopoulos. 2001. Curr. Opin. Microbiol. 4:336-340, each of which is incorporated herein by reference in its entirety).

### Hydrogen Utilization and Candidate Hydrogenases for Expression in Heterologous Hosts

The proteobacterium Ralstonia eutropha H 16 (formerly Alcaligenes eutrophus) is one of the best studied facultative chemolithoautotrophs and well adapted to the changing chemical environment (Lenz, O., and B. Friedrich. 1998. Proc. Natl. Acad. Sci. USA 95:12474-12479, which is incorporated herein by reference in its entirety). It can grow on wide ranges of organic substrates and alternatively utilizes H₂ as the sole energy source (Friedrich, B., and E. Schwartz. 1993. supra).

R. eutropha possesses two energy-linked [NiFe] hydrogenases: a membrane-bound hydrogenase (MBH) and a soluble cytoplasmic hydrogenase (SH). The MBH is primarily involved in electron transport-coupled phosphorylation through coupling to the respiratory chain via a b-type cytochrome, whereas the SH is able to reduce NDA⁺ to generate reducing equivalents (Schink, B., and H. G. Schlegel. 1979. Biochim. Biophys. Acta 567:315-324; Schneider, K., and H. G. Schlegel. 1976. Biochim. Biophys. Acta 452:66-80, each of which is incorporated herein by reference in its entirety). The genes encoding the two hydrogenases are clustered in two separate operons (SH and MBH) together with accessory and regulatory genes involved in hydrogenase biosynthesis on megaplasmid pHG1, which has recently been completely sequenced (Schultz, M. G. et al. 2003. Science 302:624-627; Schwartz, E. et al. 1998. J. Bacteriol. 180:3197-3204, each of which is incorporated herein by reference in its entirety). The SH operon comprises the structural genes (hoxFUYH) of the heterotetrameric hydrogenase, two accessory genes (hoxW, hoxI) (Schwartz, E. et al. 2003. J. Mol. Biol. 332:369-383, which is incorporated herein by reference in its entirety). The MBH operon consists of the structural genes (hoxKGZ) and accessory genes (hoxMLOZRTV) (Bernhard, M. et al. 1997. Eur. J. Biochem. 248: 179-186, which is incorporated herein by reference in its entirety). The precise function of most of the conserved MBH accessory genes is not known. Unlike the ‘regular’ oxygen-sensitive hydrogenases from other organisms, the physiologically distinct [NiFe] hydrogenases of R. eutropha are fully active in the presence of molecular oxygen (Lenz, O., et al. 2002. J. Mol. Microbiol. Biotechnol. 4:255-262, which is incorporated herein by reference in its entirety). Recently, it was discovered that one of four cyanides is responsible for the insensitivity of SH towards oxygen.

A third hydrogenase, regulatory hydrogenase (RH), was identified in R. eutropha and classified as belonging to the subclass of H₂-sensing [NiFe] hydrogenases (Kleihues, L. et al. 2000. J. Bacteriol. 182:2716-2724; which is incorporated herein by reference in its entirety; Lenz, O., and B. Friedrich. 1998. supra). The RH is stable in presence of O₂, CO, and C₂H₂. However, its rate of hydrogen oxidation is one to two orders of magnitude lower than that of standard [NiFe] hydrogenase (Pierik, A. J. et al. 1998. FEBS Lett. 438:231-235, which is incorporated herein by reference in its entirety). The RH contains an active size like that in standard [NiFe] hydrogenase. Unlike the ‘standard’ hydrogenases, the RH has only two active states Niₐ—S and Niₐ—C*. Furthermore, the RH possesses only one binding sites for H₂ while normal [NiFe] hydrogenases have two such sites (Coremans, J. M. C. C. et al. 1992. Biochim. Biophys. Acta 1119:157-168, which is incorporated herein by reference in its entirety). The hoxB and hoxC genes encode the large and small subunit, respectively, of RH. The hyp genes (hypA1B1F1CDEX) are responsible for the maturation of RH in R. eutropha are located between the MBH genes and hoxA. To distinguish the hyp genes of R. eutropha from those of E. coli, hyp genes from R. eutropha are herewith designated as hyp_{RE} while those from E. coli are herewith designated as hyp_{EC}.

As herein described, MBH, SH and RH are produced in genetically engineered E. coli strains, and the catalytic properties are improved using protein engineering approaches.

Biohydrogen Production in E. coli

The model organism and facultative anaerobe, Escherichia coli, is a well-known microbial host for the production of diverse chemicals and proteins. E. coli is capable of three alternative modes of energy generation: aerobic respiration, anaerobic respiration, and fermentation. It utilizes two modes of hydrogen metabolism: (1) respiratory hydrogen oxidation (uptake) linked to quinine reduction, and (2) non-energy conserving hydrogen evolution during fermentative growth (Sawers, G. 1994. Antonie van Leeuwenhoek 66:57-88; Skibinski, D. A. G. et al. 2002. J. Bacteriol. 184:6642-6653, each of which is incorporated herein by reference in its entirety). Four hydrogenase isoenzymes have been identified in the E. coli genome. (Self, W. T. et al. 2004. J. Bacteriol. 186:580-587, which is incorporated herein by reference in its entirety). Two hydrogenases (hydrogenases 1 and 2) are involved in periplasmic hydrogen uptake, while the others (hydrogenases 3 and 4) are part of cytoplasmically oriented formate hydrogenase complexes that evolve hydrogen (Sawers, G. 1994. supra; Self, W. T. et al. 2004. supra; Sawers, R. G. 2005. Biochem. Soc. Transac. 33:42-46; Vignais, P. M. et al. 2001. FEMS Microbiol. Rev. 25:455-501, each of which is incorporated herein by reference in its entirety).

The uptake hydrogenases 1 and 2 are multi-subunit, membrane-bound, nickel-containing Fe/S proteins (Vignais, P. M. et al. 2001. supra). The function of hydrogenase 1 is thought to cycle hydrogen produced by hydrogenase 3 during fermentation (Sawers, G. 1994. supra). The hya operon encoding hydrogenase 1 comprises six open reading frames, hyaABCDEF (Menon, N. K. et al. 1990. J. Bacteriol. 172: 1969-1977, which is incorporated herein by reference in its entirety). Hydrogenase 1 is a transmembrane protein which is purified as a heterodimer of a 64 kDa large subunit and a 35-kDa small subunit (Sawers, R. G., and D. H. Boxer. 1986. Eur. J. Biochem. 156:265-276, which is incorporated herein by reference in its entirety). The hyaA gene encodes the 40.6 kDa Fe/S protein with a large N-terminal signal sequence. In its maturation process, the loss of the N-terminal signal results in the membrane-bound 35 kDa small subunit. The hyaB encodes the Ni/Fe-containing large subunit. The other genes encode the accessory proteins for the processing of these subunits into the active form. Hydrogenase 2 is involved in H₂-dependent fumarate reduction (Ballantine, S. P., and D. H. Boxer. 1986. Eur. J. Biochem. 156:277-284; Menon, N. K. et al. 1994. J. Bacteriol. 176:4416-4423; Sawers, R. G. et al. 1985. J. Bacteriol. 164: 1324-1331, each of which is incorporated herein by reference in its entirety). It is encoded by the hyb operon, containing 8 open reading frames, hybOABCEEFG (29, 44). The core catalytic dimer of hydrogenase 2 consists of the hybOC complex, in which hybC encodes 60 kDa large subunit and hybO encodes the 35 kDa small subunit (Menon, N. K. et al. 1994. supra; Sargent, F. et al. 1998. Eur. J. Biochem. 255:746-754, which is incorporated herein by reference in its entirety). The other genes encode its accessory proteins.

Under anaerobic conditions, E. coli cells carry out a mixed-acid fermentation and excrete formate (via the formate channel, FocA), acetate, succinate, lactate and ethanol when growing on glycolic carbon sources in absence of electron acceptors. Formate can be metabolized to H₂ and CO₂ by the membrane-associated formate dehydrogenase (FHL) complex.

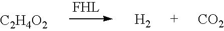

Thus, the FHL complex offsets the potentially deleterious effects of formate accumulation on fermentation by maintaining pH homeostasis (Boehm, R. et al. 1990. Mol. Microbiol. 4:231-244; Rossmann, R. et al. 1991. Mol. Microbiol. 5:2807-2814; Sauter, M. et al. 1992. Mol. Microbiol. 6:1523-1532, each of which is incorporated herein by reference in its entirety). The FHL 1 complex contains formate dehydrogenase H and the hydrogenase 3. This pathway is only active at low pH and high formate concentration and is thought to provide a detoxification/deacidification system countering the buildup of formate during fermentation. The seven subunits of the hydrogenase 3 are encoded by the hycABCDEFGHI operon (Sauter, M. et al. 1992. supra). The hycE and hycG genes encode the hydrogenase large subunit, containing the [NiFe] center, and the hydrogenase small subunit, respectively. The other gene encodes proteins related to the maturation of hydrogenase 3.

Analysis of the E. coli genome sequences has revealed the proton-translocating formate hydrogenase 4 (FHL-2), which is encoded by the hyfABCDEFGHIK operon (Andrews, S. C. et al. 1997. Microbiology 143:3633-3647; Bagramyan, K., and A. Trchounian. 2003. Biochem.-Moscow 68:1159-1170, each of which is incorporated herein by reference in its entirety). FHL-1 and FHL-2 appear to have different functions during fermentation. Under fermentation conditions at slightly acidic pH, the production of H₂ mostly results from the hydrogenase 3 that is part of FHL-1. On the other hand, at slightly alkaline pH, the H₂ production largely depends on the hydrogenase 4 that is part of FHL-2 (Bagramyan, K. et al. 2002. FEBS Lett. 516:172-178, which is incorporated herein by reference in its entirety; Bagramyan, K. et al. 2003. supra). However, expression of hydrogenase 4 is not significant in the wild-type strain (Self, W. T. et al. 2004. supra). Apparently, the function of the hydrogenase 4 needs to be further elucidated.

Up-regulation of the FHL system in E. coli has previously been reported by blocking the synthesis of the FHL complex repressor, HycA (Penfold, D. W. et al. 2003. Enzyme Microb. Technol. 33:185-189, which is incorporated herein by reference in its entirety). Using glucose as a substrate, this E. coli HD701 strain evolved hydrogen at twice the rate of its counter part wild-type strain. A combination of hycA inactivation with the overexpression of the FHL activator fhlA also reportedly produced a 2.8-fold increase in hydrogen activity compared to the wild-type (Yoshida, A. et al. 2005. Appl. and Environ. Microbiol. 71:6762-6768, which is incorporated herein by reference in its entirety). The engineering and regulation of these hydrogenases has the potential to lead to a strain of E. coli that is capable of producing large quantities of hydrogen (Chittibabu, G. et al. 2006. supra). However, large-scale manipulation of the E. coli genome using metabolic approaches has been not reported for biohydrogen and hydrogenase production. As herein described, we have metabolically engineered E. coli strains for the production of molecular hydrogen and the expression of active hydrogenases from R. eutropha and from C. acetobutylicum.

Biohydrogen Production Using Fe-Hydrogenase from Clostridium acetobutylicum

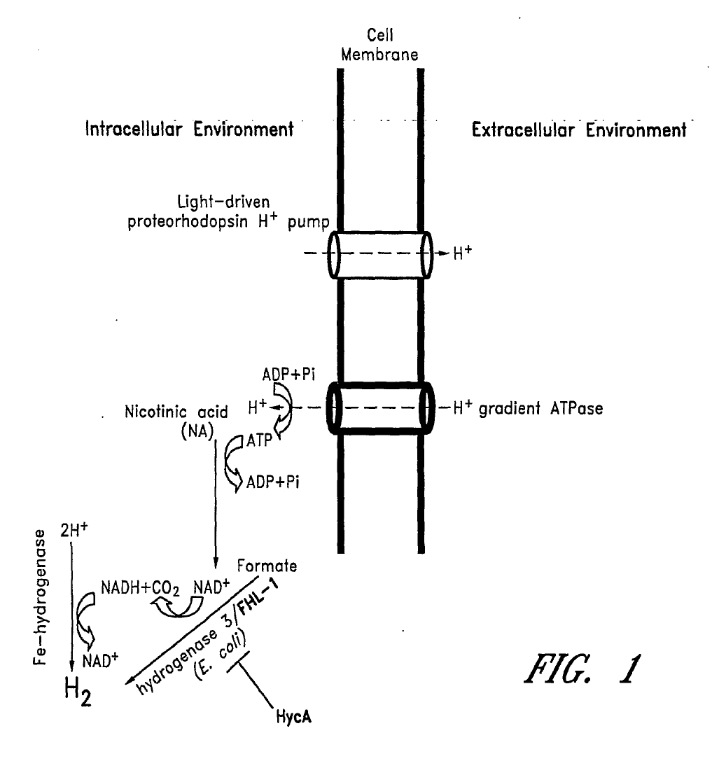

Fe-hydrogenase from C. acetobutylicum has been identified as one of the fastest hydrogen-evolving enzymes, which are found in many photosynthetic algae and anaerobic bacteria. The enzyme generates molecular hydrogen by oxidizing NADH to NAD⁺ (FIG. 1). It has been demonstrated that limiting iron (Fe) in cultures contributes to a decrease in hydrogenase concentration (Junelles, A. M. et al. 1988. Curr. Microbiol. 17:299-303; Peguin, S. and P. Soucaille. 1995. Appl. Environ. Microbiol. 61:403-405, each of which is incorporated herein by reference in its entirety). More recently, accessory genes hydEFG have been shown to be involved in biosynthesis of the active Fe-hydrogenase (HydA) from C. acetobutylicum (King, P. W. et al., 2006. J. Bacteriol. 188:2163-2172, which is incorporated herein by reference in its entirety). As herein described, Fe-hydrogenase from C. acetobutylicum can be expressed in E. coli to increase levels of hydrogen evolved by this common host.

Proteorhodopsin Proteins from Uncultured Marine Bacteria for ATP Generation

Proteorhodopsin (PR) is an integral membrane protein which binds retinal (vitamin A aldehyde) and functions in light-driven proton pumps in marine organisms (Béjà, O. et al. 2000. Science 289:1902-1906; Béjà et al. 2001. Nature 411:786-789, each of which is incorporated herein by reference in its entirety). It was first discovered on a large genome DNA fragment derived from uncultured marine γ-proteobacteria of the SAR86 group (Pernthaler, A. et al. 2002. Appl. Environ. Microbiol. 68:5728-5735, which is incorporated herein by reference in its entirety). In addition to proteobacteria, many types of marine plankton have also been found to harbor PR genes (Man, D. et al. 2003. EMBO J. 22:1725-1731); Sabehi G. et al. 2003. Environ. Microbiol. 5:842-849; Sabehi G. et al. 2004. Environ. Microbiol. 6:903-910; de La Torre, J. R. et al. 2003. PNAS 100:12830-12835; Venter, J. C. et al. 2004. Science 304:66-74, each of which is incorporated herein by reference in its entirety). E. coli cells harboring the proteorhodopsin gene acquire protons in the presence of retinal and light (Béjà, O. et al. 2000. supra). Recent analysis revealed that PR genes of BAC clones (e.g. MED66A03) are linked to a carotenoid biosynthesis gene cluster, which encodes proteins responsible for converting geranylgeranyl diphosphate to β-carotene (Sabehi G. et al. 2005. PLoS Biol. 3:1409-1417, which is incorporated herein by reference in its entirety). Furthermore, the gene coding for a homolog of the bacteriorhodopsin-related-protein-like homolog protein (Blh) from the archaeon Halobacterium sp. NRC-1 was found in the cluster clone. Blh has been shown to be involved in retinal biosynthesis (Peck, R. F. et al., 2001. J. Biol. Chem. 276:5739-5744). Expression of the blh gene in the β-carotene-producing E. coli cells induces the conversion of β-carotene to retinal (Sabehi, G. et al. 2005. supra). Thus, proteorhodopsin is able to couple the harvesting of light energy to the generation of a membrane potential. This generated membrane potential can then be used to synthesize ATP, which serves as energy currency for biological processes that include hydrogen evolution (FIG. 1).

Production and Function of Cofactors in E. coli for Hydrogen Evolution

The cofactor NADH plays a major role in cellular metabolism, and its availability can be a limiting factor in enzyme-catalyzed, cofactor-dependent production systems such as, for example, hydrogen evolution systems. It has been demonstrated that cofactor manipulations can be used as an additional tool for metabolic engineering (San, K. Y. et al. 2002. Metabolic Eng. 4:182-192, which is incorporated herein by reference in its entirety).

In E. coli, the total intracellular NADH/NAD⁺ pool is maintained by synthesizing NAD through two pathways: (1) the de novo pathway, and (2) the pyridine nucleotide salvage pathway. In the de novo pathway, NAD is synthesized from aspartate and dihydroxyacetone phosphate. The pyridine nucleotide salvage pathway produces NAD by recycling intracellular NAD metabolic products (e.g. nicotinamide mononucleotide (NMN)) and other preformed pyridine compounds from the environment (e.g. nicotinamide and nicotinic acid (NA)) in an ATP-dependent process (Susana, J. B., et al. 2002. Metabolic Eng. 4:238-247, which is incorporated herein by reference in its entirety). The gene pncB encodes the enzyme phosphoribosyl transferase (NAPTTase) in the salvage pathway. It has been shown that overexpression of the pncB gene from Salmonella typhimurium and addition of NA to minimal medium results in an increase in total intracellular NAD levels (Wubbolts et al., 1990. J. Biol. Chem. 265:17665-17672, which is incorporated herein by reference in its entirety). To balance production of NAD for enhancement of hydrogen production in E. coli, the bacterial cells can be engineered to overexpress, for example, phosphoribosyl transferase, Fe-hydrogenase from C. acetobutylicum, hydrogenase 3, or NAD⁺-dependent formate dehydrogenase (FDH) from Candida boidinii in order to increase NADH levels (Susana, J. B. et al., 2002. Metabolic Eng. 4:217-229, which is incorporated herein by reference in its entirety).

Generation of an E. coli Hydrogenase Null Strain for Engineering of Biocatalytic Hydrogenases and Optimization of Hydrogen Evolution

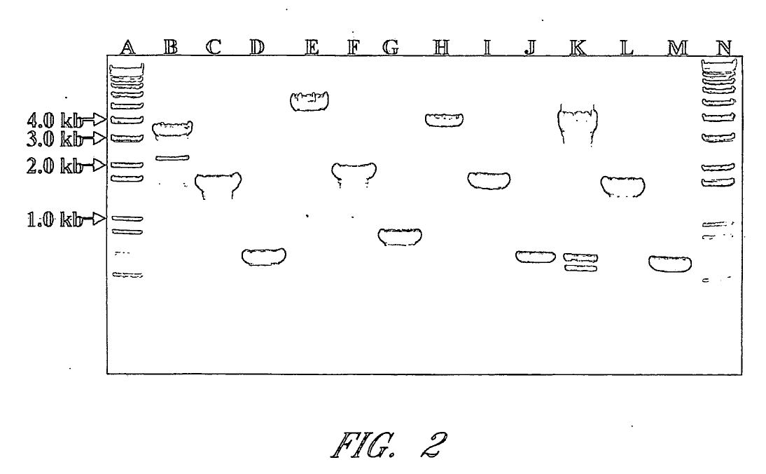

The E. coli genome harbors four hydrogen isoenzymes, hydrogenase 1, 2, 3, and 4 (Bagramyan, K. et al. 2002; Bagramyan, K., and A. Trchounian. 2003. supra). These indigenous hydrogenases can cause potential problems for the assay of any heterologously expressed hydrogenase enzyme. To avoid the potential problems of these indigenous hydrogenases, large and small subunits of hydrogenase 1, 2, 3, and 4 were deleted in the genome of the E. coli strain BW25113, generating the hydrogenase null strain (GW1234) using the red recombinase system (Datsenko, K. A., and B. L. Wanner. 2000. Proc. Natl. Acad. Sci. USA 97:6640-6645, which is incorporated herein by reference in its entirety). To verify the correct deletions of resulting strains, PCR primers were designed based on sequences flanking target genes to verify the completion of each gene knock-out (FIG. 2).

Enhancement of Hydrogen Production in Engineered E. coli Strains

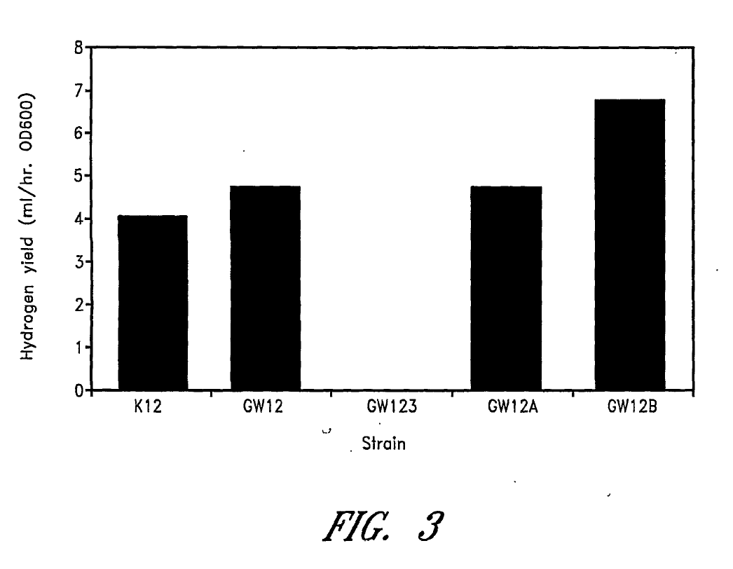

Hydrogenases 1 and 2 have been shown to be involved in periplasmic hydrogen uptake (Vignais, P. M. et al. 2001. supra). The strain GW12 (ΔhyaAB ΔhybABC), which does not express uptake hydrogenase 1 and 2, displayed increased hydrogen production by 17.1% under anaerobic fermentation conditions (Temp=37° C.; pH=7, dissolved oxygen=0.14%, CO₂=9.3%). See FIG. 3. In a further experiment, the gene hycA (SEQ ID NO: 61), which encodes repressor of hydrogenase 3 expression (Sauter, M. et al. 1992. supra), was deleted in GW12 for comparison of hydrogen evolution. The resulting strain GW12A displayed no significant increase in the hydrogen production rate in comparison with that of GW12. This is inconsistent with the report that the strain HD701 (ΔhycA) evolved several times more hydrogen than the wild-type parent strain MC4100 (Penfold, D. W. et al. 2003. supra). Currently, the hydrogen production rate of GW12A is being analyzed under different fermentation conditions.

To further increase the hydrogen production yield, genes encoding two subunits of hydrogenase 3 (hycEG) were overexpressed in strains GW12. The expression plasmid pHycEG was constructed by cloning the two genes of hycEG (SEQ ID NO: 62) into pTrc99A and transformed into E. coli strain GW12. The GW12 cells harboring pHycEG showed significant increase in hydrogen production rates compared with K12 and GW12 by 68.1% and 43.6%, respectively.

Construction and Expression of RH Biosynthetic Operons in Engineered E. coli for Production of Biocatalytic Hydrogenase

To express RH from R. eutropha in a genetically engineered E. coli strain, hoxBC genes were amplified from the megaplasmid pHG1, which was enriched from cultures of R. eutropha H16 grown in FN medium (Nies, D. et al. 1987. J. Bacteriol. 169:4865-4868, which is incorporated herein by reference in its entirety). Genes of HoxBC (hoxBC; SEQ ID NO: 72) were cloned into pBAD24. The expression plasmid pHoxBC was constructed by cloning the pBAD promoter and HoxBC into pBBR1MCS-3. At the same time, hyp_{RE} genes (SEQ ID NO: 66) were also amplified into two fragments from pHG1 via PCR. The two fragments were then assembled into one fragment using SOE-PCR (Horton, R. M. et al. 1990. Biotechniques 8:528-535, which is incorporated herein by reference in its entirety). The SOE products were cloned into pBAD33 to generate plasmid pRUhyp. The plasmid pHoxBC was transformed into the strain GW1234, the hydrogenase-null strain. In a separate transformation, pHoxBC and pRUhyp were co-transformed into strain GW1234. The empty vector pBAD24 was transformed into strain GW1234 as a control. Cells harboring these plasmids were cultured in LB broth at 37° C. and induced with 0.2% arabinose. Cells were then harvested by centrifugation and suspended with 50 mM Tris-HCl buffer (pH 7). The cell suspension was sonicated with Branson Sonifier 450 equipped with a double stepped microtip (3 mm, diameter). The resulting cell lysates were centrifuged at 14,000 rpm at 4° C. for 10 min. The supernatant was analyzed by uptake hydrogenase assay. The enzyme activity was detected by following the reduction of methyl viologen by hydrogen using a spectrophotometer (DeLacey, A. L. et al. 2003. J. Biol. Inorg. Chem. 8:129-134; Fernandez, V. M. et al. 1985. Biochim. Biophys. Acta 832:69-79, each of which is incorporated herein by reference in its entirety).

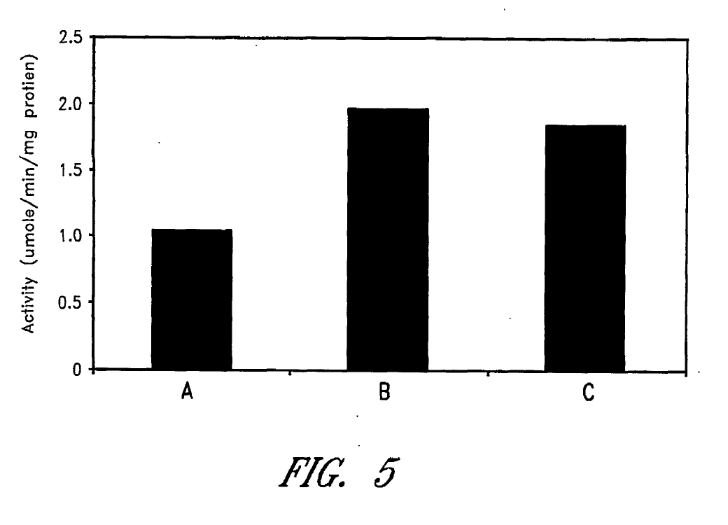

The GW1234 cells harboring pHoxBC displayed significant uptake hydrogen activity (FIG. 5). The cells containing pHoxBC and pRUhyp also showed similar uptake hydrogen activity, but slightly lower than that of cells harboring pHoxBC. The reduction of methyl viologen in the assay for cells harboring pBAD24 is a result indicative of activity from other redox proteins in the crude extract of the E. coli strain. Also, the low activity is ascribed to the nature of RH. Nevertheless, the results demonstrate that functional RH was expressed in the genetically engineered E. coli strain.

Construction and Expression of MBH and SH from R. eutropha for Production of Biocatalytic Hydrogenase

It has been demonstrated that a graphite electrode coated with membrane-bound hydrogenase (MBH) from R. eutropha can carry out rapid electrocatalytic oxidation of hydrogen. This oxidation reaction is unaffected by CO poisoning (conditions: pressure=0.9 bar, 9-fold excess of CO) and is only partially inhibited by oxygen (Vincent K. A. et al. 2005. PNAS. 102:16951-16954, which is incorporated herein by reference in its entirety). In order to construct and express an expression vector for MBH, the accessory genes for MBH (hoxMLOQRTV; SEQ ID NO: 75) and SH (hoxWI; SEQ ID NO: 76) were amplified from pHG1 into two DNA fragments using the following primers: hoxM-f, hoxVr, hoxW-f, hoxVI SOE-f, and hoxI-r (SEQ ID NO: 33 through 37). The two fragments were spliced together as a single operon via SOE-PCR (Horton, R. M. et al. 1990. supra). The SOE products were then cloned into pBAD33 to generate the plasmid pCISMBSH.

To express MBH in an engineered bacterial strain, the structural genes encoding MBH (hoxKGZ+pHG004, SEQ ID NO: 74) were amplified from pHG1 via PCR and cloned into pASK, generating the plasmid pHoxKGZ4. The engineered strain GW1234 was then co-transformed with pCISMBSH and pHoxKGZ4 to produce active MBH.

Likewise, to express SH in an engineered bacterial strain, the structural genes of soluble hydrogenase (SH) from R. eutropha (hoxFUYH; SEQ ID NO: 73) were also amplified from pHG1 via PCR and cloned into pASK, generating pHoxFUYH. The engineered strain GW1234 was then co-transformed with pCISMBSH and pHoxFUYH to produce active SH.

Construction and Expression of Fe-Hydrogenase (HydA) from Clostridium acetobutylicum for Hydrogen Evolution

Three genes (hydE, hydF and hydG) have recently been linked to the functional biosynthesis of Fe-hydrogenase (King, P. W. et al. 2006. J. Bacteriol. 188:2163-2172; Posewitz, M. C. et al. 2004. J. Biol. Chem. 279: 25711-25720, each of which is incorporated herein by reference in its entirety). To construct and express functional Fe-hydrogenase in engineered bacterial strains, the genes hydA, hydE, hydF and hydG (SEQ ID NO: 77 through 80) were isolated and amplified as four separate fragments from the genomic DNA of C. acetobutylicum using the following primers: CaHydA-f, CaHydA-r, CaHydE-f, CaHydE-r, CaHydF-f, CaHydF-r, CaHydG-f and CaHydG-r (SEQ ID NO: 45 through 52). The four fragments were then spliced together as a single operon via SOE-PCR (Horton, R. M. et al. 1990. supra) using the primers hydAE SOE-r, hydFG SOE-r, and hydAE/FG SOE-r (SEQ ID NO: 53 through 55). The SOE products were cloned into pBAD24 and pTrc99A, generating the plasmid pHYDCH24 and pHYDCH99, respectively. The bacterial strains GW12, GW12A and GW1234 transformed with either pHYDCH24 or pHYDCH99 can yield significant increases in hydrogen production.

Engineering “Mixotrophic” E. coli for Hydrogen Production

The gene cluster encoding a light-driven proton pump from the BAC (Bacterial artificial chromosome) clone MED66A03 (Sabehi, G. et al. 2005. supra) was subcloned using BAC Subcloning Kit (Gene Bridges) according to the manufacturer's manual, generating the plasmid pEIBYBP. To place the gene cluster under control of the synthetic promoter eS1 (Salaiman, D. K. Y and G. A. Somkuti. 1995. Appl. Microbiol. Biotechnol. 43:285-290, which is incorporated herein by reference in its entirety), the 63-bp eS1 promoter was spliced together with the minimal vector of the kit (Gene Bridges) before the subcloning procedure. Using the Quick and Easy Conditional knockout kit (Gene Bridges), the synthetic operon, which include the promoter eSI and the genes encoding the light-driven proton pump (crtE, crtI, crtB, crtY, blh and pr), was integrated into the genomes of engineered bacterial strains GW12A and GW1234, generating new strains GW12AP and GW1234P, respectively.

E. coli can maintain its total NADH/NAD⁺ intracellular pool by synthesizing NAD through the de novo pathway and the pyridine nucleotide salvage pathway. The first gene encoding phosphoribosyl transferase (NAPTTase) in the salvage pathway (pncB) is tightly controlled at the transcription level. E. coli bacteria harboring plasmid pSBN, which carries the pncB gene from Salmonella typhimurium (SEQ ID NO: 71) under control of its native promoter, has been shown to increase NAD⁺ production levels using ATP as an energy source (Berrios-Revera et al. 2002. Metabolic Eng. 4:238-247, which is incorporated herein by reference in its entirety). Plasmid pSBN is integrated into the genomes of strains GW12AP and GW1234P using the kit, generating new strains GW12APN and GW1234PN, respectively. Consequently, the resulting strains can use light as an energy source to produce ATP, which can be used to produce NAD⁺. Finally, the increased NAD⁺ can be used to produce H₂ via the E. coli hydrogenase 3 (FIG. 1).

Recombinant Evolution of Hydrogen Using Engineered E. coli

A reactor system can be set up to produce hydrogen from engineered hydrogen-evolving microorganisms. For example, strains GW12 and GW12A, can be cultured under standard fermentation conditions to produce hydrogen for use in, for example, fuel cells. In some embodiments, the microorganisms are cultured at from about 10° C. to about 45° C., more preferably from about 25° C. to about 45° C., most preferably from about 35° C. to about 45° C. The microorganisms can be cultured in standard media such as, for example, LB media. The media optionally includes appropriate antibiotics, based on the antibiotic resistance of the cultured strain. Typically, the pH of the media is from about 3 to about 9, more preferably from about 5 to about 8.5.

A hydrogen gas collection system can be included in the reactor system such that the hydrogen gas generated is collected and is optionally stored for use. Alternatively, the generated hydrogen gas can be directed to a point of use, such as, for example, to a hydrogen fuel powered device. In some embodiments, a hydrogen gas collection unit includes one or more hydrogen gas conduits for directing a flow of hydrogen gas produced in the reactor system to a storage container or directly to a point of use. In other embodiments, a hydrogen gas conduit is optionally connected to a source of a sweep gas, wherein the hydrogen gas is collected using the sweep gas. An exemplary sweep gas is nitrogen. For example, as hydrogen gas is initially produced, a sweep gas can be introduced into a hydrogen gas conduit, flowing in the direction of a storage container or point of hydrogen gas use. In further embodiments, a hydrogen collection system can include a container for collection of hydrogen from the reactor system. In still other embodiments, a collection system can further include a conduit for passage of hydrogen. The conduit and/or container can be in gas flow communication with a channel provided for outflow of hydrogen gas from the reaction chamber.

Embodiments of the reactor system include primary and secondary fermentation reactors. An organism is used to carry out the primary fermentation reaction. For example, a primary reaction can include the anaerobic breakdown of sugar, feedstocks or organic wastes into formate by yeast or bacteria, wherein the formate is used as a substrate in a secondary fermentation reaction. The term “primary fermentation reaction” is used to describe a process that results in a by-product. A secondary fermentation reaction takes place when an organism metabolizes a by-product of a primary fermentation reaction. The by-product is used in what is termed herein a “secondary fermentation reaction” indicating that the by-product can be metabolized in order to produce hydrogen gas. Alternatively, the E. coli strains can be engineered to utilize biomass, such as, for example, cellulose, hemicellulose and the like, to dramatically lower the production cost of hydrogen. Research is currently being conducted to investigate these options.

### Recombinantly-Produced Biocatalysts in Fuel Cells and Their Operation

As herein described, the fuel cell of the present subject matter is envisaged as a source of electrical energy which can replace conventional platinum electrode-based fuel cells.

Fuel cells are electrochemical devices that convert the energy of a fuel directly into electrochemical and thermal energy. Typically, a fuel cell consists of an anode and a cathode, which are electrically connected via an electrolyte. A fuel such as, for example, hydrogen, is fed to the anode where it is oxidized with the help of an electrocatalyst. At the cathode, the reduction of an oxidant such as oxygen (or air) takes place. The electrochemical reactions which occur at the electrodes produce a current and thereby electrical energy. Commonly, thermal energy is also produced which may be harnessed to provide additional electricity or for other purposes. Currently, the most common electrochemical reaction for use in a fuel cell is that between hydrogen and oxygen to produce water. Molecular hydrogen itself can be fed to the anode where it is oxidized, and the electrons produced are passed through an external circuit to the cathode where oxidant is reduced. Ion flow through an intermediate electrolyte maintains charge neutrality. Fuel cells can also be adapted to utilize the hydrogen from other hydrocarbon sources such as methanol or natural gas.

The fuel cells of the present subject matter utilize hydrogen as a fuel. The source of hydrogen can be hydrogen gas itself, or the hydrogen can be derived from an alternative source such as an alcohol, including methanol and ethanol, or from fossil fuels such as natural gas. Typically, hydrogen itself is used. In some embodiments, the hydrogen is derived from the hydrogen product evolved from the engineered E. coli strains of the present subject matter. In other embodiments, the hydrogen is in a crude form and thus can contain impurities. In still other embodiments, purified hydrogen can be used.

The fuel source can be a gas which includes hydrogen and which is provided to the anode. In some embodiments, the fuel is provided in liquid form. Generally, the fuel source also includes an inert gas, although substantially pure hydrogen can also be used. For example, a mixture of hydrogen with one or more gases such as nitrogen, helium, neon or argon can be used as the fuel source.

The fuel source can optionally comprise further components, such as, for example, alternative fuels or other additives. The additives which can be present are preferably those which do not react with the catalyst, which is coated on the positive electrode. If other entities are present which react with the catalyst, these are made to be present in as small an amount as possible. For example, carbon monoxide (CO), which can react with the catalysts used in the present subject matter, is preferably present in an amount of less than about 30% by volume, more preferably less than about 10% by volume, for example less than about 5% or less than about 1% by volume. Higher concentrations of CO can lead to lower hydrogen oxidation currents. However, the effect of CO is reversible, and the removal of CO from the fuel gas can lead to the restoration of the oxidation current.

Typically, hydrogen is present in the fuel source in an amount of at least about 2% by volume, preferably at least about 5% and more preferably at least about 10% by volume, for example about 25%, 50%, 75% or 90% by volume. Where an inert gas is used to form part of the fuel gas, the inert gas is typically present in an amount of at least about 10%, such as at least about 25%, 50% or 75% by volume, most preferably at least about 80% by volume.

Generally, the fuel source is supplied from an optionally pressurized container of the fuel source in gaseous or liquid form. The fuel source is supplied to the electrode via an inlet, which can optionally comprise a valve. An outlet is also provided which enables used or waste fuel source to leave the fuel cell.

The oxidant typically includes oxygen, although any other suitable oxidant can be used. The oxidant source typically provides the oxidant to the cathode in the form of a gas which includes the oxidant. In some embodiments, the oxidant can be provided in liquid form. Generally, the oxidant source also includes an inert gas, although the oxidant in its pure form can also be used. For example, a mixture of oxygen with one or more gases such as nitrogen, helium, neon or argon can be used. The oxidant source can optionally comprise further components, for example alternative oxidants or other additives. An example of a suitable oxidant source is air.

Typically, oxygen is present in the oxidant source in an amount of at least about 2% by volume, preferably at least about 5% and more preferably at least about 10% by volume.

Generally, the oxidant source is supplied from an optionally pressurized container of the oxidant source in gaseous or liquid form. The oxidant source is supplied to the electrode via an inlet, which optionally comprises a valve. An outlet is also provided which enables used or waste oxidant source to leave the fuel cell.

The anode can be made of any conducting material for example stainless steel, brass or carbon, which can be graphite. The surface of the anode can, at least in part, be coated with a different material which facilitates adsorption of the catalyst. The surface onto which the catalyst is adsorbed is of a material which does not cause the hydrogenase to denature. Suitable surface materials include graphite, such as, for example, a polished graphite surface or a material having a high surface area such as carbon cloth or carbon sponge. Materials with a rough surface and/or with a high surface area are generally preferred.

The cathode can be made of any suitable conducting material which will enable an oxidant to be reduced at its surface. For example; materials used to form the cathode in conventional fuel cells can be used. An electrocatalyst can, if desired, be present at the cathode. This electrocatalyst can, for example, be coated or adsorbed on the cathode itself, or it can be present in a solution surrounding the cathode. Suitable electrocatalysts include those used in conventional fuel cells such as platinum. Biological catalysts can also be used for this purpose.

The catalyst includes one, or a mixture of, hydrogenases. The catalyst can also include further additives, if desired. Suitable hydrogenases include those having a [Ni—Fe] and/or [Fe—Fe] active site, preferably a [Ni—Fe] active site. Hydrogenases having a [Ni—Fe] and/or [Fe—Fe] active site are found in many microorganisms and are reported to enzymatically catalyze the oxidation and/or reduction of hydrogen in those microorganisms. Examples of the microorganisms containing hydrogenases include methanogenic, acetogenic, nitrogen-fixing, photosynthetic, such as purple photosynthetic, and sulfate-reducing bacteria and those from purple photosynthetic bacteria are preferred. Examples of suitable hydrogenases include, but are not limited to, the hydrogenases from R. eutropha and Fe-hydrogenase from C. acetobutylicum.

The microorganism discussed above can generally be obtained commercially. The microorganism can be cultured to provide a sufficient quantity of enzyme for use in the fuel cell. This can be carried out, for example, by culturing the enzyme in a suitable medium in accordance with known techniques. Cells can then be harvested, isolated and purified by any known technique.

The catalyst containing a hydrogenase is adsorbed onto the anode. This ensures that the hydrogenase is in direct electronic contact with the anode. The term “direct electronic contact”, as used herein, means that the catalyst is able to exchange electrons directly with the electrode. In this manner, the fuel cell of the present subject matter can operate without the need for an independent electron mediator to transfer charge from the catalyst to the electrode. A further advantage of the adsorption of the catalyst onto the anode resides in the availability of the hydrogenase for reaction. Adsorption of the catalyst onto the electrode avoids a rate-limiting diffusion step through the solution to the electrode in order for a reaction to take place. Further, the hydrogenase can be present in either an active or inactive state. A low electrode potential, such as is found at the anode surface, encourages the existence of the active site. Thus, hydrogenase molecules which are adsorbed to the anode are generally activated as long as the conditions are favorable.

The anode can be immersed in a suitable medium. This medium can be a solution of the catalyst, or an alternative medium, such as water, which does not contain hydrogenase or contains only very low concentrations of hydrogenase. If hydrogenase is present in the medium, exchange can take place between the hydrogenase molecules adsorbed to the-anode and those in solution. To avoid the exchange of active molecules at the anode with inactive molecules in solution, the concentration of hydrogenase in the medium is minimized. The concentration of hydrogenase in the medium is preferably kept at a minimum, preferably below about 1 mM, more preferably below about 0.1 μM or 0.01 μM.

Typically, the catalyst layer is adsorbed to the surface of the electrode using an attachment means. The attachment means is typically a polycationic material. Examples of suitable attachment means include large polycationic materials such as, for example, polyamines including polymixin and neomycin. The catalyst can be attached to the electrode surface as a submonolayer, a monolayer or as multiple layers, for example 2, 3, 4 or more layers. Preferably, at least about 10% of the available surface of the anode is coated with catalyst. The “available surface” of the anode is the surface which is in contact with the fuel source. More preferably, at least about 25%, 50% or 75% and particularly preferably at least about 90% of the available surface of the anode is coated with catalyst.

Any suitable technique for preparing and coating the anode can be used. Where the surface of the anode is a polished graphite surface, this surface can be polished using a suitable polishing means, for example an aqueous alumina slurry, prior to coating with the catalyst. Coating can be carried out by, for example, directly applying a concentrated solution of catalyst, optionally mixed with an attachment means, to the electrode surface, such as, for example, by pipette. Alternatively, the catalyst, optionally together with the attachment means, can be made up into a dilute aqueous solution (for example, about 0.1 to about 1.0 μM solution of hydrogenase). The electrode is then inserted into the solution and left to stand. A potential can be applied to the electrode during this period if desired. The potential enables the degree of coating with the catalyst to be easily monitored. Typically, the potential will be increased and then subsequently decreased within a range of from approximately −0.5 to 0.2V vs. SHE and the potential cycled in this manner for up to about 10 minutes at a rate of about 0.01 V/s, typically for about 5 or 6 minutes.

The fuel cells of the present subject matter include an electrolyte suitable for conducting ions between the two electrodes. The electrolyte is preferably one which does not require the fuel cell to be operated under extreme conditions which would cause the hydrogenase to denature. Thus, electrolytes which rely on high temperature or extreme pH are avoided. Other than these requirements, any suitable electrolyte can be used for this purpose. For example, a proton exchange membrane such as Nafion™ can be used or any other suitable electrolyte which is known in the art.

The conditions under which the fuel cell is operated are important in terms of the amount of current that can be generated from the cell. In particular, the conditions are an important consideration in keeping the hydrogenase in its active state. The presence of oxidants is one condition which causes inactivation of the hydrogenase. Thus, the anode of the fuel cell having catalyst adsorbed thereon is physically separated from the oxidant.

The partial pressure of hydrogen supplied to the anode and the pH of the medium surrounding the anode also affect the active state of the hydrogenase. Preferably, the conditions are maintained such that the maximum amount of hydrogenase is maintained in the active state. For example, at least about 50%, preferably at least about 70%, 80%, 90% or 95% of the hydrogenase adsorbed to the anode is in the active state. This can be achieved by adjusting the conditions such that the potential at the anode is not above about 0.3V vs. SHE, preferably not above about 0.2V, 0V or −0.2V or −0.4V, all vs. SHE.

The pH of any medium which is in contact with the hydrogenase is typically maintained at approximately 7. However, the pH can generally be from approximately 6 to 8, typically from about 6.5 to 7.5. Variation within these limits can be used to increase the proportion of hydrogenase which is in the active state.

The partial pressure of hydrogen which is supplied to the anode can also be varied to ensure that the hydrogenase is active. An increased partial pressure can maintain the hydrogenase in its active form. Suitable hydrogen partial pressures for use in the cell are at least about 1×10⁴ Pa, preferably at least about 2×10⁴ Pa, such as at least about 5×10⁴, 1×10⁵ or 1×10⁶ Pa.

The fuel cell of the present subject matter is typically operated at a temperature of at least about 25° C., more preferably at least about 30° C. It is preferred that the fuel cell is operated at a temperature of from about 35° C. to about 65° C., such as from about 40° C. to about 50° C. A higher temperature increases the rate of reaction and leads to a higher oxidation current. However, temperatures which are above about 65° C. can lead to damage to the hydrogenase.

A fuel cell, as described above, can be operated under the conditions described above, to produce a current in an electrical circuit The fuel cell is operated by supplying hydrogen to the anode and supplying an oxidant to the cathode. The fuel cell of the invention is capable of producing current densities of at least about 0.5 mA, typically at least about 0.8 mA, 1 mA or 1.5 mA per cm² of surface area of the positive electrode. For example, the fuel cell of the invention can produce a current of at least about 2 mA, such as at least about 3 mA per cm² of surface area of the positive electrode.

Fuel cells are also described in U.S. patent application Ser. No. 10/562,198, published as U.S. Patent Application Publication No. 2006-0251959, which is incorporated herein by reference in its entirety.

Accordingly, an expression vector can be constructed to produce a biocatalyst for use in a fuel cell or fuel cell system. The fuel cell or fuel cell system uses hydrogen as a fuel source to generate electricity. The biocatalyst can be, for example, a hydrogenase enzyme. Embodiments of the present subject matter include an expression vector that contains a gene encoding a hydrogenase enzyme. In some embodiments, the hydrogenase enzyme is regulatory hydrogenase (RH) from R. eutropha. In other embodiments, the hydrogenase is any other hydrogenase that can be expressed in the engineered strains. For example, the hydrogenase can be membrane-bound hydrogenase (MBH) obtained from R. eutropha or soluble hydrogenase (SH) from R. eutropha.

The expression vector can be transformed into a conventional expression host for the production of hydrogenase for use in a fuel cell or fuel cell system. The host is typically a genetically engineered microorganism. In some embodiments, the host is E. coli strain BW25113 in which the gene for hydrogenase 1 is deleted from the genome. In other embodiments, the host is E. coli strain BW25113 in which the gene for hydrogenase 2 is deleted from the genome. Embodiments of the host also include E. coli strain BW25113 in which the gene for hydrogenase 3 is deleted from the genome. In still other embodiments, the host can be E. coli strain BW25113 in which the gene for hydrogenase 4 is deleted from the genome. In further embodiments, the host can be E. coli strain BW25113 in which at least two, three or all of the genes selected from the group of hydrogenase 1, 2, 3, and 4 are deleted from the genome. In preferred embodiments, the host is E. coli strain BW25113 in which the genes for hydrogenase 1, 2, 3 and 4 are deleted from the genome.

The expression host transformed with the expression vector can be cultured under standard culture conditions, and a hydrogenase product can be isolated and purified from the culture using standard protein purification techniques. The term “purified” does not require absolute purity; rather, it is intended as a relative definition. Isolated proteins have been conventionally purified to electrophoretic homogeneity by Coomassie staining, for example. Purification of hydrogenase to at least one order of magnitude, preferably two or three orders, and more preferably four or five orders of magnitude is expressly contemplated. In some embodiments of the invention, the term “purified” describes a hydrogenase of the subject matter which has been separated from other compounds including, but not limited to nucleic acids, lipids, carbohydrates and other proteins. A substantially pure hydrogenase typically comprises about 50%, preferably 60 to 90% weight/weight of a protein sample, more usually about 95%, and preferably is over about 99% pure. Protein purity or homogeneity is indicated by a number of means well known in the art, such as agarose or polyacrylamide gel electrophoresis of a sample, followed by visualizing a single polypeptide band upon staining the gel. For certain purposes higher resolution can be provided by using HPLC or other means well known in the art.

The hydrogenase product can be used, for example, in a fuel cell. The hydrogenase can be part of a catalyst that is in direct contact with an anode in a fuel cell. The fuel cell can be operated as described herein.

Furthermore, an expression vector can be constructed to produce hydrogen for use in a fuel cell or fuel cell system, wherein the fuel cell or fuel cell system uses hydrogen as a fuel source to generate electricity. Embodiments of the present subject matter include an expression vector that contains a gene encoding a hydrogenase enzyme. In some embodiments, the hydrogenase enzyme is hydrogenase 3 from E. coli. In other embodiments, the hydrogenase is Fe-hydrogenase from Clostridium acetobutylicum. In other embodiments, the hydrogenase is any other hydrogenase that can be expressed in the engineered strains. For example, the hydrogenase can be hydrogenase 4 obtained from E. coli. In still other embodiments, the expression vector contains genes that express at least one of the enzymes selected from the following group: hydrogenase 3 from E. coli, hydrogenase 4 from E. coli and Fe-hydrogenase from C. acetobutylicum.

A reactor system can be set up and used to produce hydrogen for use in a fuel cell or fuel cell system using a genetically engineered microorganism. In some embodiments, the microorganism is E. coli strain BW25113 in which the gene for hydrogenase 1 is deleted from the genome (strain “GW1”). In other embodiments, the microorganism is E. coli strain BW25113 in which the gene for hydrogenase 2 is deleted from the genome (strain “GW2”). Embodiments of the microorganism also include E. coli strain BW25113 in which the gene for hydrogenase 3 is deleted from the genome (strain “GW3”). In still other embodiments, the microorganism can be E. coli strain BW25113 in which the gene for hydrogenase 4 is deleted from the genome (strain “GW4”). In further embodiments, the microorganism can be E. coli strain BW25113 in which at least two, three or all of the genes selected from the group of hydrogenase 1, 2, 3, and 4 are deleted from the genome. In preferred embodiments, the microorganism is E. coli strain BW25113 in which the genes for hydrogenase 1 and 2 are deleted from the genome. In some embodiments, the microorganism. is optionally transformed with the expression vector containing genes that encode one or more of the following: hydrogenase 3 from E. coli, hydrogenase 4 from E. coli, Fe-hydrogenase from C. acetobutylicum. The reactor system is operated as described herein.

### Examples

The following examples are offered to illustrate, but not to limit, the claimed invention.

### Example 1

### Deletion of Hydrogenase Genes in E. coli

The genes for the large and small subunits of hydrogenase 1, 2, 3, and 4 (hyaAB, hybABC, hycEFG, hyfGHI, corresponding to respective SEQ ID NOs: 56, 57, 63, 64) as well as hydrogenase 3 repressor (hycA; SEQ ID NO: 61) were sequentially deleted from the genome of E. coli strain BW25113 to generate strains GW12, GW12A, GW123 and GW1234. The strains and their deletions are listed in Table 1. The resulting strain GW12 contains only hydrogen-evolving enzymes (hydrogenases 3 and 4), while the strain GW1234 is a hydrogenase-null strain. In addition, the strain GW12A further has the gene for hydrogenase 3 repressor (hycA; SEQ ID NO: 61) removed and is thus able to produce increased levels of hydrogen-evolving enzyme (hydrogenase 3).

Deletion of the genes was accomplished using the red recombinase system (Datsenko, K. A., and B. L. Wanner. 2000. supra). The genes targeted for deletion were amplified using primers (Table 2, SEQ ID NO: 1 through SEQ ID NO: 10) designed for the individual target genes. The PCR products were gel-purified using Qiagen Kit and assembled into gene-deletion cassette using SOE PCR (Horton, R. M. et al. 1990. supra). To verify the correct deletions of resulting strains, PCR primers were designed using the flanking sequences of the deleted genes and used to verify the completion of each gene knock-out. The PCR primers used for verification are listed in Table 3 (SEQ ID NO: 11 to SEQ ID NO: 20) based on sequences flanking target genes. The products from the PCR reactions were separated on 1% agarose gel. FIG. 2 illustrates the results of the PCR reactions and confirms that the gene knock-outs were accomplished (0.8% gel , lane A—1 kb ladder, lane B—hyaAB, lane C—ΔhyaAB::cat, lane D—ΔhyaAB, lane E—hybABC, lane F—ΔhybABC::cat, lane G—ΔhybABC, lane H—hycEFG, lane I—ΔhycEFG::cat, lane J—ΔhycEFG, lane K—hyfGHI, lane L—ΔhyfGHI::cat, lane M—ΔhyfGHI, lane N—1 kb ladder).

TABLE 1
Strains of bacteria used and/or generated

| Strain | Description | Ref. Source |
|---|---|---|
| E. coli K12 | | Wanner, B. L. |
| | | 1983 J. Mol. Biol. |
| | | 166: 283-308. |
| BW25113 | lac1^{q} rrnB_{T14} ΔlacZ_{WJ16} hsdR514 ΔaraBAD_{AH33} ΔrhaBAD_{LD78} | Datsenko, K. and |
| | | B. Wanner. 2000. |
| | | PNAS 97: 6640-6645. |
| GW1 | ΔhyaAB | this study |
| GW12 | ΔhyaAB ΔhybABC | this study |
| GW12A | ΔhyaAB ΔhybABC ΔhycA | this study |
| GW12B | Strain GW12 transformed with pHycEG | this study |
| GW123 | ΔhyaAB ΔhybABC ΔhycEFG | this study |
| GW1234 | ΔhyaAB ΔhybABC ΔhycEFG ΔhyfGHI | this study |
| GW0123 | ΔhyaAB ΔhybOABC ΔhycEFG ΔhyfGHI | this study |
| GW0123H | ΔhyaAB ΔhybOABC ΔhycEFG ΔhyfGHI Δhyp_{EC}::Δhyp_{RE} | this study |
| GW0123HE | ΔhyaAB ΔhybOABC ΔhycEFG ΔhyfGHI | this study |
| | Δhyp_{EC}::ΔP_{HPEC}hyp_{RE} | |
| GW0123HEP | ΔhyaAB ΔhybOABC ΔhycEFG ΔhyfGHI | this study |
| | Δhyp_{EC}::ΔP_{HPEC}hyp_{RE} ΔrpoH, Δlon, ΔompT | |
| GW12AP | ΔhyaAB ΔhybABC ΔhycA ΔhyaD:: ΔEIBYBP | this study |
| GW1234P | ΔhyaAB ΔhybABC ΔhycEFG ΔhyfGHI ΔhyaD:: ΔEIBYBP | this study |
| GW12APN | ΔhyaAB ΔhybABC ΔhycA ΔhyaD:: ΔEIBYBP ΔhyaF:: ΔpncB | this study |
| GW1234PN | ΔhyaAB ΔhybABC ΔhycEFG ΔhyfGHI ΔhyaD:: ΔEIBYBP | this study |
| | ΔhyaF:: ΔpncB | |

TABLE 2
Plasmids used and/or generated

| Plasmid | Description | Ref. Source |
|---|---|---|
| pBAD24 | broad-host expression plasmid; Amp^{R} | Guzman, L. M. et |
| | | al. 1995. J. |
| | | Bacterial. |
| | | 177: 4121-4130. |
| pBAD33 | broad-host expression plasmid; Cm^{R} | Guzman, L. M. et |
| | | al. 1995. supra. |
| pTcr99A | high-copy expression plasmid; Amp^{R} | Amann, E. et al. |
| | | 1988. Gene. |
| | | 69: 301-315. |
| pHycEG | pTrc99A derivative containing hycEG; Amp^{R} | this study |
| pHoxBC | pBBR1MCS-3 derivative containing hoxBC; Tc^{R} | this study |
| pRUhyp | pBAD33 derivative containing hyp_{RE}; Cm^{R} | this study |
| pHoxFUYH | pASK derivative containing HoxFUYH, Amp^{R} | this study |
| pHoxKGZ4 | pASK derivative containing HoxKGZ and pGH004, Amp^{R} | this study |
| pCISMBSH | pTrc99A derivative containing accessory genes of MBH and | this study |
| | SH from R. eutropha; Cm^{R} | |
| pHYDCH99 | pTrc99A derivative containing Fe-hydrogenase production | this study |
| | operon (hydAEFG); AmpR | |
| pHYDCH24 | pBAD24 derivative containing Fe-hydrogenase production | this study |
| | operon; Amp^{R} | |

TABLE 3
Sequences for genome deletions

| | | | SEQ ID |
|---|---|---|---|
| Primer | Sequence | Deletion | NO. |
| Hya-KO-F | atgaataacg aggaaacatt | hya deletion | 1 |
| | ttaccaggcc atgcggcgtc | | |
| | agggcgtgta ggctggagct | | |
| | gcttc | | |
| Hya-KO-R | agctcgctaccgtcgtcgccc | hya deletion | 2 |
| | agcacgtgtgttgaacaggcg | | |
| | aggcatatgaatatcctcctt | | |
| | ag | | |
| Hyb-KO-F | tgaacagacgtaattttatta | hyb deletion | 3 |
| | aagcagcctcctgcggggcat | | |
| | tgcgtgtaggctggagctgct | | |
| | tc | | |
| Hyb-KO-R | ttacagaaccttcactgaaac | hyb deletion | 4 |
| | cacttcgttgccgtcagcatc | | |
| | caccatatgaatatcctcctt | | |
| | ag | | |
| Hyc-KO-F | atgtctgaagaaaaattaggt | hyc deletion | 5 |
| | caacattatctcgccgcgctg | | |
| | aatgtgtaggctggagctgct | | |
| | tc | | |
| Hyc-KO-R | tcatcggatacgcgcctcttc | hyc deletion | 6 |
| | aacaacatgattcagatggct | | |
| | gaccatatgaatatcctcctt | | |
| | ag | | |
| Hyf-KO-F | aacgttaattcatcgtcaaat | hyf deletion | 7 |
| | cgtggcgaagcgattctcgcc | | |
| | gccgtgtaggctggagctgct | | |
| | tc | | |
| Hyf-KO-R | tcagtcatgtaacccccggag | hyf deletion | 8 |
| | tacgcgaaagagttgctgaac | | |
| | gatcatatgaatatcctcctt | | |
| | ag | | |
| hycA-KO-F | aaaaaatgcttaaagctggca | hycA deletion | 9 |
| | tctctgttaaacgggtaacct | | |
| | gacgtgtaggctggagctgct | | |
| | tc | | |
| hycA-KO-R | acactcatcgacacgcccatc | hycA deletion | 10 |
| | cccaaacaggcgtaacgcctg | | |
| | caa atatgaatatcctcctt | | |
| | ag | | |

TABLE 4
PCR primers

| | | | SEQ ID |
|---|---|---|---|
| Primer | Sequence | Purpose | NO. |
| Hya2-S-F | gcgacgtgcgccagtgcaga | hya deletion confirmation | 11 |
| Hya2-S-R | cggcacgataaacagctcgc | hya deletion confirmation | 12 |
| Hyb2-S-F | gcgctattggtttgctcggc | hyb deletion confirmation | 13 |
| Hyb2-S-R | agctgctatctcttcagtca | hyb deletion confirmation | 14 |
| Hyc2-S-F | ggtggactgctgctggcgc | hyc deletion confirmation | 15 |
| Hyc2-S-R | catgcagcatacttaacagc | hyc deletion confirmation | 16 |
| Hyf-S-F | tggctgcgcgggtgttaatggcg | hyf deletion confirmation | 17 |
| Hyf-S-R | gcttgctcctcttcgaccagtgc | hyf deletion confirmation | 18 |
| hycA-S-F | tgcggtgaat aatgtcgatg | hycA deletion confirmation | 19 |
| hycA-S-R | catcgacgcgggtgatggcg | hycA deletion confirmation | 20 |
| hycE-F | tctcgagctcatgtctgaagaaa | amplification of hycE | 21 |
| | aatta gg | | |
| hycE-R | tcatcagatggcctctttcaatg | amplification of hycE | 22 |
| | gcgattccttatttca | | |
| hycG-F | tgaaagaggccatctgatga | amplification of hycG | 23 |
| hycG-R | ctatgcatgccagttgactgaac | amplification of hycG | 24 |
| | accacct | | |
| RH-SOE-F1 | ccatggtaatgtctgacaagcag | amplification of hoxB | 25 |
| | gccactgttctt | | |
| RH-SOE-R1 | cagattctcggtcgccagcatca | amplification of hoxB | 26 |
| | ag | | |
| RH-SOE-F2 | cttgatgctg gcgaccgaga a | amplification of hoxC | 27 |
| | tctg | | |
| RH-SOE-R2 | tctagacggcaaccgatcaacgc | amplification of hoxC | 28 |
| | cggtctgcg | | |
| Hyp-SOE-F1 | gaattcaggaggatcgtccatgc | hyp SOE primer | 29 |
| | atgagctcagcctggcgggtgg | | |
| H-SOE-R1 | caatccggcttgcacgctggcgc | hyp SOE primer | 30 |
| | tc | | |
| Hyp-SOE-F2 | gagcgccagcgtgcaagccggat | hyp SOE primer | 31 |
| | tg | | |
| Hyp-SOE-R2 | aagcttagccgactcgctcaaca | hyp SOE primer | 32 |
| | aatgcgcgg | | |
| hoxW-f | atgaacgcgcccgctgag | primer for amplification of | 33 |
| | | SH accessory genes | |
| hoxI-r | gtcaagctt ctaaccccgtccc | primer for amplification of | 34 |
| | ctcc | SH accessory genes | |
| hoxM-f | atggtagttgcaatgggcattg | Primer for amplification of | 35 |
| | | MBH accessory genes | |
| hoxV-r | tcatgcatggacagtatctc | Primer for amplification of | 36 |
| | | MBH accessory genes | |
| hoxVI SOE-f | gagatactgtccatgcatgaatg | SOE primer for MBLI and | 37 |
| | aacgcgcccgctgag | SH accessory genes | |
| HoxK-f | agcccatggatggtcgaaacatt | MBH primer | 38 |
| | ttatgaag | | |
| HoxZa-r | agcaagctttcagcgaaaaaggg | MBH primer | 39 |
| | aaccgt | | |
| HoxF-f | agcccatggatggatagtcgtat | SH primer | 40 |
| | cacgac | | |
| NoxH-r | agcaagctttcagcgggcgcgtt | SH primer | 41 |
| | catc | | |
| Spmin1f | gaatgatataatggaaacatggt | eS1 and minimal vector | 42 |
| | atggatccgccgggagcggattt | SOE primer (Ref. 51) | |
| | gaacg | | |
| Spmin1r | ccgggagcagacaagcccg | eS1 and minimal vector | 43 |
| | | SOE primer (Ref. 19) | |
| Spmin2f | aattcggatccttttgcttttta | eS1 and minimal vector | 44 |
| | gctttaagaaatgatataatgga | SOE primer (Ref. 53) | |
| | aacatcg | | |
| CaHydA-f | agcccatggtgaaaacaataatc | Primer for amplifyication | 45 |
| | ttaaatggc | of hydA | |
| CaHydA-r | ttattcatgttttgaaacatttt | Primer for amplifyication | 46 |
| | tatc | of hydA | |
| CaHydE-f | atggataatataataaagttaat | Primer for amplification of | 47 |
| | taat | hydE | |
| CaHydE-r | ttaaccaatagattctttgtagc | Primer for amplification of | 48 |
| | | hydE | |
| CaHydF-f | atgaatgaacttaactcaacacc | Primer for amplification of | 49 |
| | | hydF | |
| CaHydF-r | ttagttcctactcgattgattaa | Primer for amplification of | 50 |
| | | hydE | |
| CaHydG-f | agcagtactatgtataatgttaa | Primer for amplification of | 51 |
| | atctaaagttg | hydG | |
| CaHydG-r | ttagaatctaaaatctctttgtc | Primer for amplification of | 52 |
| | c | hydG | |
| hydAE | attaattaactttattatattat | hydA and hydE SOE | 53 |
| SOE-r | ccatttattcatgttttgaaaca | primer | |
| | tttttatc | | |
| hydFG | caactttagatttaacattatac | hydF and hydG SOE | 54 |
| SOE-r | atttagttcctactcgattgatt | primer | |
| | aa | | |
| hydAE/FG | ggtgttgagttaagttcattcat | hydAB and hydEG SOE | 55 |
| SOE-r | ttaaccaatagattctttgtagc | primer | |

TABLE 5
Hydrogenase and hydrogenase-related genes

| | | SEQ ID |
|---|---|---|
| Gene | Purpose | NO. |
| hyaAB | encodes structural subunits of hydrogenase 1 | 56 |
| hybABC | encodes accessory proteins and large subunit of hydrogenase 2 | 57 |
| hyaD | encodes a maturation factor of hydrogenase 1 | 58 |
| hyaF | encodes a maturation factor of hydrogenase 1 | 59 |
| hybO | encodes small subunit of hydrogenase 2 | 60 |
| hycA | encodes repressor of hydrogenase 3 expression (HycA) | 61 |
| hycEG | encodes structural subunits for hydrogenase 3 | 62 |
| hycEFG | encodes structural subunits and an accessory gene for | 63 |
| | hydrogenase 3 | |
| hyfGHI | encodes structural subunits of hydrogenase 4 | 64 |
| hyp_{EC} | encodes maturation genes for hydrogenase in E. coli | 65 |
| hyp_{RE} | encodes maturation genes for metallocenter assembly of | 66 |
| | regulatory hydrogenase (RH) in R. eutropha | |
| rpoH (source: E. coli | encodes sigma subunit of RNA polymerase in E. coli | 67 |
| K12) | | |
| lon (source: E. coli | encodes the ATP-stimulated protease La in E. coli | 68 |
| K12) | | |
| ompT | encodes an outer membrane protease in E. coli | 69 |
| EIBYBP | encodes a gene cluster sequence for the light-driven proton | 70 |
| | pump from BAC clone MED66A03 | |
| pncB | encodes gene for phosphoribosyl transferase (NAPTTase) | 71 |
| | from S. typhimurium | |
| hoxBC | encodes structural subunits of regulatory hydrogenase (RH) | 72 |
| | from R. eutropha | |
| hoxFUYH | encodes structural subunits of soluble hydrogenase (SH) | 73 |
| | from R. eutropha | |
| hoxKGZ + pGH004 | encodes structural subunits of membrane-bound hydrogenase | 74 |
| | (MBH) from R. eutropha + small protein for MBH | |
| CISMBS | encodes accessory genes for membrane-bound hydrogenase | 75 |
| (hoxMLOQRTV) | (MBH) in R. eutropha | |
| CISSH (hoxWI) | encodes accessory genes for soluble hydrogenase (SH) in | 76 |
| | R. eutropha | |
| hydA | encodes active Fe-hydrogenase (HydA) in C. acetobutylicum | 77 |
| hydE | encodes accessory gene involved in the biosynthesis of | 78 |
| | active Fe-hydrogenase (HydA) in C. acetobutylicum | |
| hydF | encodes accessory gene involved in the biosynthesis of | 79 |
| | active Fe-hydrogenase (HydA) in C. acetobutylicum | |
| hydG | encodes accessory gene involved in the biosynthesis of | 80 |
| | active Fe-hydrogenase (HydA) in C. acetobutylicum | |
| hydAEFG | encodes an operon comprising hydA, hydE, hydF and hydG | 81 |
| | from C. acetobutylicum | |
| Phypeu | Encodes promoter of hyp genes from R. eutropha | 82 |
| PHYPEC | Encodes promoter of hyp genes from E. coli | 83 |

### Example 2

### Genetic Cloning of Regulatory Hydrogenase (RH)

For the expression of regulatory hydrogenase (RH) from R. eutropha, the genes hoxB and hoxC, which encode the large and small subunit of RH, respectively, was assembled into one expression unit, hoxBC (SEQ ID NO: 72), using the SOE PCR technique as described in Horton et al. (Horton, R. M. et al. 1990. supra). Briefly, megaplasmid pHG1 was enriched from cultures of R. eutropha H16 (ATCC17699) grown overnight in FN medium (Nies, D. et al. 1987. supra) and used as template for amplification of hoxB and hoxC. The two gene fragments were amplified using primers RH-SOE-F1/RH-SOE-R1 and RH-SOE-F2/RH-SOE-R2 (SEQ ID NOs: 25 through 28) before being spliced together as a single operon (hoxBC; SEQ ID NO: 72) using SOE PCR. Finally, pBAD promoter and hoxBC were cloned into pBBR1MCS-3, resulting in the expression plasmid pHoxBC.

In addition, using the megaplasmid pHG1 as a PCR template, DNA fragments containing the RH maturation genes hypA1B1F1 and hypCDEX were amplified using the primer pairs of Hyp-SOE-F1/Hyp-SOE-R1 and Hyp-SOE-F2/Hyp-SOE-R2 (Table 3, SEQ ID NO: 29 to SEQ ID NO: 32), respectively. The two fragments were assembled into hyp_{RE} (SEQ ID NO: 66) using SOE PCR as described in Horton et al. (Horton, R. M. et al. 1990. supra) and cloned into pBAD33, resulting in pRUhyp.

The plasmids were used to transform the engineered strain GW1234 (Example 1) using standard electroporation techniques, and the transformed strain was cultured in medium containing appropriate antibiotics (tetracycline, chloramphenicol).

### Example 3

### Genetic Cloning of Membrane Bound Hydrogenase (MBH)

For the expression of membrane bound hydrogenase (MBH) from R. eutropha, the structural genes encoding MBH were amplified from pHG1 via PCR. Briefly, megaplasmid pHG1 was enriched from cultures of R. eutropha H16 (ATCC17699) as described in Example 2 and used as template for amplification of hoxKGZ+pGH004, which encode the structural subunits for MBH and a small protein that complexes with MBH. The gene fragment was amplified using primers HoxK-f (SEQ ID NO: 38) and HoxZa-r (SEQ ID NO: 39). Finally, the structural genes encoding MBH and pGH004 (hoxKGZ+pGH004, SEQ ID NO: 74) were cloned into pASK, generating the plasmid pHoxKGZ4.

In addition, the accessory genes for MBH (hoxMLOQRTV; SEQ ID NO: 75) and soluble hydrogenase SH (hoxWI; SEQ ID NO: 76) were amplified from pHG1 in two separate DNA fragments using the following primers: hoxM-f, hoxVr, hoxW-f, hoxVI SOE-f, and hoxI-r (SEQ ID NO: 33 through 37). The two fragments were spliced together as a single operon via SOE-PCR (Horton, R. M. et al. 1990. supra). The SOE products were then cloned into pBAD33 to generate the plasmid pCISMBSH, which contains the gene cassette that encodes accessory genes for both MBH and SH.

To express MBH in an engineered bacterial strain, the engineered strain GW1234 (Example 1) is co-transformed with pCISMBSH and pHoxKGZ4 and cultured under standard fermentation conditions to produce active MBH.

### Example 4

### Genetic Cloning of Soluble Hydrogenase (SH)

For the expression of soluble hydrogenase (SH) from R. eutropha, the structural genes encoding SH were amplified from pHG1 via PCR. Briefly, megaplasmid pHG1 was enriched from cultures of R. eutropha H16 (ATCC17699) as described in Example 2 and used as template for amplification of hoxFUYH, which encode the structural subunits for SH. The gene fragment was amplified using primers Hoxf-f (SEQ ID NO: 40) and HoxH-r (SEQ ID NO: 41). Finally, the structural genes encoding SH (hoxFUYH, SEQ ID NO: 73) were cloned into pASK, generating the plasmid pHoxFUYH.

To express SH in an engineered bacterial strain, the engineered strain GW1234 (Example 1) is co-transformed with pCISMBSH (Example 3) and pHoxFUYH and cultured under standard fermentation conditions to produce active SH.

### Example 5

### Genetic Cloning of Hydrogenase 3

For the overexpression of hydrogenase 3, the genes hycE and hycG, which encode the large subunit and small subunit of hydrogenase 3, respectively, was assembled into one expression unit, hycEG (SEQ ID NO: 62), using SOE PCR (Horton, R. M. et al. 1990. supra.). Primers hycE-F/hycE-R and hycG-F/hycG-R (SEQ ID NOs: 21 through 24) were used to amplify the hycE and hycG genes using genomic DNA from E. coli strain BW25113 as template. The PCR products were then assembled into hycEG using SOE PCR and subsequently cloned into the plasmid pTrc99A, resulting in pHycEG. The plasmid was used to transform the engineered strain GW12 (Example 1) using standard electroporation techniques, producing strain GW12B. The transformed strain GW12B was cultured in medium containing ampicillin antibiotic.

The plasmid can also be used to transform engineered strains GW12A and GW1234 (Example 1) for culture under standard fermentation conditions in medium containing appropriate antibiotics (ampicillin) to produce hydrogen.

### Example 6

### Genetic Cloning of Fe-Hydrogenase

For expression of Fe-hydrogenase, the gene encoding Fe-hydrogenase (hydA; SEQ ID NO: 77) as well as the genes encoding accessory proteins for the biosynthesis of Fe-hydrogenase (hydE, hydF and hydG; SEQ ID NO: 78 through 80) were isolated and amplified as four separate fragments from the genomic DNA of C. acetobutylicum. The following primers were used for the amplification of the four gene fragments: CaHydA-f, CaHydA-r, CaHydE-f, CaHydE-r, CaHydF-f, CaHydF-r, CaHydG-f and CaHydG-r (SEQ ID NO: 45 through 52). The four fragments were then spliced together as a single operon via SOE-PCR (Horton, R. M. et al. 1990. supra) using the primers hydAE SOE-r, hydFG SOE-r, and hydAE/FG SOE-r (SEQ ID NO: 53 through 55). The SOE products were assembled into one operon (hydAEGF; SEQ ID NO: 81) cloned into pBAD24 and pTrc99A, generating the plasmids pHYDCH24 and pHYDCH99, respectively.

The plasmids are transformed into the engineered bacterial strains GW12, GW12A and GW1234 (Example 1) using standard electroporation techniques, and the transformed strains are cultured under standard fermentation conditions in medium contain appropriate antibiotics (ampicillin) for the production of hydrogen.

### Example 7

### Engineering of “Mixotrophic” Bacteria: Integration of Genes Encoding a Light-Driven Proton Pump

The gene cluster encoding a light-driven proton pump from the BAC (Bacterial artificial chromosome) clone MED66A03 (Sabehi, G. et al. 2005. supra) was subcloned using BAC Subcloning Kit (Gene Bridges) according to the manufacturer's manual, generating the plasmid pEIBYBP. To place the gene cluster (EIBYBP; SEQ ID NO: 70) under control of the synthetic promoter eS1 (Salaiman, D. K. Y and G. A. Somkuti. 1995. Appl. Microbiol. Biotechnol. 43:285-290, which is incorporated herein by reference in its entirety), the 63-bp eS1 promoter was spliced together with the minimal vector of the kit (Gene Bridges) before the subcloning procedure. The primers used to amplify the minimal vector and subclone the genes for the light-driven proton pump were Spmin1f (SEQ ID NO: 42), Spmin1r (SEQ ID NO: 43) and Spmin2f (SEQ ID NO: 44). Subsequent to the BAC subcloning procedure, the synthetic operon, which includes the promoter eS1 and the genes encoding the light-driven proton pump, was integrated into the genomes of engineered bacterial strains GW12A and GW1234 (Example 1) using the Quick and Easy Conditional knockout kit (Gene Bridges), generating new strains GW12AP and GW1234P, respectively. Upon transformation of strains GW12AP and GW1234P with plasmid pHycEG encoding hydrogenase 3 (Example 5) or with pHYDCH24 or pHYDCH99 encoding Fe-hydrogenase (Example 6), increased levels of H₂ are produced. To maintain the stability of the genes for hydrogenase 3 and/or Fe-hydrogenase in the engineered bacteria, strains GW12AP and GW1234P can be engineered such that the genes for hydrogenase 3 or Fe-hydrogenase are integrated into the host organism genome.

### Example 8

### Engineering of “Mixotrophic” Bacteria: Integration of Genes for Increased Production of NAD⁺ Cofactor

The cofactor NAD can be synthesized through the pyridine nucleotide salvage pathway using ATP as an energy source. The first gene encoding phosphoribosyl transferase (NAPTTase), the enzyme that catalyzes the NAD synthesis reaction in the pyridine nucleotide salvage pathway is pncB (SEQ ID NO:71). pncB has been cloned under control of its native promoter into a plasmid known as pSBN (Berrios-Revera et al. 2002. supra). Using the pSBN plasmid, pncB is integrated into the genomes of strains GW12AP and GW1234P (Example 7) using the Quick & Easy E. coli Gene Deletion Kit (Gene Bridges), generating new strains GW12APN and GW1234PN, respectively. The resulting strains use light as an energy source to produce ATP, which is subsequently used to synthesize NAD⁺. Upon transformation of strains GW12APN and GW1234PN with plasmid pHycEG encoding hydrogenase 3 (Example 5) or with pHYDCH24 or pHYDCH99 encoding Fe-hydrogenase (Example 6), increased levels of H₂ are produced. The production of hydrogen is enhanced in these transformed strains relative to the level produced by wild-type strains due to the engineered strains' ability to generate NAD⁺ using light energy. To maintain the stability of the genes for hydrogenase 3 and/or Fe-hydrogenase in the engineered bacteria, strains GW12APN and GW1234PN can be engineered such that the genes for hydrogenase 3 or Fe-hydrogenase are integrated into the host organism genome. See FIG. 1 for the biochemical pathway for hydrogen evolution.

### Example 9

### Measurement of Hydrogen Evolution

Hydrogen evolution measurements were performed on recombinant and engineered strains (Examples 1-3) by anaerobic batch fermentations using a 4 port assembly Bellco 500-ml spinner flask, filled with 250 mls of LB media only or LB media with antibiotics and grown at 37° C. in a circulating water bath. Fermentations were seeded by first making 1.8 ml aliquots consisting of the appropriate strain grown in LB or LB with appropriate antibiotics to an OD600 of 1.0 at 37° C. and stored at −80° C. with 16% glycerol. To start fermentation, an aliquot was individually thawed and injected into a spinner flask. Each spinner flask was equipped with a hydrogen sensor constructed from a modified OX 700 Clark-type oxygen probe and modified YSI 5300 Biological Oxygen Monitor as described previously (Coremans, J. M. C. C. et al. 1992. supra; Wang, R. et al. 1971. Plant Physiol. 48:108-110, which is incorporated herein by reference in its entirety).

Hydrogen evolution was also measured using gas chromatography by injecting a 200 μl volume of headspace gas into a G1540N/6890N network GC system (Agilent). The pH was monitored by using a Benson pH probe fitted in the spinner flask. Both pH and dissolved hydrogen measurements were recorded using LabVIEW software. Cell density was recorded by taking a 2-ml sample from the spinner flask and recording the OD at 600 nm.

### Example 10

### RH Activity Assay

Recombinant and engineered strains were grown in LB with appropriate antibiotic at 37° C. to an OD600 of 0.6 and induced with 0.2% arabinose for 5 hours at room temperature to induce expression of RH. For each strain, cells were harvested by centrifugation and suspended with 50 mM Tris-HCl buffer (pH 8). The cell suspension was sonicated using a Branson Sonifier 450 equipped with a double stepped microtip 3 mm in diameter. The resulting cell lysate was centrifuged at 14,000 rpm for 10 min at 4° C. The resulting supernatant was used for measurement of hydrogenase activity.

RH activity was detected spectrophotometrically by following the reduction of methyl viologen by hydrogenase according to the method described by Fernandez et al. (Fernandez, V. M. et al. 1985. supra). The total protein concentration was determined using the Bradford assay from BioRad (Hercules, Calif.) with BSA as the standard.

### Example 11

### MBH Activity Assay

Supernatant extracts from recombinant and engineered strains are prepared as described in Example 10 for the measurement of hydrogenase activity. MBH activity is detected spectrophotometrically by following the reduction of methyl viologen by hydrogenase according to the method described by Fernandez et al. (Fernandez, V. M. et al. 1985. supra). The total protein concentration is determined using the Bradford assay from BioRad (Hercules, Calif.) with BSA as the standard.

### Example 12

### SH Activity Assay

Supernatant extracts from recombinant and engineered strains are prepared as described in Example 10 for the measurement of hydrogenase activity. SH activity is determined by photometric recording of H₂-dependent NAH reduction (Schneider, K. et al. 1979, Biochim. Biophys. Acta 578:445-461, which is incorporated herein by reference in its entirety). The total protein concentration is determined using the Bradford assay from BioRad (Hercules, Calif.) with BSA as the standard.

### Example 13

### Hydrogen Production is Enhanced by Deletion of Uptake Hydrogenase Genes in the Engineered Strains

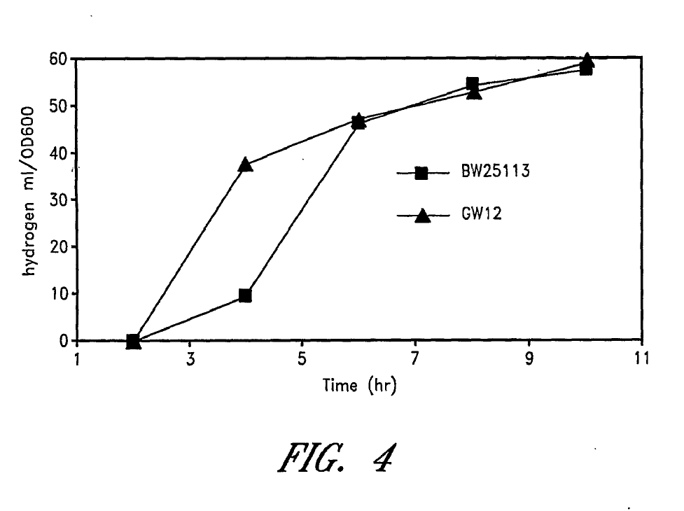

Hydrogen yields from batch fermentations were measured as described in Example 9 and plotted against each other to compare yields between each engineered strain. (See FIG. 3). Hydrogenase 1 and 2 have been shown to be involved in periplasmic hydrogen uptake (Sawers, R. G. 2005. supra); therefore, elimination of these two hydrogenases is predicted to increase hydrogen production. The strain GW12 (ΔhyaAB ΔhybABC) (Example 1), which does not express uptake hydrogenase 1 and 2, displayed a higher level of hydrogen production compared to wild-type E. coli strain BW25113 during the early stages of fermentation (FIG. 4).

The gene hycA (SEQ ID NO: 61) encodes a repressor of hydrogenase 3 expression (Yoshida, A. et al. 2005. supra). It has been reported that the E. coli strain HD701, which is deficient for the hycA gene and the genes for uptake hydrogenase 1 and 2 (hya and hyb, respectively), evolved several times more hydrogen than its parental strain MC4100 at glucose concentrations ranging from 3 to 200 mM (Penfold, D. W. et al. 2003. supra). Therefore, deletion of the hycA gene encoding this repressor in conjunction with eliminating uptake hydrogenase enzymes is predicted to significantly increase hydrogen production. However, the strain GW12A (ΔhyaAB ΔhybABC ΔhycA) (Example 1), which does not contain the uptake hydrogenase and hycA genes, displayed no significant increase in the hydrogen production rate in comparison with that of GW12. This is inconsistent with the report on the strain HD701(ΔhycA) (Penfold, D. W. et al. 2003. supra). Detailed characterization of GW12A is currently in progress. Further information and molecular data including, but not limited to, expression and translation levels of the genes hycE and hycG in GW12A and GW12 is being gathered to better understand the hydrogen evolution behavior of the strains.

FIG. 3 illustrates enhanced hydrogen production in engineered E. coli strains (E. coli BW25113—wild type, GW12—derivative of BW2513 (ΔhyaAB ΔhybABC), GW123—derivative of GW12 (ΔhyaAB ΔhybABC ΔhycEFG), GW12A—derivative of GW12 (ΔhyaAB ΔhybABC ΔhycA), and GW12B ΔhyaAB ΔhybABC containing plasmid pHycEG). E. coli strains were grown in standard nutrient broth supplemented with 100 mM glucose in shake flasks. Hydrogen production was detected using HP 6890 series GC system. Hydrogen yields were calculated as the average of two replicates.

### Example 14

### Hydrogen Production is Enhanced by Transformation of Engineered Strains with the Hydrogenase 3 Gene

To further increase the hydrogen production yield, the genes encoding two subunits of hydrogenase 3 (hycEG; SEQ ID NO: 62), were overexpressed in GW12 by transforming the plasmid pHycEG (Example 5) into GW12 (Example 1). The GW12 cells harboring pHycEG, referred to as strain GW12B (Example 5), showed significant increase in hydrogen production rates compared with wild-type and GW12 (by 68.1% and 43.6%, respectively).

Currently, conditions are being explored for the optimization of fermentation conditions for the evolution of hydrogen production in strains GW12, GW12A and GW12B. For example, limiting other byproducts from pyruvate in anaerobic conditions can be used to increase the overall production of biohydrogen in the engineered E. coli strains.

### Example 15

### RH Expression in E. coli

The E. coli hydrogenase-null strain GW1234 (Example 1) was transformed with the plasmids pHoxBC alone or with pRUhyp (Example 2). The uptake hydrogenase activity was determined spectrophotometrically as described in Example 10. The GW1234 cells harboring pHoxBC displayed significant hydrogen uptake activity, illustrating that RH is capable of being expressed in an active form (FIG. 5). Maturation genes have been reported to affect the expression of hydrogenase in heterologous hosts (Casalto, L., and M. Rousset. 2001. supra). The cells containing pHoxBC and pRUhyp showed similar uptake hydrogen activity, but slightly lower than that of cells harboring pHoxBC. The results indicate that the hyp_{RE} genes of R. eutropha are not necessary for the maturation of RH in E. coli, due to the native hyp_{EC} genes (SEQ ID NO: 65) still found in strain GW1234. The reduction of methyl viologen in the assay for control cells harboring pBAD24 indicates that other redox proteins are present in the crude extract of strain GW1234. Furthermore, the low activity was ascribed to the nature of RH. Nevertheless, functional RH was expressed in the genetically engineered E. coli strain.

Purified hydrogenases can provide more conclusive information on the results of the expression. Hydrogenase-null strain GW1234 is used to express strep-tagged RH hydrogenase and other microbial hydrogenases. Strep-tagged RH is purified using standard methods and materials such as, for example, Strep-tactin Superflow (Qiagen) or Gravity flow Strep-Tactin Superflow column (IBA GmbH). Other standard tags known in the art can also be used to purify RH.

FIG. 5 illustrates expression of RH in the genetically engineered E. coli strain (A, GW1234 cells containing pBAD24; B, GW1234 containing pHoxBC; C, GW1234 containing pHoxBC and pRUhyp). The values of enzyme activity were the average of two independent assays.

### Example 16

### Purification of RH from E. coli

PCR amplification of the hoxB and hoxC genes is conducted as described in Example 2. The resulting PCR products are then cloned into a standard plasmid containing a strep tag, such as, for example, pASK-IBA3 (IBA GmbH).

GW1234 cells are then transformed with the strep-tagged plasmid containing the hoxBC (SEQ ID NO: 72) sequence using standard transformation techniques. In some embodiments, the GW1234 cells are co-transformed with both the strep-tagged hoxBC plasmid and pRUhyp (Example 2). The transformed GW1234 cells are then cultured according to standard fermentation techniques with appropriate antibiotics.

At an appropriate cell density, the cell culture is harvested and lysed according to standard protocols. The strep-tagged RH protein is purified by standard column chromatography using an appropriate column material, such as, for example, Strep-Tactin Superflow resin.

### Example 17

### Purification of MBH from E. coli

PCR amplification of the hoxKGZ+pGH004 genes is conducted as described in Example 3. The resulting PCR product is then cloned into a standard plasmid containing a strep tag, such as, for example, pASK-IBA3 (IBA GmbH).

GW1234 cells are then transformed with the strep-tagged plasmid containing the hoxKGZ+pGH004 (SEQ ID NO: 74) sequence using standard transformation techniques. In some embodiments, the GW1234 cells are co-transformed with both the strep-tagged hoxKGZ+pGH004 plasmid and pCISMBSH (Example 3). The transformed GW1234 cells are then cultured according to standard fermentation techniques with appropriate antibiotics.

At an appropriate cell density, the cell culture is harvested and lysed according to standard protocols. The strep-tagged MBH protein is purified by standard column chromatography using an appropriate column material, such as, for example, Strep-Tactin Superflow resin.

### Example 18

### Purification of SH from E. coli

PCR amplification of the hoxFUYH gene is conducted as described in Example 4. The resulting PCR product is then cloned into a standard plasmid containing a strep tag, such as, for example, pASK-IBA3 (IBA GmbH).

GW1234 cells are then transformed with the strep-tagged plasmid containing the hoxFUYH (SEQ ID NO: 73) sequence using standard transformation techniques. In some embodiments, the GW1234 cells are co-transformed with both the strep-tagged hoxFUYH plasmid and pCISMBSH (Example 3). The transformed GW1234 cells are then cultured according to standard fermentation techniques with appropriate antibiotics.

At an appropriate cell density, the cell culture is harvested and lysed according to standard protocols. The strep-tagged SH protein is purified by standard column chromatography using an appropriate column material, such as, for example, Strep-Tactin Superflow resin.

### Example 19

### Use of Purified RH in a Fuel Cell

The purified RH (Example 16) is coated on one or more anodes in a fuel cell. The fuel cell is assembled as is known in the art. A hydrogen fuel source is fed to the fuel cell, and the fuel cell is operated at ambient temperature and neutral pH to produce a current in an electrical circuit.

### Example 20

### Use of Purified MBH in a Fuel Cell

The purified MBH (Example 17) is coated on one or more anodes in a fuel cell. The fuel cell is assembled as is known in the art. A hydrogen fuel source is fed to the fuel cell, and the fuel cell is operated at ambient temperature and neutral pH to produce a current in an electrical circuit.

### Example 21

### Use of Purified SH in a Fuel Cell

The purified SH (Example 18) is coated on one or more anodes in a fuel cell. The fuel cell is assembled as is known in the art. A hydrogen fuel source is fed to the fuel cell, and the fuel cell is operated at ambient temperature and neutral pH to produce a current in an electrical circuit.

### Example 22

### Bioproduction of Hydrogen

In a reactor system for bioproduction of hydrogen, an engineered strain containing the gene for hydrogenase 3 or Fe-hydrogenase (Examples 5, 6, 7, 8, and 14) is cultured under standard fermentation conditions and in standard fermentation media that includes appropriate antibiotics. A hydrogen gas collection system is included in the reactor system to collect and optionally store hydrogen for use. Alternatively, the generated hydrogen gas is directed to a point of use.

For example, the reactor system can include primary and secondary fermentation reactors. An organism is used to carry out the primary fermentation reaction, such as the anaerobic breakdown of complex sugar, feedstocks or organic wastes into simple sugars (e.g. glucose, fructose, sucrose, maltose) or formate by yeast or bacteria. The formate or simple sugar is used as a substrate in a secondary fermentation reaction to produce hydrogen gas. Alternatively, the E. coli strains can be engineered to utilize biomass, such as, for example, cellulose, hemicellulose and the like, to dramatically lower the production cost of hydrogen.

### Example 23

### Bioproduction of Hydrogen through Simultaneous Expression of Hydrogenase 3 and Fe-Hydrogenase in Engineered Bacteria

PCR amplification of the hycE and hycG genes and assembly into and assembly into hycEG is conducted as described in Example 5. The resulting gene sequence is cloned into a standard plasmid containing an antibiotic resistant selection marker.

PCR amplification of the hydA, hydE, hydF and hydG genes and assembly into hydAEFG is conducted as described in Example 6. The resulting gene sequence is cloned into a standard plasmid containing an antibiotic resistant selection marker that is different from that used for the plasmid containing hycEG.

Using standard electroporation techniques, the plasmids containing hycEG and hydAEFG are co-transformed into an engineered bacterial strains selected from the following group: GW12, GW12A and GW1234 (Example 1). In a reactor system for the bioproduction of hydrogen as described in Example 22, the transformed strain is cultured under standard fermentation conditions and in medium containing the appropriate different antibiotics.

### Example 24

### Bioproduction of Hydrogen through Simultaneous Expression of Hydrogenase 3 and Fe-Hydrogenase in Mixotrophic Engineered Bacteria

PCR amplification of the hycE and hycG genes and assembly into and assembly into hycEG is conducted as described in Example 5. The resulting gene sequence is cloned into a standard plasmid containing an antibiotic resistant selection marker.

PCR amplification of the hydA, hydE, hydF and hydG genes and assembly into hydAEFG is conducted as described in Example 6. The resulting gene sequence is cloned into a standard plasmid containing an antibiotic resistant selection marker that is different from that used for the plasmid containing hycEG.

Using standard electroporation techniques, the plasmids containing hycEG and hydAEFG are co-transformed into an engineered mixotrophic bacterial strain selected from the following group: GW12AP (Example 7), GW1234P (Example 7), GW12APN (Example 8) and GW1234PN (Example 8). In a reactor system for the bioproduction of hydrogen as described in Example 22, the transformed strain is cultured under standard fermentation conditions in medium containing the appropriate different antibiotics.

### Example 25

### Engineering Bacteria Strains to Partition Expression of Regulatory Hydrogenase from the Host Regulatory System

In the heterologous host, the native hyp promoter is controlled by the host regulatory system. To determine whether the expression of active regulatory hydrogenase (RH) can be carried out independently of the regulatory system of the E. coli heterologous host, the hyp_{EC} genes in E. coli (SEQ ID NO: 65) were replaced by the hyp_{RE} genes from R. eutropha (SEQ ID NO: 66) under control of either the E. coli hyp promoter (SEQ ID NO: 83) or the R. eutropha hyp promoter (SEQ ID NO: 82). The new strains were labeled GW0123H (hyp_{RE} under control of the E. coli hyp promoter) and GW0123HE (hyp_{RE} under control of the R. eutropha hyp promoter).

The newly-engineered E. coli strains are cultured under standard fermentation conditions, and activity of RH is measured from the separate strains as described in Example 10. Strain GW0123HE is found to produce increased levels of active RH compared to the strain GW0123H.

### Example 26

### Engineering Bacteria Strains to Improve Expression of Regulatory Hydrogenase by Deletion of Protease Genes

To determine whether the expression of active regulatory hydrogenase (RH) in strain GW123HE (Example 25) can be improved by deletion of host protease genes that can degrade the expressed product, the genes for rpoH (SEQ ID NO: 67), lon (SEQ ID NO: 68) and ompT (SEQ ID NO: 69) were deleted from the host genome to produce the new strain GW0123HEP.

The newly-engineered E. coli strain is cultured under standard fermentation conditions, and activity of RH is measured as described in Example 10. Strain GW0123HEP is found to produce increased levels of active RH compared to strains GW0123HE and GW0123H (Example 25).

It will be apparent to one skilled in the art that varying substitutions and modifications can be made to the invention disclosed herein without departing from the scope and spirit of the invention. It is recognized that various modifications are possible within the scope of the invention disclosed. Thus, it is understood that although the present invention has been specifically disclosed by preferred embodiments and optional features, modification and variation of the concepts herein disclosed can be resorted to by those skilled in the art, and that such modifications and variations are considered to be within the scope of this invention as defined by the disclosure.

Unless otherwise indicated, all numbers expressing quantities of ingredients, properties such as experimental conditions, and so forth used in the specification are to be understood as being modified in all instances by the term “about.” Accordingly, unless indicated to the contrary, the numerical parameters set forth in the specification are approximations that can vary depending upon the desired properties sought to be determined by the present invention.

1. An expression vector comprising one or more nucleic acid sequences associated with biosynthesis of a hydrogenase enzyme, wherein expression of the vector within a predetermined host results in altered hydrogenase activity that is different from native hydrogenase activity within the host.

2. The expression vector of claim 1, comprising one or more nucleic acid sequences encoding a hydrogenase enzyme or a fragment thereof.

3. The expression vector of claim 1, wherein the altered hydrogenase activity comprises elevated total hydrogenase activity within the host.

4. The expression vector of claim 1, wherein the altered hydrogenase activity comprises a hydrogenase activity with at least one distinct property as compared with the native hydrogenase activity within the host, and wherein the distinct property is selected from: increased enzyme yield, increased specific activity, improved temperature-independent stability, improved pH-independent stability, increased catalytic efficiency, increased hydrogen evolution rate.

5. The expression vector of claim 1, wherein the host is a hydrogenase-null microbial strain, and wherein the altered hydrogenase activity comprises restoration of detectable hydrogenase activity within the host.

6. The expression vector of claim 1, wherein the host is selected from E. coli strains: GW12, GW12A, GW1234, GW12AP, GW1234P, GW12APN, GW1234PN, GW0123HE and GW0123HEP.

7. The expression vector of claim 1, comprising a nucleic acid sequence derived from a microbial species selected from: R. eutropha, E. coli, and C. acetobutylicum.

8. The expression vector of claim 1, comprising a sequence of a gene or gene fragment, or a derivative thereof, selected from: hoxBC, hoxFUYH, hoxKGZ, hycEG, hydAEFG, hyp_{RE}, hoxMLOQRTV, and hoxWI.

9. The expression vector of claim 1, wherein the altered hydrogenase activity is associated with a condition selected from the group consisting of: increased enzyme yield, improved expression levels of hydrogenase, improved specific activity, improved temperature-independent stability, improved pH-independent stability, increased catalytic efficiency, increased hydrogen evolution rate, improved host compatibility, elevated cofactor levels, light energy-dependent ATP production.

10. (canceled)

11. The expression vector of claim 1, comprising at least one of: SEQ ID NO: 62, 66, 72-76, and 81.

12. A hydrogenase-null microorganism for heterologous expression of active hydrogenase.

13. The microorganism of claim 11, transformed with the expression vector of claim 1.

14. The microorganism of claim 11, wherein the microorganism is E. coli.

15. The microorganism of claim 11, wherein a portion of the genome is deleted, and wherein said portion comprises at least one sequence selected from: SEQ ID NO: 56, 57, 61, 63, 64 and 65.

16. The microorganism of claim 11 transformed with one or more expression vectors comprising at least one of: SEQ ID NO: 62, 66, 72-76 and 81.

17. The microorganism of claim 11 wherein one or more of SEQ ID NO: 62, 70-76, 81 and 82 is integrated into the genome of the microorganism.

18. A fuel cell system for oxidation of molecular hydrogen comprising the microorganism of claim 12.

19. A recombinant fuel-cell catalyst comprising at least one hydrogenase enzyme, wherein the hydrogenase enzyme is at least one of: a regulatory hydrogenase, a soluble hydrogenase, and a membrane-bound hydrogenase.

20. The catalyst of claim 18, wherein the hydrogenase is encoded by one or more of SEQ ID NO: 72, 73 and 74.

21. The catalyst of claim 18, comprising hydrogenase produced by expression of the expression vector of claim 1.

22. The catalyst of claim 18, comprising: hydrogenase expressed in the microorganism of claim 12.

23. A method of producing hydrogen, comprising:
providing the microorganism of claim 12;
growing the microorganism in a cell culture medium; and
recovering hydrogen produced by the microorganism.


SEQUENCE LISTING (83 sequences)

SEQ ID NO: 1 - DNA, 65 bp, Artificial Sequence
  Synthetic nucleic acid sequence
atgaataacgaggaaacattttaccaggccatgcggcgtcagggcgtgtaggctggagctgcttc

SEQ ID NO: 2 - DNA, 65 bp, Artificial Sequence
  Synthetic nucleic acid sequence
agctcgctaccgtcgtcgcccagcacgtgtgttgaacaggcgaggcatatgaatatcctccttag

SEQ ID NO: 3 - DNA, 65 bp, Artificial Sequence
  Synthetic nucleic acid sequence
tgaacagacgtaattttattaaagcagcctcctgcggggcattgcgtgtaggctggagctgcttc

SEQ ID NO: 4 - DNA, 65 bp, Artificial Sequence
  Synthetic nucleic acid sequence
ttacagaaccttcactgaaaccacttcgttgccgtcagcatccaccatatgaatatcctccttag

SEQ ID NO: 5 - DNA, 65 bp, Artificial Sequence
  Synthetic nucleic acid sequence
atgtctgaagaaaaattaggtcaacattatctcgccgcgctgaatgtgtaggctggagctgcttc

SEQ ID NO: 6 - DNA, 65 bp, Artificial Sequence
  Synthetic nucleic acid sequence
tcatcggatacgcgcctcttcaacaacatgattcagatggctgaccatatgaatatcctccttag

SEQ ID NO: 7 - DNA, 65 bp, Artificial Sequence
  Synthetic nucleic acid sequence
aacgttaattcatcgtcaaatcgtggcgaagcgattctcgccgccgtgtaggctggagctgcttc

SEQ ID NO: 8 - DNA, 65 bp, Artificial Sequence
  Synthetic nucleic acid sequence
tcagtcatgtaacccccggagtacgcgaaagagttgctgaacgatcatatgaatatcctccttag

SEQ ID NO: 9 - DNA, 65 bp, Artificial Sequence
  Synthetic nucleic acid sequence
aaaaaatgcttaaagctggcatctctgttaaacgggtaacctgacgtgtaggctggagctgcttc

SEQ ID NO: 10 - DNA, 64 bp, Artificial Sequence
  Synthetic nucleic acid sequence
acactcatcgacacgcccatccccaaacaggcgtaacgcctgcaaatatgaatatcctccttag

SEQ ID NO: 11 - DNA, 20 bp, Artificial Sequence
  Synthetic nucleic acid sequence
gcgacgtgcgccagtgcaga

SEQ ID NO: 12 - DNA, 20 bp, Artificial Sequence
  Synthetic nucleic acid sequence
cggcacgataaacagctcgc

SEQ ID NO: 13 - DNA, 20 bp, Artificial Sequence
  Synthetic nucleic acid sequence
gcgctattggtttgctcggc

SEQ ID NO: 14 - DNA, 20 bp, Artificial Sequence
  Synthetic nucleic acid sequence
agctgctatctcttcagtca

SEQ ID NO: 15 - DNA, 19 bp, Artificial Sequence
  Synthetic nucleic acid sequence
ggtggactgctgctggcgc

SEQ ID NO: 16 - DNA, 20 bp, Artificial Sequence
  Synthetic nucleic acid sequence
catgcagcatacttaacagc

SEQ ID NO: 17 - DNA, 23 bp, Artificial Sequence
  Synthetic nucleic acid sequence
tggctgcgcgggtgttaatggcg

SEQ ID NO: 18 - DNA, 23 bp, Artificial Sequence
  Synthetic nucleic acid sequence
gcttgctcctcttcgaccagtgc

SEQ ID NO: 19 - DNA, 20 bp, Artificial Sequence
  Synthetic nucleic acid sequence
tgcggtgaataatgtcgatg

SEQ ID NO: 20 - DNA, 20 bp, Artificial Sequence
  Synthetic nucleic acid sequence
catcgacgcgggtgatggcg

SEQ ID NO: 21 - DNA, 30 bp, Artificial Sequence
  Synthetic nucleic acid sequence
tctcgagctcatgtctgaagaaaaattagg

SEQ ID NO: 22 - DNA, 39 bp, Artificial Sequence
  Synthetic nucleic acid sequence
tcatcagatggcctctttcaatggcgattccttatttca

SEQ ID NO: 23 - DNA, 20 bp, Artificial Sequence
  Synthetic nucleic acid sequence
tgaaagaggccatctgatga

SEQ ID NO: 24 - DNA, 30 bp, Artificial Sequence
  Synthetic nucleic acid sequence
ctatgcatgccagttgactgaacaccacct

SEQ ID NO: 25 - DNA, 35 bp, Artificial Sequence
  Synthetic nucleic acid sequence
ccatggtaatgtctgacaagcaggccactgttctt

SEQ ID NO: 26 - DNA, 25 bp, Artificial Sequence
  Synthetic nucleic acid sequence
cagattctcggtcgccagcatcaag

SEQ ID NO: 27 - DNA, 25 bp, Artificial Sequence
  Synthetic nucleic acid sequence
cttgatgctggcgaccgagaatctg

SEQ ID NO: 28 - DNA, 32 bp, Artificial Sequence
  Synthetic nucleic acid sequence
tctagacggcaaccgatcaacgccggtctgcg

SEQ ID NO: 29 - DNA, 45 bp, Artificial Sequence
  Synthetic nucleic acid sequence
gaattcaggaggatcgtccatgcatgagctcagcctggcgggtgg

SEQ ID NO: 30 - DNA, 25 bp, Artificial Sequence
  Synthetic nucleic acid sequence
caatccggcttgcacgctggcgctc

SEQ ID NO: 31 - DNA, 25 bp, Artificial Sequence
  Synthetic nucleic acid sequence
gagcgccagcgtgcaagccggattg

SEQ ID NO: 32 - DNA, 32 bp, Artificial Sequence
  Synthetic nucleic acid sequence
aagcttagccgactcgctcaacaaatgcgcgg

SEQ ID NO: 33 - DNA, 18 bp, Artificial Sequence
  Synthetic nucleic acid sequence
atgaacgcgcccgctgag

SEQ ID NO: 34 - DNA, 26 bp, Artificial Sequence
  Synthetic nucleic acid sequence
gtcaagcttctaaccccgtcccctcc

SEQ ID NO: 35 - DNA, 22 bp, Artificial Sequence
  Synthetic nucleic acid sequence
atggtagttgcaatgggcattg

SEQ ID NO: 36 - DNA, 20 bp, Artificial Sequence
  Synthetic nucleic acid sequence
tcatgcatggacagtatctc

SEQ ID NO: 37 - DNA, 38 bp, Artificial Sequence
  Synthetic nucleic acid sequence
gagatactgtccatgcatgaatgaacgcgcccgctgag

SEQ ID NO: 38 - DNA, 31 bp, Artificial Sequence
  Synthetic nucleic acid sequence
agcccatggatggtcgaaacattttatgaag

SEQ ID NO: 39 - DNA, 29 bp, Artificial Sequence
  Synthetic nucleic acid sequence
agcaagctttcagcgaaaaagggaaccgt

SEQ ID NO: 40 - DNA, 29 bp, Artificial Sequence
  Synthetic nucleic acid sequence
agcccatggatggatagtcgtatcacgac

SEQ ID NO: 41 - DNA, 27 bp, Artificial Sequence
  Synthetic nucleic acid sequence
agcaagctttcagcgggcgcgttcatc

SEQ ID NO: 42 - DNA, 51 bp, Artificial Sequence
  Synthetic nucleic acid sequence
gaatgatataatggaaacatggtatggatccgccgggagcggatttgaacg

SEQ ID NO: 43 - DNA, 19 bp, Artificial Sequence
  Synthetic nucleic acid sequence
ccgggagcagacaagcccg

SEQ ID NO: 44 - DNA, 53 bp, Artificial Sequence
  Synthetic nucleic acid sequence
aattcggatccttttgctttttagctttaagaaatgatataatggaaacatcg

SEQ ID NO: 45 - DNA, 32 bp, Artificial Sequence
  Synthetic nucleic acid sequence
agcccatggtgaaaacaataatcttaaatggc

SEQ ID NO: 46 - DNA, 27 bp, Artificial Sequence
  Synthetic nucleic acid sequence
ttattcatgttttgaaacatttttatc

SEQ ID NO: 47 - DNA, 27 bp, Artificial Sequence
  Synthetic nucleic acid sequence
atggataatataataaagttaattaat

SEQ ID NO: 48 - DNA, 23 bp, Artificial Sequence
  Synthetic nucleic acid sequence
ttaaccaatagattctttgtagc

SEQ ID NO: 49 - DNA, 23 bp, Artificial Sequence
  Synthetic nucleic acid sequence
atgaatgaacttaactcaacacc

SEQ ID NO: 50 - DNA, 23 bp, Artificial Sequence
  Synthetic nucleic acid sequence
ttagttcctactcgattgattaa

SEQ ID NO: 51 - DNA, 34 bp, Artificial Sequence
  Synthetic nucleic acid sequence
agcagtactatgtataatgttaaatctaaagttg

SEQ ID NO: 52 - DNA, 24 bp, Artificial Sequence
  Synthetic nucleic acid sequence
ttagaatctaaaatctctttgtcc

SEQ ID NO: 53 - DNA, 54 bp, Artificial Sequence
  Synthetic nucleic acid sequence
attaattaactttattatattatccatttattcatgttttgaaacatttttatc

SEQ ID NO: 54 - DNA, 48 bp, Artificial Sequence
  Synthetic nucleic acid sequence
caactttagatttaacattatacatttagttcctactcgattgattaa

SEQ ID NO: 55 - DNA, 46 bp, Artificial Sequence
  Synthetic nucleic acid sequence
ggtgttgagttaagttcattcatttaaccaatagattctttgtagc

SEQ ID NO: 56 - DNA, 2909 bp, Escherichia coli
atgaataacgaggaaacattttaccaggccatgcggcgtcagggcgttacccggcgcagctttctcaaatattgtagtctggctgccacgtcgctgggattaggcgcgggaatggcaccaaagattgcctgggcgctggagaacaaaccgcgcattccggtggtatggatccacggtctggaatgcacctgctgtaccgaatcttttatccgctccgctcacccactggcgaaggacgtcatcctttccctgatttccctcgattacgacgatactttgatggctgccgccggaacccaggcggaagaagtctttgaagacatcatcacgcaatacaatggcaaatatatcctcgcagtagaaggtaatccgccgctgggcgagcaggggatgttctgtatcagcagcggtcgaccgtttattgagaaactcaaacgtgccgctgccggagccagcgcgattatcgcctggggaacctgcgcgtcctggggctgcgtgcaggccgcgcgacccaatccgacgcaggcaacgcctatcgacaaagtcatcaccgacaaacccattatcaaagtacctggctgcccgccgatcccggatgtgatgagcgccatcattacttacatggtgacctttgatcgcttgccagatgtcgacagaatgggccgtccgctgatgttctatggtcagcgaatccacgataaatgctatcgccgcgcccacttcgacgccggagagttcgtccagagttgggatgatgacgctgcccgcaaaggttactgcctgtacaaaatgggctgcaaagggcctaccacctataacgcctgttcctccacacgctggaatgatggcgtttctttcccaatccagtctggtcacggctgcctgggctgtgcggaaaatggtttctgggatcgcggttcgttctacagccgcgtggtcgatattccgcaaatgggtactcattccaccgccgataccgtcggtttaaccgcgcttggcgtggtggcagcggctgttggtgtgcacgcagtcgccagcgccgttgaccagcgcagacgtcataaccagcaacctacagaaaccgaacatcagccaggcaatgaggataaacaggcatgagcactcagtacgaaactcagggatacaccatcaataatgccggacgccgcctggtggtcgacccgattacgcgcatcgaaggccacatgcgctgcgaagtgaatattaacgatcagaatgtgatcaccaatgccgtctcctgcggcaccatctttcgcgggctggagatcatcctacaagggcgcgacccgcgcgatgcgcgggcgttcgttgaacgtatctgcggcgtctgtactggcgtacacgccctggcttcggtttacgccatcgaagatgctatcggtattaaagtgccggacaacgccaatatcatccgcaacattatgctggcaacgctctggtgccacgatcatctggtgcacttctatcagcttgccgggatggactggatcgatgtgttagatgcgctgaaagccgacccgcggaaaacctccgaactggcgcaaagtctctcctcttggccgaaatcatcccctggctatttcttcgacgtacaaaaccgcctgaaaaaatttgttgaaggcgggcagttggggatcttccgcaatggctactgggggcacccgcagtacaaactgccgccagaagctaacctgatgggctttgcccactatctcgaagctctcgatttccagcgtgaaattgtcaaaatccacgcggtctttggcggtaaaaacccgcatccaaactggattgtcggcgggatgccttgcgccatcaacattgacgaaagcggcgcggtcggggcagtcaatatggaacgcctgaacctggtgcagtcaattatcacccgcacggcggacttcattaacaacgtgatgatccccgacgccttagccatcggtcagttcaacaaaccgtggagcgaaatcggcactggtctttctgataaatgcgttctcagctacggcgcattcccggatattgccaacgactttggcgagaaaagtctgctgatgcctggcggcgcggtgattaacggcgacttcaacaatgtgctgccagtggatttggttgatccgcagcaggtgcaggagtttgtcgaccacgcctggtatcgatatcccaacgatcaggtcgggcgtcatccgttcgatggcatcaccgacccgtggtacaaccccggcgatgtcaaaggcagcgataccaacattcagcagctgaatgaacaggaacgctactcgtggatcaaagcgccacgctggcgcggtaacgcgatggaagtggggccgctggcgcgcacgttaatcgcttatcacaaaggcgatgctgcgaccgttgagtcggtcgatcgcatgatgtcggcgttgaacctgccgctttccggtatccagtcaacgttaggccgcattttgtgccgcgcgcacgaagcgcagtgggccgcaggtaagttgcagtatttcttcgacaagctgatgaccaacctgaaaaacggcaatctcgccactgcttccacggaaaaatgggaacctgcaacctggccgacagagtgccgtggtgtcggttttaccgaagcgccgcgcggggcgttaggccactgggccgccattcgcgatggcaagattgatctctaccagtgcgtggtgccgaccacctggaacgccagcccgcgcgatcccaaagggcagattggcgcttatgaagcggcgctgatgaacaccaaaatggcgatccccgagcaaccgctggagatcctgcgtactctgcacagctttgacccgtgcctcgcctgttcaacacacgtgctgggcgacgacggtagcgagctgatctccgtgcaggtgcgttaa

SEQ ID NO: 57 - DNA, 3855 bp, Escherichia coli
gtgaacagacgtaattttattaaagcagcctcctgcggggcattgctgacgggcgcgctgccgtctgtcagtcatgcggctgctgaaaaccgcccgccaattccgggatcgctggggatgttgtacgactcgaccttgtgcgtaggctgccaggcttgcgtcaccaagtgtcaggatatcaatttccctgaacgtaacccgcaaggggaacagacctggtcgaacaacgacaaactgtcgccgtataccaataacatcattcaggtgtggaccagcggcacaggggtcaacaaagaccaggaggagaacggctacgcgtacattaagaaacagtgtatgcactgcgtcgatccgaactgtgtctctgtgtgcccggtctctgcactgaaaaaagatccgaaaaccggcattgtccattacgacaaagatgtgtgcaccggctgccgttactgcatggtcgcctgtccgtacaacgtgccgaagtacgactacaacaacccgtttggtgcgctgcataagtgcgagctgtgcaaccagaaaggtgtggaacgtctcgataaaggcggtctacctggctgcgtagaagtgtgcccggcgggcgcggtgattttcggtacgcgtgaagagctgatggcggaggcgaaaaaacgtctggcgctgaagcctggcagcgaataccactatccgcgtcagacgctgaaatctggcgacacttacctgcatacggtgccgaaatattatccgcatctgtacggcgagaaagagggcggcggtactcaggttctggtactgacgggtgtgccttatgaaaatctcgacctgccgaaactggacgatctttctaccggtgcgcgttccgaaaatattcaacacaccctgtataaaggcatgatgctaccactggctgtgctggcgggcttaaccgtgctggttcgtcgcaacaccaaaaacgaccatcacgacggaggagacgatcatgagtcatgatccacaaccgctgggcggcaaaatcatcagtaaaccggtcatgatttttggaccgttaatcgtcatctgtatgctcctgattgtgaagcgtctggtgttcggtctgggctctgtctctgacctgaacggcggcttcccgtggggcgtgtggatcgcgtttgacctgctgattggcaccggctttgcctgtggcggctgggcgctggcgtgggcggtatacgtctttaaccgtgggcaataccatccgctggtgcgtccggcgctgttggcgagtctgtttggttactcactgggtggcttgtcgatcactatcgacgtgggtcgctactggaacctgccgtacttctacattccgggtcacttcaacgtgaactcggtactgttcgagacggcggtctgtatgaccatctatatcggcgtgatggcactggagtttgctccggcactgtttgaacgtctggggtggaaggtgtcgctacagcgactaaacaaggtgatgttcttcatcatcgcgctcggtgcgctgctgccgaccatgcaccagtcttcaatggggtcgctgatgatctcggcgggctacaaggtgcatccgttgtggcagagctatgaaatgttgccgctgttctcgctgctgacggcgttcatcatgggcttctcgattgtcatctttgaaggttcgctggtgcaggcgggtctgcgtggcaacggtccggatgaaaagagtctgtttgttaagctgaccaacaccatcagtgtgttgctggcgattttcatcgtgctgcgctttggcgagcttatctatcgcgacaagctgtcgttagcgtttgccggtgacttctactccgtgatgttctggattgaagtcctgctgatgctcttcccgctggtcgttctgcgtgtggcgaacgtgcgtaatgattcccgcatgctgttcctgtcagcactgagcgcactgttaggttgtgcaacctggcgtctgacctattcgctggtggcattcaacccgggcggcggttacgcctacttcccgacctgggaagaactgttgatttctattggttttgtggctattgagatttgcgcttacatcgtactcattcgtctactgccgatacttcctcctttaaaacaaaacgatcataatcgtcatgaggcgagcaaagcatgagccagagaattactattgatccggtaacccgtattgaggggcatttacgcatcgattgcgaaatcgaaaatggcgtcgtttcgaaagcatgggcttccggtaccatgtggcgcggcatggaagagatcgtgaaaaaccgcgatccgcgcgatgcatggatgattgtgcaacgtatctgtggcgtatgtactaccactcacgcgctgtcttccgttcgtgcggcagaaagtgcgctgaatatcgacgttccggttaacgcgcaatacatccgtaacatcattctggctgcgcacaccacgcatgaccatattgttcatttctatcagctttcggcgctggactgggtggacatcacttctgcactgcaagctgacccaaccaaagcctccgaaatgctgaaaggcgtttcgacctggcacctgaacagtccggaagagttcaccaaagttcagaacaagatcaaagatctggttgccagcggtcagttgggtattttcgctaatggctactggggtcatccggcgatgaaactgccgccggaagtgaacctgattgcggtagcgcactacctgcaagcgttggagtgccagcgtgacgctaaccgcgtcgtggcgctgctgggcggtaaaacgccgcacattcagaacctggcggtaggtggtgtcgcgaacccgatcaacctcgacggtttgggcgtgctgaaccttgagcgcctgatgtacatcaagtctttcatcgacaaactgagcgactttgttgagcaggtttataaggttgataccgcagttattgccgcgttctacccggaatggctgacacgcggtaaaggtgcggtgaactacctgagcgtgccggaattcccgaccgacagtaaaaacggcagcttcctgttcccgggcggctacattgagaatgcggatctgtcctcgtatcgtccgatcacttctcattccgatgaatacctgatcaaaggcattcaggaaagcgcgaagcactcctggtataaagacgaagcgccgcaggcaccgtgggaaggcaccaccattccggcttatgatggttggtctgacgacgggaaatattcctgggtgaaatcaccgactttctacggcaaaacggtagaagtggggccactggctaatatgctggtgaaactggcggcaggtcgcgaatctacccagaacaaactgaatgaaatcgttgcgatttatcagaaactgactggcaacacgctggaagtggcacagctgcactccacgctgggccgtattattggtcgtaccgttcactgctgtgaattgcaggatatcctgcaaaaccaatacagtgcactgatcaccaatatcggcaaaggcgatcacaccacctttgtgaagccgaacattccggcaacgggtgaattcaaaggtgttggcttcctcgaagcgccgcgcggtatgctctctcactggatggttattaaagacggtatcatcagcaactaccaggcggttgttccatcaacctggaactctggtccgcgtaacttcaatgatgacgtcggtccttacgagcagtcgctggtgggtacaccggttgccgatccgaataaaccgctggaagtggtgcgtaccattcactcctttgacccgtgcatggcctgtgcggtacacgtagtggatgctgacggcaacgaagtggtttcagtgaaggttctgtaa

SEQ ID NO: 58 - DNA, 588 bp, Escherichia coli
atgagcgagcaacgcgtggtggtcatggggctgggcaacctgctgtgggccgatgaaggcttcggcgtgcgggtggcggaacggctgtatgcccattaccactggcccgagtatgtggagattgtcgatggcggtactcagggactgaacttgctggggtatgtcgaaagcgccagccatctgttgattctcgatgccattgactacgggctggaacctggaacgctgcgaacctatgccggagaacgcattccggcttatctcagcgcgaagaaaatgagcctgcatcagaacagtttctccgaagtgttggcgctggcggatatccgcggacatctgccagcacatattgccctcgtcggtctgcaacccgcaatgctcgacgactacggcggtagcctgagcgaactggcacgggagcaactgcccgctgcggaacaggcggcgctggcgcagcttgctgcgtggggaattgtgccgcaaccggctaatgaatcgcgctgtctcaattatgactgtctgtcgatggaaaattacgaaggcgttcgcttgcgccagtaccggatgacacaggaggagcagggatga

SEQ ID NO: 59 - DNA, 858 bp, Escherichia coli
atgagcgaaacttttttccatctgctggggccaggaacgcaaccgaacgatgacagtttcagcatgaatccactgccgatcacctgtcaggtgaatgatgaaccgagtatggcggccctggagcaatgtgctcacagcccgcaggtgattgcgctgttaaacgagttacaacatcaactaagcgaacgccaaccgccgttgggcgaggtgctggcagtcgatctgttaaatctcaacgccgacgatcgtcactttatcaatacgcttctcggggaaggggaagtgtcagtgcgcattcagcaggctgacgacagtgaaagtgaaatacaggaggcgatcttctgcggattatggcgggtgcgcagacgtcgcggcgaaaagttgctggaggacaaactggaggctggctgcgcgccgctggcgttgtggcaggcggcaacgcaaaatctcttgccgacagattcgctgttaccgccgcccattgatggcctgatgaatggcctaccgttggcgcatgagttactggcacatgtacgtaaccccgacgcgcagccgcacagcattaatctgacgcaattacccatcagcgaggctgatcggctttttctctcacgtctctgtgggccgggaaatattcagattcgtaccattggctatggcgagagctatatcaacgccacggggttacgccatgtctggcatttacgctgtacggacaccttaaaaggcccgttactggaaagttatgaaatctgcccaataccggaagtggtgctggcagcgccagaagatttggtcgactctgcgcagcggcttagcgaggtatgtcagtggctggcggaagctgcaccgacgtaa

SEQ ID NO: 60 - DNA, 1118 bp, Escherichia coli
atgactggagataacaccctcatccattctcacggcattaaccgtcgtgatttcatgaagctttgtgcagcattagccgccaccatggggttaagtagcaaagccgctgcagagatggccgaatcggttactaacccgcagcgtccgccagttatctggattggcgcgcaggagtgcaccggttgtacggaatctctgcttcgtgcaacgcatccaacggtagaaaacctcgtactggagactatctctctggagtatcacgaagtgctttccgccgccttcggtcatcaggtcgaagagaacaaacataacgctctcgagaagtacaaagggcagtatgtgttagtggtggatggttccatcccattaaaagataacggtatttattgcatggttgccggtgagccgattgtggatcacatccgcaaagcggcggaaggcgcagcagccattatcgctatcggttcctgctctgcgtggggcggtgttgccgcagctggagttaacccaactggcgcagtcagcctgcaagaagttctgccaggcaaaaccgttatcaatattccgggctgcccgccgaacccgcacaacttcctcgcgaccgttgcgcacatcatcacttacggcaaaccgccgaactggatgacaaaaaccgtccgaccttcgcctatggccgtctgattcacgaacactgcgaacgtcgcccgcacttcgatgctggtcgttttgccaaagagttcggtgatgaaggccaccgcgaaggctggtgcctgtaccacctcggctgtaaagggccagaaacttacggcaactgctcaacgctgcaattctgcgatgttggcggtgtgtggccggtggcgattggtcacccttgctatggctgtaacgaagaaggtatcggcttccataaaggcatccatcagcttgccaacgtcgaaaatcaaactccgcgttcacagaaaccggatgttaacgctaaagagggcggcaacgtctctgcaggcgctattggtttgctcggcggtgtggttgggttggttgccggtgtcagcgtgatggcggtgcgtgaactgggtcgtcagcaaaagaaagataacgctgactcacggggagaataa

SEQ ID NO: 61 - DNA, 462 bp, Escherichia coli
atgactatttgggaaataagcgagaaagccgattacatcgcacagcggcatcgtcgcctacaggaccagtggcacatctactgcaattcgctggttcaggggatcacgttatcgaaagcgcgcctgcatcacgccatgagctgcgcgccggacaaagaactctgtttcgtcctttttgaacattttcgcatttacgtcaccctggcggatggctttaacagccacaccatcgagtattacgtcgaaacaaaagatggcgaagacaaacagcggattgcgcaggcgcaactgagcattgacggcatgattgatggcaaggtcaacatccgcgatcgcgaacaggttctggaacactatctcgaaaaaatcgctggcgtttacgacagcttatacaccgctattgaaaacaatgtgccggtgaatttaagccaactggtaaagggacaaagcccggcagcatga

SEQ ID NO: 62 - DNA, 3029 bp, Artificial Sequence
  Synthetic nucleic acid sequence cloned from Escherichia coli
atgtctgaagaaaaattaggtcaacattatctcgccgcgctgaatgaggcatttccgggcgtcgtgctggaccacgcctggcagaccaaagatcagctgactgtcaccgtaaaggtgaactacctgccggaagtggtggagtttctttactacaaacagggtggctggctgtcggtgctgtttggtaacgacgaacgcaaactgaatggtcattacgccgtttactacgtgctgtcgatggagaagggcactaagtgttggattacggttcgcgtcgaagttgacgccaacaaaccggaatatccgtccgtgacgccgcgcgttccggcggcggtgtggggcgagcgtgaagtgcgcgatatgtacggtttgattccggttggtctgccggatgaacgtcgtctggtgctgccggatgactggccggatgaactttatccgctgcgtaaagacagcatggattatcgtcagcgtccggcaccgaccaccgatgctgaaacctacgagttcatcaacgaactgggcgacaagaaaaacaacgtcgtgccgattggtccgctgcacgtcacttctgatgaaccgggccacttccgtctgttcgtcgatggcgaaaacattatcgacgccgactaccgtctgttctacgtccatcgcggcatggaaaaactggcggaaacccgtatgggttataacgaagtgaccttcctctctgaccgtgtgtgcgggatctgcggctttgcccacagcaccgcctacaccacgtcggtggaaaacgcgatgggtattcaggtgccagaacgtgcgcagatgatccgcgccattctgctggaggtagaacgcttgcactcgcatctgctcaaccttggcctggcctgtcactttaccggcttcgactccggctttatgcagttcttccgcgtgcgtgaaacctccatgaaaatggcagagatccttaccggtgcgcgtaaaacctacggcctgaacttgatcggcgggattcgtcgcgatctgctgaaagacgacatgatccagacccgccagctggcacaacagatgcgtcgtgaagtgcaggagctggtggatgtgctgctgagcactccgaacatggaacagcgcactgtcggcattggtcgtctggacccggaaatcgctcgcgacttcagtaacgtcggcccgatggtccgtgccagcggtcacgcccgtgatacccgcgccgatcacccgtttgtcggctatggcctgctgccaatggaagtccacagcgagcagggctgcgacgttatttcccgtctgaaagtgcgtatcaacgaagtctataccgcgctgaacatgatcgactacggtctggataacctgccgggtggcccactgatggtggaaggctttacctacattccgcaccgctttgcgctgggctttgccgaagcgccgcgcggcgatgatatccactggagcatgaccggcgacaaccagaagctgtaccgctggcgctgccgtgccgcgacctacgcgaactggccgaccctgcgctacatgctgcgcggcaacaccgtttccgatgcgccgctgattatcggtagcctcgacccttgctactcctgtaccgaccgcatgaccgtggtcgatgtgcgtaagaagaagagcaaagtggtgccgtacaaagaactcgagcgttacagcattgagcgtaaaaactcgccgctgaaataaggaatcgccatgtttacctttatcaaaaaagtcatcaaaaccggcacggcgacctcgtcttatccgctggagccgattgcggttgataaaaacttccgtggtaagccagagcagaacccgcagcagtgcatcggctgcgcggcctgcgtcaatgcctgcccgtcaaacgccttaacggttgaaactgacctcgccacaggagagcttgcctgggagtttaatcttgggcactgcatcttctgtggacgctgcgaagaagtctgcccgacggcggcgatcaaactgtcgcaagagtacgaactggcggtgtggaagaaagaagacttcctgcaacagtcccgcttcgcgctgtgcaactgccgcgtctgcaatcgtcctttcgccgtccagaaagagatcgactacgccattgcgctgcttaagcacaacggcgacagccgcgcggaaaaccaccgcgaaagctttgagacttgcccggaatgtaagcgccagaaatgcctggtgccgtccgaccgtattgaactgactcgccatatgaaagaggccatctgatgagcaatttattaggcccccgtgacgccaacggcattccggtccccatgacggtggatgaatccatcgccagcatgaaggcgtcgttactgaaaaaaatcaaacgttctgcctatgtttaccgcgtggactgcggcggctgcaacggttgcgaaatcgaaattttcggcacgctttcgccgctgtttgatgcagaacgcttcggcattaaagtcgttccttcaccgcgtcatgcggatattttactgtttaccggcgcggtcacccgtgcaatgcgatcccctgcgctgcgtgcgtggcagtccgcgccggacccgaaaatttgtatctcctacggtgcctgcggtaacagtggcgggatcttccacgatctctactgcgtgtggggcggtacggataaaattgtccctgtggatgtttatatccctggctgcccgccaacgcctgccgccacgctgtacggctttgcaatggcgctcggcctgctggagcagaaaattcacgcccgtgggccgggtgaactggatgaacaaccggcggagatcctgcatggtgatatggtgcagccgctgcgcgtgaaagtggatcgcgaagcacgtcgcctggcgggttatcgttacggtcgtcagattgccgatgattaccttacacagttagggcagggcgaagaacaggttgcacgctggctggaagcggaaaacgatccgcgtctgaacgagattgtcagccatctgaatcatgttgttgaagaggcgcgtatccgatga

SEQ ID NO: 63 - DNA, 3029 bp, Escherichia coli; identical to SEQ ID NO: 62

SEQ ID NO: 64 - DNA, 2978 bp, Escherichia coli
gtgaacgttaattcatcgtcaaatcgtggcgaagcgattctcgccgccctgaaaacgcagttccccggcgcggtgctggatgaagagcgacaaacgcctgaacaggtcaccattacggtgaaaatcaatctgctgcctgacgttgtacagtatctttattatcaacatgatggctggcttccggtcctgtttggcaacgacgagcggacacttaacggtcattacgcggtttattatgccctttcaatggaaggggccgaaaaatgctggattgtggtgaaggcgctggtcgatgccgacagtcgggagtttccgtcagtcacaccgcgcgtccctgccgcggtctggggcgagcgagaaattcgcgatatgtacgggctgattccggttggcctgccggatcagcgtcgcctggtgttgcccgatgactggccggaagatatgcatccgctgcgcaaagatgcgatggattatcgactgcgccctgaaccgacgactgattccgaaacgtatccgtttatcaatgagggcaacagcgatgcgcgggtgatccctgtcggcccgctgcatatcacctccgatgaaccgggtcacttccgcttgtttgtggatggcgagcaaattgtcgatgctgattaccgcctgttttatgtccatcgcggcatggagaaactggcagaaacgcggatgggctacaacgaagtgaccttcttatcggaccgcgtgtgtgggatttgcggttttgcccacagtgtggcctataccaattcggttgaaaatgcactggggattgaggtgccgcaacgagcacatactattcgctcgattctgctggaagtcgaacggctacacagtcatttgcttaaccttggcctctcctgccatttcgttggttttgataccggctttatgcaatttttccgcgtgcgggaaaagtcgatgacgatggcggaattgctgatcgggtcgcgtaaaacctacggtctgaatctgattggtggtgttcgccgcgatattctcaaagagcaacgtctgcaaacgctgaaactggtgcgcgagatgcgcgccgacgtgtcggagctggtagagatgctgcttgctacgccgaatatggaacaacgcactcagggcattggcattctcgaccgacaaatcgcccgtgatttgcgctttgatcacccctacgccgactacggcaatattccaaaaacactgtttacctttaccggcggcgatgttttctcccgcgtgatggtccgtgtcaaagagacgtttgattcgctggcaatgctggaatttgccctcgacaacatgccggataccccactgctgaccgaaggctttagctataaacctcacgcattcgcgctgggctttgttgaagcgccacgcggtgaagacgtgcactggagcatgctcggtgataaccaaaaattgttccgctggcgctgccgtgccgccacctacgccaactggccggtgttgcgttacatgctgcgcggcaataccgtttctgacgcaccgctgattatcggtagccttgatccctgctactcctgtaccgaccgtgtgacgctggtagatgtgcgcaagcgccagtcaaaaaccgtgccgtataaagagatcgaacgctacggcattgatcgtaaccgttcgccgctgaagtaaggacagaagatgctgaagttactgaaaactattatgcgcgccggaaccgcgacggtgaaatatcccttcgcgccactggaggtcagccctggctttcgcggaaaaccggacctgatgcccagccaatgtattgcctgcggtgcctgcgcctgtgcttgtccggcaaatgcgctgactatccagaccgacgaccagcaaaattcgcgcacctggcagctctatctggggcgttgtatttactgcggacgttgtgaagaagtgtgcccgaccagagccatccagcttaccaataactttgaactgaccgtcaccaataaagccgatctctatacccgcgcgacgttccatctacaacgttgcagccgttgcgaacgcccgtttgccccgcaaaaaaccatcgcactggctgctgaattgttagcacagcaacaaaatgcgccacaaaaccgcgaaatgttgtgggcgcaagcgagcgtctgcccggaatgcaaacaacgcgcgacgctgatcaacgacgatacagatgtactgctggtggctaaggagcagctatgagtccagtgcttacacaacatgtcagccagcccatcacgctggacgagcaaacgcaaaagatgaagcggcatttgctacaggatatccgtcgctcggcttacgtttatcgcgtcgattgcggcggctgcaacgcctgtgaaatcgaaatttttgctgccattacaccagtattcgacgcagaacgttttggcattaaggttgtttcatcaccgcgtcacgccgatattttgttatttactggcgcagtcacccgggcgatgcgtatgcctgcacttcgggcgtatgagtctgcccccgatcataaaatttgtgtttcctacggcgcgtgcggtgtcggcggcggtattttccacgatctctacagcgtctggggcggtagcgacaccattgtccccattgatgtttggatccccggctgcccgccaacaccggccgccaccattcacggtttcgccgtggcgctcggtttgctgcaacagaagattcacgctgtggattatcgcgatcccaccggggtgactatgcaaccgttgtggccgcagatcccgccatcacagcgtatcgccattgagcgagaagcgcggcggctggcgggctatcgtcaggggcgagaaatttgcgatcggctcctgcgccatttaagcgacgatcctacaggaaatcgggttaacacctggttgcgcgatgccgacgatccacgtctcaatagtatcgttcagcaactctttcgcgtactccgggggttacatgactga

SEQ ID NO: 65 - DNA, 2622 bp, Escherichia coli
ctgctcaacaaagttgacctgttgccgtatctcaactttgacgttgaaaagtgcatcgcctgcgcccgcgaagtcaatccagaaattgaaatcatccttatttccgccaccagcggcgaagggatggaccagtggctgaactggctggagacacagcgatgtgcataggcgttcccggccagatccgcaccattgacggcaaccaggcgaaagtcgacgtctgcggcattcagcgcgatgtcgatttaacgttagtcggcagctgcgatgaaaacggtcagccgcgcgtgggccagtgggtactggtacacgttggctttgccatgagcgtaattaatgaagccgaagcacgcgacactctcgacgccttacaaaacatgtttgacgttgagccggatgtcggcgcgctgttgtatggcgaggaaaaataatgcgttttgttgatgaatatcgcgcgccggaacaggtgatgcagttaattgagcatctgcgcgaacgtgcttcacatctctcttacaccgccgaacgccctctgcggattatggaagtgtgtggcggtcatacccacgctatctttaaattcggcctcgaccagttactgccggaaaacgttgagtttatccacggtccggggtgcccggtgtgcgtactgccgatgggtagaatcgacacctgcgtggagattgccagccatccggaagtcatcttctgtacctttggcgacgccatgcgcgtgccggggaaacagggatcgctgttgcaggcaaaagcacgcggtgccgatgtgcgcatcgtttactcgccgatggatgcgttgaaactggcgcaggagaatccaacccgcaaagtggtgttcttcggcttaggttttgaaaccactatgccgaccaccgctatcactctgcaacaggcgaaagcgcgtgatgtgcagaatttttacttcttctgccagcacattacgcttatcccgacgttgcgcagtttgctggaacagccggataacggtatcgatgcgttcctcgcgccgggtcacgtcagtatggttatcggcaccgacgcctataattttatcgccagcgattttcatcgtccgctggtggttgctggattcgaaccccttgatctactacaaggcgtggtcatgctggtgcagcagaaaatagcggcccacagcaaggtagagaatcagtatcgtcgagtggtaccggatgccggtaacctgctggcgcaacaggcgattgccgatgtgttctgtgtcaacggcgacagcgaatggcgcggcttaggcgtgattgaatcttctggcgtgcacctgacgccggattatcaacgattcgatgccgaagcacatttccgcccggcaccgcagcaggtctgcgatgacccgcgcgcgcgttgtggtgaggtattaacgggcaaatgtaagccgcatcaatgcccgctgtttggtaacacctgtaatcctcaaaccgcgtttggtgcgctgatggtttcctccgaaggagcgtgcgccgcgtggtatcagtatcgtcagcaggagagtgaagcgtgaataatatccaactcgcccacggtagcggcggccaggcgatgcagcaattaatcaacagcctgtttatggaagcctttgccaacccgtggctggcagagcaggaagatcaggcacgtcttgatctggcgcagctggtagcggaaggcgaccgtctggcgttctccaccgacagttacgttattgacccgctgttcttccctggcggtaatatcggcaagctggcgatttgcggcacagccaatgacgttgcggtcagtggcgctattccgcgctatctctcctgtggctttatcctcgaagaaggattgccgatggagacactgaaagccgtagtgaccagcatggcagaaaccgcccgcgcggcaggcattgccatcgttactggcgatactaaagtggtgcagcgcggcgcggtagataaactgtttatcaacaccgctggcatgggcgcaattccggcgaatattcactggggcgcacagacgctaaccgcaggcgatgtattgctggtgagcggtacactcggcgaccacggggcgactatccttaacctgcgtgagcagctggggctggatggcgaactggtcagcgactgcgcggtgctgacgccgcttattcagacgctgcgtgacattcccggcgtgaaagcgctgcgtgatgccacccgtggtggtgtaaacgcggtggttcatgagttcgcggcagcctgcggttgtggtattgaactttcagaagcggcactgcctgttaaacctgccgtgcgtggcgtttgcgaattgctgggactggacgccctgaactttgccaacgaaggcaaactagtaatagctgttgaacgcaacgcggcagagcaagtgctggcagcgttacattcccatccactggggaaagacgcggcgctgattggtgaagtggtggaacgtaaaggtgttcgtcttgccggtctgtatggcgtgaaacgaaccctcgatttaccacacgccgaaccgcttccgcgtatatgctaataaaattctaaatctcctatagttagtcaatgaccttttgcaccgctttgcggtgctttcctg

SEQ ID NO: 66 - DNA, 6950 bp, Artificial Sequence
  Synthetic nucleic acid sequence cloned from Ralstonia eutropha
atgcatgagctcagcctggcgggtggcatcttgcagttgctcgaagacgctgcgcggcgcgaacgatttgctcgggtcacgctcttgcgcctcgaggcaggcaagttgtgtggcgtggaggtacgcgcgctgcgtttcgcgctcgaggccattgcttcgggaacctgcctggagggcgcttgtatcgagatcgaggaacctgagggacaggcctggtgcctgcagtgcaatgcagcggttgctttggcggagcgtggcgcaccatgcagcggatgcggcggctaccggctccagcccactgcggggaccgagttgcgtgttatggacatgctggtcgaagatcactggtgtcctgaatgaaaaattcattgaatcaggacaccagggtgaagcgatctgcgagaagaaaggagtaggcgaaaatgtgcgtcatatgtggatgcaataccaatcacgagactgcccggcaagacgagaataagggcgaacatgctcccggccgtggcctcgtcgataccggcaccgagccgccgggcgcggcgcatgtacggatcgatgcgtccaccggtgatctgcactacggcgcggggccggcgcacgtgtctgtgccgggtctcagtcaagcgcgtgcgatcaaactggaacaggacgtgctgggtcacaacaatcgccttgctgcacacaaccgtcagcatttcgtcgcacatggtgtgcttgcactgaatctcgtttccagtccggggtcgggcaagacgaccttgctctgcactaccatcgaagccttgcggcggtgccgtgcggacctgcagctggccgtgatcgagggcgaccagcagaccagtcatgacgcagagcgcatccgggccacgggtgtacctgcgatccagatcaataccggcaagggctgtcatctcgatgcgctaatggttgccaatgcgtacgagcgcctccccttgcacgccgcggcacatgcgcacacccatgagcaccgtcaagaggacggacagcattcccaccatcatgaccacgagcatgctcgccacgatcaccacgatcaccgatccagcggcatcggcagcgtcttgttcatagaaaacgtcggcaatctggtgtgccctgcaatgtgggatctgggcgaatccgcgaaggtggcgatcctgtcggtgaccgagggcgaggacaagccgctcaagtatcccgacatgttcgccgccgccagcctgatgatcctgaacaagatcgacctgctgccacatctgcgcttcgatgtagcacgctgcatcgaatatgcgcggcaagtcaatccgcaccttcaggtgctgcagctgtcggccgcgacaggagagggcgtggacgagtggctagactggatgctggccggcggcgcggcggcaggggctgctgccgcaacggatggcgcgcgcatgcgtatcgccgcgccggaggccggaatcgctgccctcggaacgcagttggctcccggtccggcggtgtcgaaggagatgtgagatgcgagcgcagaccttgccagccggcagcgaccatgcgccggtgctggcttgcggtgcatggctgaagaatgccgcatgcctgttgcggggcgccgaggtcctctggtccccgatacatggcgatcttggagatccggccaactgcgatgcgcttgaccagtcggtcgaacagttacttgacagcgctcacggccaggtgcaggcggtggcgcatgatctgcacccggatttctatagcacgcaactcgcgcaacgcctggcggctcggctttgcgtgccggcagtggccgtccagcaccatcacgcgcacatcgccgcgctgatggcggaatacgatctgagagaacccgtgatcggcttggcgctggacggcgtcggtcttggcacggatggtacggcttggggtggggagttgctatgggtttctccgtcagagtggtgtcgcttggggcatctccagtcgctgccgctgcctggcggcgacgtggcagcgcgcgaaccctggcgcatggcagccgcagcgctacatgtgctggaccgtacgggcgagatcgggcggcgctacggcgccgttgtaggtgagcaggcggcccgtaccgtggcagcgatgctggaacggcaactcaactgtccacgatccagtagcgccgggcgctggttcgacgccgcggcgggtgcgctgggtgtcagtgtgcggcagcaggccgaggcgcaggctgcgatcgccctggaggcgcttgcggccgactatctgtccgcgctgtcgccgcctgaatgcgtcggtacgtacgtcgtggatcaggacggagttctggatctgcgtgggctgctggagcagctgtttgcgctggccgatgagggtcaggcggggcaagcggcgcggggcgcggcgctgttccatgtggcgctcgccgaggctctggtggggtgggccgctgacgcagcgcagggccacggactgaagaccgtggccctgggcggtggatgtttcatgaatggcattttgagcgccagcgtgcaagccggattggcagcgcgaggtttgcaagcgttgctgccgcgcgcggtatcgtgcggcgatgcagggctcgcgctggggcaagcctgggtcgcggcccgccagcctacggctgccctggcgccgcaaacgcatctacaggaggagggcgcgccatgtgcctagcgattcccgcacgtttggtggaactgcaagcggaccagcagggcgtagtcgacctgagcggtgtacgtaagaccatatctctcgcgctgatggccgatgccgtagtcggtgactacgtcatcgtgcatgtcggctacgcgattggcaagatcgatccagaggaagcagaacgcacgctgcgtctgttcgcggaattggagcgagtgcagccgcctgcgtccgagccgatgcatgggatgaacattcatcaggagccggcatgaaatacatcgaagaatttcgcgacggcgagctggcgcaacgcattgcggcgcacgttcgcgctgaggcgcggccggggcaacgctacaacttcatggagttctgcggcggacacacgcatgccatttcgcgttacggcgtgaccgaactgctgcccgagaacgtgcggatgattcacgggccgggctgcccagtgtgcgtgctgccgatcggccgcattgacctagcgctgcatctggcgctggagcgcgacgccatcgtctgcacttacggcgacacgatgcgggtgccggcctcggggggcatgtcgttgattcgggccaaggcgcacggggccgacatccgcatggtctactcggccgctgatgcgctgaaaattgcgcagcggcatccacagcgcgaggtggtgttccttgccattggcttcgagactacaacgcctccaactgcgctgattattcgcgaggcgaaggcgcgccaggtggataacttcagtgtgttgtgttgccacgtgcttacgccgtcggccattacccacatcctggagtcgcccgaggtgcgcgactatggcactgtgcccatcgacggattcgttgggccggctcatgtgagtatcgtgatcggtacccggccctatgagcatttttcacgcgagtacggtaagccggtggtgatcgcaggcttcgagccgctcgatgtgatgcaagccattctgatgttggtacgacaagtcaacagcggacgcgcagaggtggaaaatgagttcgtgcgcgctgtcacccgcgatggcaacgagagcgcgcaggccatggtgtcggaagtgttcgagctgcggccgtctttcgagtggcgaggactcggcgaggtgccatatagcgccttgcgcatccgcgcgcagtttgcgcgtttcgacgctgagcaacgcttcgatctgcgctaccggccggtgcctgacaataaggcttgcgaatgcggcgcgatcttgcgtggcgtcaagaagccaaccgactgcaagctgttcgctaccgtctgcaccccggagaatccgatggggtcgtgcatggtatccagcgaaggtgcctgcgccgcgcactattcgtatgggcggttcaaggatatccccttggtggcagcatgagcggcaccgttaaactgggctatcaacggcccctgaacatcaagagcggcagaatcgacatgggccacggcgcgggggggcgcgcagccgcacaacttatccaggaactgttcgttgctgcgtttgacaatgagtggctgcgccagggcaacgaccaggcggctttcgccatgcctgccggggccagaatggtgatggccaccgacgcgcacgtggtgtcgccgctattcttccccgggggtgacatcggtagcctgtcggtgcacggcaccattaacgatgtggcgatggccggtgccaaaccgctgtatctggccgcgagtttcatcctcgaggaagggtttccgctagcggatcttaagcgcattgtcgaatcgatggccggggctgcgcgtgaggctggcgtgcctatcgtgacgggtgacacgaaagtggtcgagcaaggcaagggtgatggcgtattcattaccactaccggcgtcggcgtggtgccagcgggcattctaatcgacggcgccggggccaggcccggcgacgctattctgctctctggcactatgggggagcatggcgtagcgatcctgtccaaacgcgagtcgcttgaatttgacactgagatccgctcggacagcgccgcgctgcacgacttggtggcacagatgctggccgtggtgccgggggtacgagtgctgcgggatcctacgcgcggcggactggcgaccacgctcaacgaaatttccagtcaatcgggagtgggcatggtgttggacgaagcggcgatccccgtcctgccacaggtggacgcggcatgcgagctgctcgggcttgatccgctgtacgtggcgaacgaaggtaaattggttgccatttgcgcagctgcggacgcggatgccctgcttgccgccatgcgggggcatccgctcggtcgcgaggcacggcgcatcggagaggtcatcgaggacgggcgccactttgtgcagatgcgcacaaaattcggcgggatgcgcgtggtggactggctgtccggcgagcagcttccgcgcatttgttgagcgagtcggctatgcgcatattgctcctcacccatagcttcaacagtctgacgcagcgactgtttgtcgagttgcgccagcgcggccacctggtgtcggtggagttcgatattgccgattccgtgaccgaggaggccgtggcgctgttcgcccccgatctggtcatcgccccttttctcaagcgtgcgattccggaacgaatatggagtcgactggtctgtctggtggtgcatcctggcattgtcggcgatcgcggcccgagtgcgctggactgggcgatcgttcgagacgaacgaagttggggtgtgacggtgttgcaggccaacggcgagatggatgcaggtccggtatgggctagcgccacgttcccaatgcgcgcggcacgcaagagcagcctgtatcgtaacgaggtgacggtggcagcagtacaagctgtgctggaggcgctggcggcttttgaagccggctggcgttcggcgaatgatgcgcagagcggcccgggcacctggaatcctgcaatgcggcaggctgagcgcggcatcgactgggcgcgtgagtcgactgccgccgtgctggcaaagctacatgcggccgatagctttcccggcgtaccggatgccttgttcggccagccgtgccggctgttcgacgcccacgccgcgaccccgcagacggtcgcacgcgcgccgcgaggccagcctggcgatatcgttgcgcgccgtgagcatgccgtgctgcgcctgacggtggacgcaggcgtgtggatcggtcacgccaagctggccgtgcgcgatgagtgggaaaacacaagcctgccatctctcaagctgccggtggcgcaagtatttcatgaggcgtgggcgcaattgccggacctgtcgctgccgctggaacacgacgccggcgagtggagcgagttccggtactgggaagacgattccggcgcgggcctggtcggatacctcgccttcgactgctataacggggcgatgtcgaccgcgcaatgccaacggctttgcgccgccttgcgctacgcgcgcggtcgccagacgcgggtgctggtgctgctggggggagaggattttttcagcaatggtattcatctgcaccaaatcgaggcggcggagcaccggggcgcggagagcgcggccgatgcctcctggcgcaatattcaggcgatggacgacgtcgcgctggaaatccttgcgttttccgatcgcatgacggtgtcggcgctgcgcggcaatgcgggagcgggcggcgtgttcttggcgctggcggccgatcaggtatgggcgcgcgaaggtgtactcgtgaatccgcactacaagaacatgggcaatctgtatggttcggaatactggacctatctgttgccgcaacgcgtcggtgcgcaacgagccagtgatctgatggacggccgtttgccaatgtcggtccggcgggcgatcgagattgggctgatcgatgccagcttggacggcgacgcgcgctcgtgccttgccgagattggccgccgcgccgttgcgctggcccgggcgccagactatgctgcgcatattgacaacaagcggcgaaaacgggccgcagacgaagcggcaaagccgctggcccagtatcgagaggaagaattggtgcatatgcatcggaatttctatggattcgatccgagctatcacgtggcacgctatcatttcgtctacaaattaccccatgcacatacgcctcgccaccttgccatgcaccggggtggcagcctgccggcggcacaagagttgcaggcacttgcggggaaacgatcttga

SEQ ID NO: 67 - DNA, 855 bp, Escherichia coli
atgactgacaaaatgcaaagtttagctttagccccagttggcaacctggattcctacatccgggcagctaacgcgtggccgatgttgtcggctgacgaggagcgggcgctggctgaaaagctgcattaccatggcgatctggaagcagctaaaacgctgatcctgtctcacctgcggtttgttgttcatattgctcgtaattatgcgggctatggcctgccacaggcggatttgattcaggaaggtaacatcggcctgatgaaagcagtgcgccgtttcaacccggaagtgggtgtgcgcctggtctccttcgccgttcactggatcaaagcagagatccacgaatacgttctgcgtaactggcgtatcgtcaaagttgcgaccaccaaagcgcagcgcaaactgttcttcaacctgcgtaaaaccaagcagcgtctgggctggtttaaccaggatgaagtcgaaatggtggcccgtgaactgggcgtaaccagcaaagacgtacgtgagatggaatcacgtatggcggcacaggacatgacctttgacctgtcttccgacgacgattccgacagccagccgatggctccggtgctctatctgcaggataaatcatctaactttgccgacggcattgaagatgataactgggaagagcaggcggcaaaccgtctgaccgacgcgatgcagggtctggacgaacgcagccaggacatcatccgtgcgcgctggctggacgaagacaacaagtccacgttgcaggaactggctgaccgttacggcgtttccgctgagcgtgtacgccagctggaaaagaacgcgatgaaaaaattgcgtgctgccattgaagcgtaa

SEQ ID NO: 68 - DNA, 2355 bp, Escherichia coli
atgaatcctgagcgttctgaacgcattgaaatccccgtattgccgctgcgcgatgtggtggtttatccgcacatggtcatccccttatttgtcgggcgggaaaaatctatccgttgtctggaagcggcgatggaccatgataaaaaaattatgctggtcgcgcagaaagaagcttcaacggatgagccgggtgtaaacgatcttttcaccgtcgggaccgtggcctctatattgcagatgctgaaactgcctgacggcaccgtcaaagtgctggtcgaggggttacagcgcgcgcgtatttctgcgctctctgacaatggcgaacacttttctgcgaaggcggagtatctggagtcgccgaccattgatgagcgggaacaggaagtgctggtgcgtactgcaatcagccagttcgaaggctacatcaagctgaacaaaaaaatcccaccagaagtgctgacgtcgctgaatagcatcgacgatccggcgcgtctggcggataccattgctgcacatatgccgctgaaactggctgacaaacagtctgttctggagatgtccgacgttaacgaacgtctggaatatctgatggcaatgatggaatcggaaatcgatctgctgcaggttgagaaacgcattcgcaaccgcgttaaaaagcagatggagaaatcccagcgtgagtactatctgaacgagcaaatgaaagctattcagaaagaactcggtgaaatggacgacgcgccggacgaaaacgaagccctgaagcgcaaaatcgacgcggcgaagatgccgaaagaggcaaaagagaaagcggaagcagagttgcagaagctgaaaatgatgtctccgatgtcggcagaagcgaccgtagtgcgtggttatatcgactggatggtacaggtgccgtggaatgcgcgtagcaaggtcaaaaaagacctgcgtcaggcgcaggaaatccttgataccgaccattatggtctggagcgcgtgaaagatcgaatccttgagtatcttgcggttcaaagccgtgtcaacaaaatcaagggaccgatcctctgcctggtagggccgccgggggtaggtaaaacctctcttggtcagtccattgccaaagccaccgggcgtaaatatgtccgtatggcgctgggcggcgtgcgtgatgaagcggaaatccgtggtcaccgccgtacttacatcggttctatgccgggtaaactgatccagaaaatggcgaaagtgggcgtgaaaaacccgctgttcctgctcgatgagatcgacaaaatgtcttctgacatgcgtggcgatccggcctctgcactgcttgaagtgctggatccagagcagaacgtagcgttcagcgaccactacctggaagtggattacgatctcagcgacgtgatgtttgtcgcgacgtcgaactccatgaacattccggcaccgctgctggatcgtatggaagtgattcgcctctccggttataccgaagatgaaaaactgaacatcgccaaacgtcacctgctgccgaagcagattgaacgtaatgcactgaaaaaaggtgagctgaccgtcgacgatagcgccattatcggcattattcgttactacacccgtgaggcgggcgtgcgtggtctggagcgtgaaatctccaaactgtgtcgcaaagcggttaagcagttactgctcgataagtcattaaaacatatcgaaattaacggcgataacctgcatgactatctcggtgttcagcgtttcgactatggtcgcgcggataacgaaaaccgtgtcggtcaggtaaccggtctggcgtggacggaagtgggcggtgacttgctgaccattgaaaccgcatgtgttccgggtaaaggcaaactgacctataccggttcgctcggcgaagtgatgcaggagtccattcaggcggcgttaacggtggttcgtgcgcgtgcggaaaaactggggatcaaccctgatttttacgaaaaacgtgacatccacgtccacgtaccggaaggtgcgacgccgaaagatggtccgagtgccggtattgctatgtgcaccgcgctggtttcttgcctgaccggtaacccggttcgtgccgatgtggcaatgaccggtgagatcactctgcgtggtcaggtactgccgatcggtggtttgaaagaaaaactcctggcagcgcatcgcggcgggattaaaacagtgctaattccgttcgaaaataaacgcgatctggaagagattcctgacaacgtaattgccgatctggacattcatcctgtgaagcgcattgaggaagttctgactctggcgctgcaaaatgaaccgtctggtatgcaggttgtgactgcaaaatag

SEQ ID NO: 69 - DNA, 954 bp, Escherichia coli
atgcgggcgaaacttctgggaatagtcctgacaacccctattgcgatcagctcttttgcttctaccgagactttatcgtttactcctgacaacataaatgcggacattagtcttggaactctgagcggaaaaacaaaagagcgtgtttatctagccgaagaaggaggccgaaaagtcagtcaactcgactggaaattcaataacgctgcaattattaaaggtgcaattaattgggatttgatgccccagatatctatcggggctgctggctggacaactctcggcagccgaggtggcaatatggtcgatcaggactggatggattccagtaaccccggaacctggacggatgaaagtagacaccctgatacacaactcaattatgccaacgaatttgatctgaatatcaaaggctggctcctcaacgaacccaattaccgcctgggactcatggccggatatcaggaaagccgttatagctttacagccagaggtggttcctatatctacagttctgaggagggattcagagatgatatcggctccttcccgaatggagaaagagcaatcggctacaaacaacgttttaaaatgccctacattggcttgactggaagttatcgttatgaagattttgaactcggtggcacatttaaatacagcggctgggtggaatcatctgataacgatgaacactatgacccgggaaaaagaatcacttatcgcagtaaggtcaaagaccaaaattactattctgttgcagtcaatgcaggttattacgtcacacctaacgcaaaagtttatgttgaaggcgcatggaatcgggttacgaataaaaaaggtaatacttcactttatgatcacaataataacacttcagactacagcaaaaatggagcaggtatagaaaactataacttcatcactactgctggtcttaagtacacattttaa

SEQ ID NO: 70 - DNA, 6640 bp, Artificial Sequence
  Synthetic nucleic acid sequence cloned from BAC clone MED66A03
atgccggatagctattttataagtgaaaagcagcattcaccatccaataaatacaccagtgatggatcccataattttatatttaatcaattttttaattctatcctgccatcgaataaacaccccttatcagctgctgcaacccaccatttttcctctaagggaaagcaattaagagcaaagatagcgctaagtgcgggagaaaaatttggagcagatgatgctgcatgtcttcattgggcttccgcagtcgaattactacataatgcgtcattaattcatgatgatatttgtgatggagacactatacgcagaaataacgcatctgtttgggctaaatatggccgagatatagcgttggctttgggagactggatgatcgcagaagcttttcaacaggccgcaaaagccgctcatgtatcaaattcgtttaatctcgtaaccaaactttcaaatcacgttaaatccactattgctggacaagccctagaatttgacaatgcactatacccagatatagataaatatcttgagattagcgctggaaaaacagcaccattattcgttgccgccatcgaagggatagctgaaattgcagggagaagcgaattaatagaggaaattaaccactattttgtaacgatgggaatttgttatcaatttgcgaacgatattcaaaatattctcggaacagatggagcagcaagcccctcctcagatttaaaacgacgagcaccaaatgctgtaattgtatattttcgtaattttcttaaaagtccggaacggtctctattcgactcctggctagccggtaaatcacaaaataaagctaattattggcaaaagaaaattacggaatctgatgcgattaatgaagttgcaaaagaaatgtataaattgttaaaacgagcagaaggtgtttctggcactctcccaaaagaatgtattggtgttattgccccaatccaaaaacaacttcggaatgtttgtgaacaaattggtgcttcttgcccctaacctttgttattacctgcatcaatttatcgtattacaaaaaaccatttttttggcgtttgtaactacatattttacatataatttgtattcttaactcaaataaaataagaaatgacaaatataattatggtacaattttagtatgacacgttctttagttattggcgcaggacttggtggaatagcggccgccttgaggttacgcgcaaaaggagatgaggtagaggttttagaaagactacctgcggctggcggaagagctcaggtttttgagaaaaacggtttcaaacatgatgcgggaccaactgtgattactgccccgtttttatttgacgaattattcagcttgtttggtgaaaaaagacaagactacttagagttcaaacccttagatttgtggtatcgtttttactttgactcgaccggtgatacattcgattacactgctagtattgaaaaaactaaagaagaaatttctcgctttaacagagaagatacggaaggttatgataggttaattaatacatcggaaaagatctttaatattggttttaccaaacttgctgataaaccttttacaaagtttagctcaatgctcaaacaaataccttctttattgaaacttaaaagctacctttccgtatctcagctggttaatagtcatataaaacatcctcaaattcgccaagctttctcaattcaccccctattagtaggaggtaatccatttgacacaacatctatttacgcactcattcattatctagagcggaaatggggtgtatttttctgtatgggcggcactggtaaattgattaaggagctagaatcgttgatgcttcgacaaaatatttctatccgttttaatacagatgttgaacagattttggttgaaaatgggcgttcaactggggttgtaactagttcaggagaaataatttcttctaaccgaatagtgtgtaatgcggatacaccagctgtttatgatgagctattatctgcacaaccaagacgccggaaaaaggccattcctgaaaaatttactcgatattcaatgggcctatatgtattattttttggtacgaatacgaaatataaaaaagttgcccatcatacaatatggatgggaaagcgttaccgtgaactgctctcagatatattcaacgcaaaaatattaacagatgacttttccttgtaccttcacagaccaactgcaacagatccctcttttgctccggatgggtgcgatagcttttacgtgttgtgtcctgtacctaaccttcaaggtaacgtaaattgggacgtgcaaggacctgctttgcaggaaagaatcataaatgcactaaacagaaccataatgccaaatttgaaagaaacaattgtggacccattttggatgagcccacgtgaatttaagaccaactatagagcaaaacatggcgctggattttctattgcaccaatttttgcacaatcagcctggtttagatatcataacagagaccctcatattaataatttattctttactggcgctggcacacatccaggtgcgggtatgccaggtgttttatgttcagcaaaagttgttgaaaatttaattgcagaagaacaagtaaagcagtcaaaataaattgatttttgagggtttttgcagaaaaggataaaattagacgtgcttttggaagctgtaacacataaaacgtatgagggaatggacgcaactattagccttcaaaaaaacggaaaaagtttttattgggcttctcattttcttggccgtgaaatgtctgaaaatgcagccaagttgtattcctactgtcgtatactagatgacttcgcagactcagaaattgagtatggcccagataggctcaggaatatatatcagaatttaaaaaataaaaaaaaatattctgacccgcttctggatgaaatccttatgttttttgaagatatgaacttacctttcagggctttgaaggacttaattgagggacttctgcaagatcaaaacgatgttctaattaaaaatgaggctgaattaattcaatatagctatcatgttgctggatcagtgggatggatgatgtgtccaatacttaaatgcagaaataaaaaagcatttgctttcgcagtagatctcggaattgctatgcaacttaccaatattgcacgagatgtacgggaagatgcttacatggggagacgatatttaccgtcagattgggtaaacgatatgtcgccaacagatattttagaggcttccaaaaaaccaaattcaaaaaatgctcaattagttaaaaaagcgattataaagctattatgtcttgcagatcagtattatcagagtggcgtaaaaggattagtttttttaccatggaaggcacatttagctattggtgttgcaggatgtgtttatcaaaaaataggaacccaattgatcgctaaaaaagttaattggtatgatggacgcacagtcacttccagttttgcaaaagttctatcaagctgcggtgtgtttcctcttttattttcgaggtttcttaaacctcctaaacataatgctaaactgcatcaataccttgcagaaaagctagatatgttatgagcattcaaaccaaaacggtaaaggcaaaaacggtggcaattgttggtggtggctgtgcaggctggtctcttgcagcaagagtcgaccaactatctgctaatacggtaactctctacttaaaagaggaaacgaacccttcccataattggggattctggcagatgccttggctttctgatgcgttgtcgaaaaaaagggcaatgtggaatagttggcaaatcataactgacgagggaatagtaacccatcatagccgtaaccacgcttattgcagtattacgagtgatgattggttcgattggtgccaaaaaagatttcattccgcagctaccacaacggagatcataaggttacctgtaactaacacagataaaaatatgctaatgacatcgaatcataaaaaaagatatgatttaatttatgattctcgtcctcctaaggcagataagaacattctacttcagcattttaaaggtttggaaatcaaaacatcaatcccggtttttaattctaaggctgccgttttaatggacttcagagtgtctcaagaaaacggtattcatttcatgtatgtgctaggttatgaccaatatacagctctagtagagtcaacgttttttagcacatcaccccatcgagaagaagtttatgacgaagcaataagagactatttaagtcgtatttataatgttaccagcttcgaaatcgtcaaaagtgaaaaaggtataatcccaatgggggatgtaagaccaaaagatcaaaatccttttttgtattctattggaagcaatgctggagctattagaccctcatctggctatgcatttagctttattcaaaagcaaattacacaatttttagatcgtcaaacaaaaccaacaaacccacataaaaaacatgacttaatgatggatctcatatttttagaggttcttaaatcccacccagaaaaagcacagagacttttctttaaattagcaagagcgttaaatggtgactcatttgctcgatttttaagtggtgaggccaatattaaagattattttttagttataaactcaatgcctaaatggctatttattaatcaagccgttagagttatcatagaaaagtggaaaaggatagctaaatgggcttgatgttaattgattggtgtgctttagcattggttgtgtttattggtttgccacatggtgccttagatgctgctatttctttttcaatgatttcttcagcaaagagaattgctagattagcaggaatactattaatttacctgttgttagcaaccgcattttttttaatttggtatcaattaccagcattttctcttcttatttttcttttgataagcataatccattttggaatggctgatttcaatgcatccccaagtaaacttaagtggcctcatattattgcacatggcggcgttgttactgtttggttgccgcttatccaaaaaaatgaagttacgaagctattttcaatattaacaaatggtccaactcccattttatgggacatactattgatattttttttatgttggagcataggagtatgtcttcatacctatgaaactttacgttctaaacattataatatcgcctttgaacttattggattaatttttctagcctggtatgcacccccactcgttacttttgccacatacttctgctttatccacagcagacgtcactttagttttgtttggaaacagttacagcatatgagttcaaaaaaaatgatgataggtagtgccattattttatcttgtacgagctggttgataggcggaggaatatattttttcctcaattcgaaaatgattgccagtgaagctgctttacaaactgtctttattggtcttgcagctttaacagttcctcacatgatacttatcgactttatatttagaccacactcttccagaattaaaatcaaaaattgagacagttttttatgattacccccaccacaaaagatttaatttcttctgctcgtaaagatattcatattgatttatctaaatcagagctttctcgcttcaatattgttcaccctttagacctaattacgctaccacataatgctcttccagaaatggattttgatgatgtagatacttcttgtgaatttctaaacaaagaattatcgtttccatttatgataacaggaatgacaggtggtactcctcgcgggaatagactgaatctcgcctttgcagaggtggctaaccaatgcggtattgcttttggtgtaggaagtcaaagaagtagcattgctaattgtaaaagccaaaaagagttgaggaaattagctccaaaaattcccattattggtaacattggaggcattcaacttgcgcagaaaaatggtttagaattagctagggctgctatagaagaccttgaggcagatgctcttgcaattcatttaaatccgctccaagagatcatccaaccagagggagaatctaattggaggggcgttctaaattcgattgaaaaagcagtaaaaacgttaccatgtcctattctcgtcaaggaagtgggtgcgggcataagtttacctgtggccaaaaaacttcataacgttggagtctaccacattgatgttgcttgtgcaggaggaacaagttgggcgaggattgaagctgaacgacttccaaacagccagcgtgagctctatgagccatttcttgattggggacacctaatcacagatattttgcccgaaatgcgacaaacccttcagcaggtgactataattggctctggcggtttacgtaatggacttgatttagctaaactattatatctcggctgtcatattggtggcggcgcatctcttttactaaaatcgttggaaacagaagaattagaagttaaacaagagcacctttttcaatcactgaaaacaataaaagaacaattatccatctccttatttcttacgggaagcaataaagcagatgatttgattagcttaaataagatgtcatag

SEQ ID NO: 71 - DNA, 1203 bp, Artificial Sequence
  Synthetic nucleic acid sequence cloned from Salmonella typhimurium
atgacacaattcgcttctcctgttctgcactcgttgctggatacagatgcttataagttgcatatgcagcaagccgtgtttcatcactattacgatgtgcatgtcgcggcggagtttcgttgccgaggtgacgatctgctgggtatttatgccgatgctattcgtgaacaggttcaggcgatgcagcacctgcgcctgcaggatgatgaatatcagtggctttctgccctgcctttctttaaggccgactatcttaactggttacgcgagttccgctttaacccggaacaagtcaccgtgtccaacgataatggcaagctggatattcgtttaagcggcccgtggcgtgaagtcatcctctgggaagttcctttgctggcggttatcagtgaaatggtacatcgctatcgctcaccgcaggccgacgttgcgcaagccctcgacacgctggaaagcaaattagtcgacttctcggcgttaaccgccggtcttgatatgtcgcgcttccatctgatggattttggcacccgtcgccgtttttctcgcgaagtacaagaaaccatcgttaagcgtctgcaacaggaatcctggtttgtgggcaccagcaactacgatctggcgcgtcggctttccctcacgccgatgggaacacaggcacacgaatggttccaggcacatcagcaaatcagcccggatctagccaacagccagcgagctgcacttgctgcctggctggaagagtatcccgaccaacttggcattgcattaaccgactgcatcactatggatgctttcctgcgtgatttcggtgtcgagttcgctagtcggtatcagggcctgcgtcatgactctggcgacccggttgaatggggtgaaaaagccattgcacattatgaaaagctgggaattgatccacagagtaaaacgctggttttctctgacaatctggatttacgcaaagcggttgagctataccgccacttctcttcccgcgtgcaattaagttttggtattgggactcgcctgacctgcgatatcccccaggtaaaacccctgaatattgtcattaagttggtagagtgtaacggtaaaccggtggcgaaactttctgacagccctggcaaaactatctgccatgataaagcgtttgttcgggcgctgcgcaaagcgttcgaccttccgcatattaaaaaagccagttaa

SEQ ID NO: 72 - DNA, 2511 bp, Artificial Sequence
  Synthetic nucleic acid sequence cloned from Ralstonia eutropha
atgaacgcgcctgtatgtaccggtcttgcctcagcaaaacccggcgtattgaatgtgctctggatacagtccggcggctgcggtggctgcagcatgtccctgctttgcgcggacactaccgatttcactggcatgctcaagagtgcgggaatccacatgctttggcatccctcgctatcgctggaaagcggcgtggaacagctgcaaatattggaagactgcttgcaaggccgcgtcgcactgcatgccttgtgtgtcgagggggcgatgctgcgcggtccgcatggtaccgggcgctttcatctcctcgccggcactggtgtgccgatgatcgaatgggtcagccgcctggcagcggtcgccgactacacgctggcggtcggcacctgtgccgcttatggcggcatcaccgcaggtggcgggaacccgacggacgcctgtggcctgcaatacgagggggaccagcccggaggactgctcggcctcaactaccgctcacgcgccggcctgccggtgatcaatgtcgccggttgcccgacgcatccgggttgggtgacggatgcactggcattgctgtcggctcgcctgctaaccgcgagcgacctggataccctgggccggcctcgattctatgctgatcagttggtgcatcacggttgcacgcgcaacgagtactatgaattcaaggctagtgcggaaaagccttcggacctgggttgcatgatggaaaacatgggatgcaagggtacccaggctcacgccgattgcaatacccgcctatggaacggagagggttcctgtacccgcggggggtatgcctgcatcagttgcactgaacccggcttcgaagagcccggccaccccttccaccaaacacccaaggttgccggcatcccgatcggtttgcccaccgatatgcccaaagcctggtttgtcgcgcttgcttcgttgtccaagtcagccacgcccaagcgagtgaaacttaatgccacggcagatcatccgctgatcgcgccagccattcgcaagacgcggctgaaataggaggcgagcatggaacgtttggtggtggggccgttcaaccgcgttgagggcgacctcgaggtgaacctcgaggtcgccagcggtcgggtatgctcagcacgtgtgaatgccacgatgtaccgcggattggaacagatcctgctacaccggcatccgcttgacgcgctggtctatgcgccgcgtgtgtgcggtatctgctcggtgtcccagtcggtcgcggccagccgcgcattggcggatcttgccggcgtgacggtacccgctaacggtatgctggcgatgaacttgatgctggcgaccgagaatctggccgaccatctgacgcacttctatctgttcttcatgccggatttcacgcgcgagatatatgctggccgcccctggcacaccgacgcgacggcacgtttttcgcccacgcatggtaagcaccacaggctggcgattgccgcaaggcagcgatggtttaccttgatgggcaccctgggcggcaaatggccgcacacagaatcggtgcagcccggtggttcgtcgcgcgccatcgacgccgctgagcgcgttcgcctgctgggccgggtgcgcgaattccggtgctttctcgagcagacactgtatgccgcaccgctcgaggacgtcgtcgcgctcgacagcgaagtcgcgttgtggcgctggcatgcgcaggcgccgcaggccggagacctgcgatgtttcctcacgatcgcgcaggatgcggcgctggaccagatggggcccggcccgggtacgtatctgtcatatggcgcctacccccagcccgaaggcggtttttgcttcgcacaaggtgtgtggcgtagcgcgcaagggcggctcgacgcgcttgatcttgccgcgatcagcgaagatgccacctccgcctggttagtcgaccaagggggggcgcgtcatcctgccaatggcctgaccgcgccagcacctgacaaggtgggcgcctatacctggaacaaggcccctcgattggccggcgccgtgctggagaccggtgcgatcgcccggcagttggccggcgcacagcccttggtgcgtgacgcggtggcgcgctgcggcgccaccgtctatacgcgcgtattggcgcgactggttgaactggcgcgagtggtgccgttgatggaagactggttacagtccctagaaatcggggccccatattgggcgtcggcccacctgccggatcagggcgctggcgttggccttacggaggcggcgcgcggcagcctggggcactgggtgtcggtgcgcgatgggcgaatcgacaattaccagatcgtcgcacccacgtcctggaacttctcgccgcgcgatatagccggccagcccggggccgtggagaaagcactcgagggtgcgcctgtgctgcaaggcgaaacaacgccggtcgccgttcagcacattgtccgctccttcgatccctgcatggtgtgcaccgtgcattga

SEQ ID NO: 73 - DNA, 4620 bp, Artificial Sequence
  Synthetic nucleic acid sequence cloned from Ralstonia eutropha
atggatagtcgtatcacgacaatactcgagcgctaccgctcagaccgtacacggctgatcgacatactttgggatgttcagcatgagtatgggcacattcccgatgcggtactgccgcaactgggggctgggttgaagctgtccccgctggacattcgcgaaacggcgtcgttctaccactttttccttgacaagccgtcgggcaagtatcggatttacttgtgcaattccgtgattgccaagatcaacggctatcaggcggtgcgtgaggcgctcgaacgcgagactgggattcgcttcggcgaaaccgacccgaatgggatgtttggcctgttcgacaccccctgtatcggactcagcgatcaggaaccggcgatgctgatcgataaggtggtattcacccgcctgcgacccggaaagatcacggacatcatcgcgcagttgaaacaaggacgatcgccggccgagatcgcgaacccggccggtttgcccagtcaggacatcgcctatgtcgatgccatggtcgagtccaatgtccgcaccaaggggccggtgttcttccgtggccggacggatttgagatctttgctcgaccaatgcctgctgctcaagcccgaacaagtgattgagaccatcgtcgactccaggctgcgcggacgtggcggcgcagggttctcgaccgggctgaagtggcggctgtgtcgggatgccgaaagcgagcagaagtatgtaatctgcaacgccgacgaaggtgagcccggcacgttcaaggatagggtcctcctgacacgcgctcccaagaaggttttcgtcggaatggttatcgccgcgtatgcgatcggctgccgcaagggtatcgtctatctgcggggggaatacttctacctcaaggattatctggagcgacagcttcaggaacttcgggaggacgggttgctggggcgcgctatcggtggccgggcgggctttgatttcgatatccgtattcagatgggggccggcgcttatatctgcggcgacgaatcggcgctcatcgagtcctgcgaggggaaacggggcacgccacgggtgaaacctccgttcccggtgcagcaagggtatctgggcaagcccaccagcgtcaacaacgttgagacctttgccgccgtgtcgcggatcatggaggaaggcgcggactggttccgggcgatgggaacgccagactcggccggcacccggctgctgagcgtggctggcgattgcagcaagcctggcatctacgaggtggaatggggggtcaccctcaacgaagtgctggcgatggtcggagcgcgggacgcgcgggccgtccagatcagcggtccttccggtgaatgcgtgtcggtggcaaaggacggtgagcgcaagctcgcgtacgaagatctttcgtgcaatggcgccttcaccattttcaactgcaagcgcgacctgctggaaatcgtgcgtgaccacatgcagttcttcgtcgaagagtcctgcggcatttgtgtgccatgtcgcgccggcaacgttgatctgcaccggaaggtcgaatgggtcatcgcgggcaaggcctgccagaaggatctggacgatatggtcagttggggagcgctggtgcggaggaccagtcgatgtggccttggggccacatcgcccaagcccatcctgacgacgctggagaaattccccgagatctatcagaacaagctggtgaggcacgagggcccgctgctgccatcgttcgatctcgataccgccttgggcgggtatgagaaggcgctgaaggatctggaagaggtgacaagatgagcattcaaattacgatcgacggcaagacgctcacgaccgaggaaggacgaacgctggtggatgttgccgcagagaacggcgtttacatcccgacgctgtgctacctcaaggacaagccctgcctcggcacctgccgggtgtgttcggtcaaggtgaatggcaatgtcgccgcggcatgtacggtgcgggtctcgaagggcctgaatgtcgaggtcaacgaccccgaattggtcgacatgcgcaaggcgctggtcgaattcctgttcgcggaaggcaaccacaactgcccgagttgcgagaagagcggccgttgccagttgcaggcggtcggctacgaggtggacatgatggtctcgcgctttccgtaccggttcccggtccgcgtggtggaccacgcgtccgaaaagatctggctcgagcgggatcggtgcatcttctgtcagcgctgtgtcgagttcatccgcgacaaggcaagcggccggaagatcttcagcatcagccatcggggtcccgagtcgcgcatcgagatcgatgccgaactggcgaacgccatgccgccggagcaagtcaaagaggcggttgcgatctgcccggtgggcaccattctcgagaaacgggtcggttatgacgatcccatcggacgacgcaagtacgaaatccagtcggtgcgcgcacgcgcgctggaaggagaagacaaatgagagccccccacaaagacgagattgcgtcgcacgaattgcctgcgacgccgatggatccggcgctggccgcgaaccgtgaaggcaagatcaaggtggccacgatcggtctgtgcggctgctgggggtgtaccttgtcctttctcgacatggacgagcggctcctgccgctgttggagaaagtcaccctcctccgctcgtcgctgaccgatatcaaacgaattccggagcgctgtgcgatcggcttcgtggaaggtggcgtctcgagcgaggagaacatcgaaacgctggagcactttcgcgagaactgcgacatcctgatttcggtcggggcgtgcgcggtgtggggcggtgtgccggcgatgcgcaacgtcttcgagctgaaagattgtctggcagaggcttatgtcaactcggcgactgccgtcccgggcgccaaggccgtcgttccattccatcccgatatcccgaggatcaccaccaaggtctacccttgtcatgaggtggtcaagatggattatttcattccgggttgtcccccggatggagatgccatcttcaaggtgctggacgatctggtgaatggacggccattcgatttgccgagctcgatcaatcgctacgattgattaacggagggtttgttatgagcagaaaactggttatcgacccggtgacccgcatcgagggtcacggcaaggtggtggtgcacctggatgacgacaacaaggtcgtcgacgcaaagctgcacgtcgtggagttccggggctttgaaaagttcgttcagggccatcccttctgggaggcgccgatgttcctgcagcgcatctgcggcatctgtttcgtcagtcaccatctgtgcggggccaaggcgctggatgacatggtcggcgtcggcttgaagtccggcatccatgtcaccccgacggcggagaaaatgcgccggctcggccactacgcgcagatgctccagtcccatacgaccgcctatttctacctgatcgtgccggagatgctgtttggcatggacgcgccgcccgcacagcgcaacgtgctcggcctgatcgaggccaatcccgacttggtgaagcgcgtcgtgatgttgcgcaaatggggacaggaagtcatcaaggcggtgttcggcaagaagatgcacggcatcaattcggtgccgggaggcgtcaacaacaacctgagcatcgccgagcgcgaccgtttcctgaacggggaggagggccttctgtcggtggatcaggtcatcgattacgcgcaggatggcctgcgcctgttctacgacttccatcagaaacaccgggcgcaggtcgatagtttcgcggacgtgcccgcgctcagcatgtgcctggtcggcgacgacgacaacgtggactactaccacggcaggctgaggatcatcgacgacgacaagcacatcgtccgtgaattcgactatcacgactatctggatcatttctccgaagcggtggaggaatggagctacatgaagttcccctacctcaaggagcttgggagagagcagggatcggtgcgcgtggggccgcttggccgcatgaacgtgacgaagtcgctcccgacaccgctcgcgcaggaggcgctggaacggttccacgcctacacgaaggggcggacgaacaacatgacgctgcatactaactgggcacgggccatcgagatcctccacgccgcggaggtggtcaaagaactgctgcatgacccggacctgcagaaggatcagctggtgttgacaccgccccccaatgcgtggacaggtgaaggcgtcggcgtggtcgaagcgccacgcggtaccttgcttcaccattatcgtgccgatgagcgcggcaatatcacgttcgccaacctggtcgtcgccaccacccagaacaaccaggtcatgaatcgcacggtgcgcagcgtcgccgaggactacctgggcggacatggcgaaatcaccgagggcatgatgaatgccatcgaggtgggtattcgcgcctacgatccatgcctgagctgcgcgacacacgccctggggcagatgccgctggtggtctcggtctttgacgcggcggggcgcctgatcgatgaacgcgcccgctga

SEQ ID NO: 74 - DNA, 4090 bp, Artificial Sequence
  Synthetic nucleic acid sequence cloned from Ralstonia eutropha
atggtcgaaacattttatgaagtcatgcgcaggcagggcatttcgcgacgaagtttcctgaagtactgttccctgacagccacatccttaggactgggaccttcctttctgccgcagatcgcgcacgcgatggaaaccaagccgcgtacaccagtactttggctgcacggtctcgaatgtacctgttgctcggaatcgttcattcgctcggcccatccgctggcaaaggacgtcgtgctatcgatgatctcactggactatgacgacacactgatggcggctgccggccaccaggccgaggccatcctcgaggagatcatgacgaagtacaagggcaactatattctggcggtggaggggaatccgccactcaatcaggatggcatgagctgcatcatcggtgggcggccattcattgagcagctcaaatacgtggccaaggatgccaaggccattatctcctggggttcctgcgcatcctggggatgcgtgcaggcagccaaacctaatcccactcaggccacaccggttcacaaggtgatcaccgacaagccgattatcaaggtcccggggtgccctccgattgccgaagtgatgacgggtgtcattacctacatgctcaccttcgatcgtattcccgaactggatcgacagggtcggccgaagatgttctatagccagcgcatccacgacaaatgctaccggcgtccacacttcgatgccggccagttcgtcgaggaatgggacgacgaatcagcccgcaaaggcttctgcttatacaagatgggctgtaaaggcccgaccacgtacaacgcctgctccaccacgcgctggaacgaggggacgagtttccccattcagtcgggccacggttgcattggttgctccgaggatggcttttgggacaaaggctcattctacgatcgtctgaccggcatcagccagttcggcgttgaggccaacgccgacaagattggcggaacggcctccgtcgtggtgggggcggccgtgacggcgcatgccgcagcgtctgcgatcaagcgtgcgtcgaagaagaacgaaaccagcggcagtgaacactaagccgccggggaaacgactgaatcaggaagatcgaaataatgtcagcttacgcaacccaaggcttcaatcttgacgaccgcggccgtcgcattgtcgtcgatcccgtcacccgcatcgagggtcatatgcgctgcgaggtgaatgtcgatgccaacaatgtcattcgcaacgctgtttccactggtaccatgtggcgcggactggaagtgattctcaagggccgcgatccgcgcgacgcctgggcgttcgtagaacgcatctgcggtgtttgtaccggttgtcacgcgcttgcgtcggtgcgtgccgtggaaaacgcgctcgacatcagaattccaaagaacgcccatctgatccgagagatcatggccaagacgttgcaggtgcatgaccatgcggtgcatttctatcacctgcatgcgctggattgggtggatgtcatgtcagccctgaaagccgacccgaagaggacttccgagttgcagcagttagtttcgcctgcgcatccgctgtcctcggcaggctatttccgcgatattcaaaatcgactcaagcgctttgtcgagagtggtcagcttggccctttcatgaatgggtactggggatccaaggcttatgtgctgccgccggaggccaatctgatggcggtcacgcattatttggaagcgctggacctacagaaggagtgggtgaaaatccacaccatcttcggcggcaagaatccgcacccgaactacttggtcggtggcgtgccgtgcgcgatcaatctcgatggtatcggggctgccagcgcgccggtaaatatggagcgcttgagcttcgttaaggcgcgcatcgacgagatcatcgaattcaataagaatgtatacgtgccagacgtgctcgccatcggcacactgtataaacaggccgggtggctgtacggcggcgggctggcagccaccaacgtgcttgactacggcgagtacccgaacgttgcctacaacaagagcactgaccaactgcccggcggcgcgatcctcaacggcaactgggacgaagtatttccagtggatccgcgcgactcccaacaggtgcaggaattcgtgtcgcacagctggtacaagtatgccgacgagagcgtaggtctgcatccctgggacggcgtgactgagcccaattacgtgctcggtgcaaacactaagggtacacgcacgcgcatcgagcaaatcgacgagagcgcgaagtactcgtggattaaatcgccgcgctggcgcggccacgcgatggaggtagggccgctgtcgcgctacatccttgcctatgcccatgcgcggagcggcaacaagtacgctgagcgtcccaaggagcagcttgagtactccgcgcagatgatcaacagtgcgataccaaaggcattgggattgccagaaacacaatacacgctcaagcagttgttgcccagcacgatcggtcgtacgctggcgcgcgcactcgagagccaatattgcggagaaatgatgcatagcgactggcatgatctggtcgccaacatccgggcgggcgatacggcaaccgccaacgttgacaagtgggatcctgccacctggccgctgcaagccaagggcgttgggaccgtcgctgcgccgcgcggcgctctcggacactggattcgtatcaaggacggccggatcgagaactatcagtgcgtagtgcctaccacgtggaatggcagtccgcgtgattacaaggggcagatcggcgcatttgaggcttcgctgatgaacaccccgatggtcaacccggagcagccggtggaaatcttgcgcacgctgcattcgttcgatccctgtctggcgtgttcgactcacgtcatgagcgcggaaggccaggaactcactacagtcaaggtgcgataaaaggtgccggaccggcgtctggccagcgagcggatatcggttcggcaacggcacaaggagatagcatgagcacaaaaatgcaggcggatcgcattgcagatgcgaccgggaccgacgaaggagcggtagccagcgggaagtcaatcaaggccacttatgtttatgaggcgccagtgaggctgtggcactgggtcaatgcgctggcgatcgtagtgctggcagtgaccggattttttatcggctcgccgcccgcgaccaggccgggggaggccagcgcaaactttctgatgggctatattcgctttgcccactttgtcgcagcttacatattcgcgatcggcatgctgggccgcatctactgggcgacggcagggaatcatcattcccgcgaactcttctccgtgccggtgttcactcgggcgtactggcaggaggtgatttcgatgctgcgttggtacgccttcctatctgcgcgtccaagccggtatgtcggtcacaatccgctggcccgtttcgcgatgttcttcatcttcttcctgagttcggtgttcatgatcctcacgggcttcgcgatgtacggcgaaggcgcacagatgggctcgtggcaggagcgcatgttcggctgggtcattcctttgctcggtcaatctcaggatgtgcatacctggcatcatttgggtatgtggttcattgtggtgtttgtgatcgtccatgtctatgcagcgattcgcgaggacatcatgggccgccagagcgtagtgagcacgatggtctcgggctatcggacctttaaggactgatgcgcaatgcaaccgaagcgatcgcaggctttcgtcgataccatgcacccgctaccggaggtattgccctcggtatggtggcatcccggcgtgtcacggcgcgtgtcggttcattcgggctgttttctgagtagtcattacctcaagccgctcggcctgtcgcagccccacgcccgctgtctgctcaggattgcgaaacggcgcttgcatgaaatcatcaattggaacagccgtcaggcttggaaaggcgcctgttcctcctttcgtgtctcggtcgctcgccgcacggtacacggttccctttttcgctga

SEQ ID NO: 75 - DNA, 6950 bp, Artificial Sequence; identical to SEQ ID NO: 66
  Synthetic nucleic acid sequence cloned from Ralstonia eutropha

SEQ ID NO: 76 - DNA, 3779 bp, Artificial Sequence
  Synthetic nucleic acid sequence cloned from Ralstonia eutropha
atgcatgagatgtcgctggccgtgggtgtgctgcagatcgtcgaggacgtggcgcagcgcgacggtttttcccgcgtaacggcagtacggctggagatcggccggctgtccagtatcgagccggaagcgctgcgcttctgcttcgaggaggtagtgcgcggcagtgtggctgacggcgcgcggctggagatcgtggacacccccggtgccggctggtgcctgcactgcagcgagacggtggcgatcggagcgctgtacgatccctgcccgcagtgcggcggctaccaggtgcagcccactggcggcacggaaatgcgcgtcatggatctggaagtggcgtgacggaaagcggaagtcacgcggaatagaagacgaggagacaggatcatgtgcaccaactgtggctgcgccgccggcgaaacccgcatcgaaggccaggagctggagcatgtcgacgagcatgcgcatgccgacggcaccgtccatggccacgccccccatcatcacggcgagcacgatcacgaccacggccctgcctaccgtgccgtgcgcggtactgaccatctgcattacggtcacggacccgctggtgcccatgccccaggcatgagccaggcgcgcatggtgaagatcgagcaggacatcctgggcaagaacaacgcctacgcggcgcagaaccgccgctggttcgacgagcacggcgtgttcgcgctgaacttcgtctcgagccccggctccgggaagaccaccctgctggtgcgcaccatcgaggcgctgaagtccacctccaggctggccgtcatcgaaggcgaccagcagacttccttcgatgccgagcgcatccgcgccaccggcgtgcaggcgctgcagatcaacaccggcaagggctgccacctggatgcccacatggtgggccacgcgctggagaaactgcggccggaggacgagagcgtgctgctgatcgagaacgtgggcaacctggtctgcccttcggccttcgacctgggcgaagcccacaaggtggtgatcctttcggtcaccgaaggggaggacaagcccctgaaatatcctgacatgttccgcgccgccagcctgatgctgctcaacaagtgcgacctgctgccgcacctgtccttcgacgtggagagggccatcgagtatgcgaagcgggtgaatccggacctgcacgtgatccggacctcgtccgccaccggtgaaggcttcgacgcatggctgacgtggattgccgatggcctggccggccaggccgccaggcgcagccagagcatggagctactgcgcagccgcatcgccgggctggaagcgcaactggcggcgctcaaggtgtagcggccatgctgatgccgcgccgtcctcgcaatccgcgcaccgtccgcatccgcatccgcgtgcgcggcgtcgtgcagggcgtgggcttccggcccttcgtctatcgcctggcgcgggagctgggcctcgccggctgggtgcgcaacgacggcgccggcgtggacatcgaggcccaagggagcgccgcggccctcgtcgagctgcgcgagcgcctgcgccgcgacgcgccgcccctggcgcgggtggatgagatcggtgaggaacgctgtgcggcacaggttgacgccgacggctttgccatcctcgagagcagccgttccgatgccgccgtgcacaccgccatcggccacgacacggccgtatgccccgactgcctggcggagctgttcgaccccgcgaaccggcgctaccgctacgccttcatcaactgcacccagtgcggcccgcgctacaccctgacgtgggcgctgccctacgaccgcgccaccaccagcatggcgccgttcccccagtgccggccctgtctcgacgagtataacgcacccgagcatcgccgcttccacgccgagcccaacgcctgtccggactgtggccccagcctggcgctgctcaatgcccagggcatgccggtcgaggacgttgatccgatcgccgagacggtggcgcgcctgcagcggggcgagatcgtcgccatcaagggccttgggggcttccatctcgcctgtgacgcgcacaatgccgacgcggtggcgcggctgcgcagccgcaagcagcgcgaggagaagcccttcgccgtcatggtcgcgaacctggcgacggcggcgcagtggggcgacatcggcagcggtgaagcggccctgctcacggcgtcggagcggcccatcgttctgctgcgcaagcgcagcggcgtcgatgggcggttcgcgggcgtggcgccgggactggtctggctgggcgtcatgctgccgtacacgccgctccagtatctgctgttccacgaggccgctggccgccccgagggcctcggctggctcgcccagccgcagtcgctggtgctggtgatgaccagcgccaaccccggcggcgagcccttggtcaccggcaacgacgaggcggcgcagcggctcactggcatcgccgacgctttcctgctccacgatcgcgagatcctggtgcgttgtgacgattcggtggtgaggggcgacggcgaaccagcgcctcacgtccagttcattcgccgcgcgcggggttacacgccgcgggcgatcaagctggcgcgcagcggcccctcggtgctggcgctgggcggctccttcaagaacacggtgtgcctgacgcgcggcgacgaggccttcgtctctcagcatgtgggcgatctcggcaacgctgccacctgcgaggccctgatcgaggcggtggcccatctgcagcgggtgctggagatccggccgcagctcgtcgcccacgatctgcatccggacttcttcagcacacgccacgcagcggagctggcggcgcaatggggggtgcctgccgtcgccgtgcagcaccatcatgcccatattgcggcggtgctggccgagcacggctcggacgaacccgccatcggcctggcgctggacggcgtcggcctgggcgacgacgggcaggcgtggggtggcgaactgctgctggtggacggcggcgcctgtaagcgcctcggtcacttgcgcgagctgccgctacctggcggcgatcgcgccgcgcgggagccctggcgcatggccgctgcggcgctgcatgcgatgggccgcggcgaggagatcgaggggcgcttcccgcggcagccgggggcgccgatggtgaaccgcatgctggcccagcggcttaacgcgcccctctcctccagcatgggccgctggttcgacgccgccgccggcctgctcggcacacgggaaaccatggcctacgaggggcaggcggcgatgctgctggaagggttggccgagagctggggcgagcaaccgtcgccgggccgccccaagacggttgctcactccctcgggggggtgccgcgcagcggcgggggtacatacaaggcgctggcgctgcccgatgcctggcgcatcgatgcgggcaacaccctggatctgctgccgctgctcgaggcgctgtccgccgaaacgaatgccgcgcgcggcgcggcacagttccacgccaccctggtggcggcgctggaagcctggaccgtcgcgaccgtccaggtcaccggcgtgcgcacggtcgtcttcggcggcggctgcttcctcaaccacatcctcgcccgcaacctgtgccggcgcctggcggcgcggggactgaccgtgctgacagcgcgccagctgccgcccaatgacggaggcattgccctcggtcaggtctgggtggcgctgcagcgggcgcccaactga

SEQ ID NO: 77 - DNA, 1749 bp, Artificial Sequence
  Synthetic nucleic acid sequence cloned from Clostridium acetobutylicum
atgaaaacaataatcttaaatggcaatgaagtgcatacagataaagatattactatccttgagctagcaagagaaaataatgtagatatcccaacactctgctttttaaaggattgtggcaattttggaaaatgcggagtctgtatggtagaggtagaaggcaagggctttagagctgcttgtgttgccaaagttgaagatggaatggtaataaacacagaatccgatgaagtaaaagaacgaatcaaaaaaagagtttcaatgcttcttgataagcatgaatttaaatgtggacaatgttctagaagagaaaattgtgaattccttaaacttgtaataaagacaaaagcaaaagcttcaaaaccatttttaccagaagataaggatgctctagttgataatagaagtaaggctattgtaattgacagatcaaaatgtgtactatgcggtagatgcgtagctgcatgtaaacagcacacaagcacttgctcaattcaatttattaaaaaagatggacaaagggctgttggaactgttgatgatgtttgtcttgatgactcaacatgcttattatgcggtcagtgtgtaatcgcttgtcctgttgctgctttaaaagaaaaatcccatatagaaaaagttcaagaagctcttaatgaccctaaaaaacatgtcattgttgcaatggctccatcagtaagaactgctatgggcgaattattcaaaatgggatatggaaaagatgtaacaggaaaactatatactgcacttagaatgttaggctttgataaagtatttgatataaactttggtgcagatatgactataatggaagaagctactgaacttttaggcagagttaaaaataatggcccattccctatgtttacatcttgctgtcctgcatgggtaagattagctcaaaattatcatcctgaattattagataatctttcatcagcaaaatcaccacaacaaatatttggtactgcatcaaaaacttactatccttcaatttcaggaatagctccagaagatgtttatacagttactatcatgccttgtaatgataaaaaatatgaagcagatattcctttcatggaaactaacagcttaagagatattgatgcatccttaactacaagagagcttgcaaaaatgattaaagatgcaaaaattaaatttgcagatcttgaagatggtgaagttgatcctgctatgggtacttacagtggtgctggagctatctttggtgcaaccggtggcgttatggaagctgcaataagatcagctaaagactttgctgaaaataaagaacttgaaaatgttgattacactgaagtaagaggctttaaaggcataaaagaagcggaagttgaaattgctggaaataaactaaacgttgctgttataaatggtgcttctaacttcttcgagtttatgaaatctggaaaaatgaacgaaaaacaatatcactttatagaagtaatggcttgccctggtggatgtataaatggtggaggtcaacctcacgtaaatgctcttgatagagaaaatgttgattacagaaaactaagagcatcagtattatacaaccaagataaaaatgttctttcaaagagaaagtcacatgataatccagctattattaaaatgtatgatagctactttggaaaaccaggtgaaggacttgctcacaaattactacacgtaaaatacacaaaagataaaaatgtttcaaaacatgaataa

SEQ ID NO: 78 - DNA, 1053 bp, Artificial Sequence
  Synthetic nucleic acid sequence cloned from Clostridium acetobutylicum
atggataatataataaagttaattaataaagcagaggtaacacatgatttaacaaaggatgaattagtaactttattgaaggatgatactcataatgaagagatatataaagcagccgatagagtacgtgaaaaatatgttggagaagaagtacatttaagaggtcttattgaattctctaatatttgcaaaagaaattgtatgtattgtggtttaagacgtgataataaaaatataaaaagatatagactagaacctgatgaaataatacatcttgctaagagtgcaaagaattatgggtatcaaacagttgtacttcaatctggtgaagatgactattatactgttgaaaaaatgaaatatatagtaagcgaaattaaaaagctaaacatggctattactttaagcattggagaaaaaacctttgaagagtatgaagagtacagaaaatctggagctgatagatatttaataagaatagaaacaacagataaagaactgtatgaaaagttagaccctaaaatgagccatgaaaataggataaactgccttaaaaatttaagaaagcttggatatgaagttggttctggatgtcttgtaggtcttccaaatcaaactatagaatccttggcagatgatatattattttttaaggaaatagatgctgatatgataggggtaggaccttttatacctaacgaggatactcctcttggagaggaaaagggtggagagtttttcatgagtgtaaaggtaactgcattgattagacttcttctgccagatataaatataccggcaactacggcaatggaatccctttatccaaatggaagatcaatagctcttacaagcggtgcaaatgttgttatgcctaatgttacagaaggtgagtatagaaagctctatgccctttacccaggaaaaatatgcgtaaacgacactcctgggcactgcagacaatgtatatcactaaaaataaataagataaatagaaaagtgtctgctacaaagggctttagaaaaaaaagctacaaagaatctattggttaa

SEQ ID NO: 79 - DNA, 1236 bp, Artificial Sequence
  Synthetic nucleic acid sequence cloned from Clostridium acetobutylicum
atgaatgaacttaactcaacacccaaaggtgaaagacttcacattgcactttttggaaaaactaatgttggaaaatccagtgtaataaatgctcttacttctcaagaaatagctcttgtttcaaatgtaaaaggtacaacaactgatcctgtttacaaagcaatggaactgctccctcttggaccagttatgcttatagatactgctggtcttgatgacataagtgatttaggtgagcttagacgtggaaaaactctcgaggttttatcaaaaactgatgtagcaattttagtttttgatgttgaaagtggaataacagaatatgataaaaatatttactcattactgcttgaaaagaaaattccactaataggagttttaaataaaatagataaaaaggactataagctagaggattatacttctcaatttaaaatacctatagtaccaatttcagctctcaataataaaggcataaataatttaaaagatgaattaatacgcttagcaccagaaaatgatgataaatttaagattgttggtgatttactttctcctggtgacatagcagttttagttactccaatagacaaagctgctccaaagggaagattaatacttcctcagcagcaaactataagagatatacttgaaagtgacgctattgctatggtgacaaaggaatttgaacttagagaaaccttagacagccttagaaaaaaacctaaaatagtaataacagattctcaagtatttttaaaggtagcagcagacacacctaaggacatattgatgacctcattttctatacttatggcaagacataaaggagatttaatagaacttgctcgtggtgcaagggcaattgaagatttaaaagatggtgataaaattttaatagcagaagcctgcacccaccaccgtcaatctgatgatataggtaaagtaaaaataccaagatggcttagacaaaaaactggtaaaaaactagaatttgatttttcaagtggcttttcatttcctccaaatatagaggattatgcacttatagttcattgtgctggctgcatgctaaacagacgttcaatgcttcacagaatagaaagttcagtaaaaaaacaaattcctatagtaaattacggagttcttattgcctacgttcaaggaattcttccaagggcattaaagccttttccatatgctgatagaatatttaatcaatcgagtaggaactaa

SEQ ID NO: 80 - DNA, 1419 bp, Artificial Sequence
  Synthetic nucleic acid sequence cloned from Clostridium acetobutylicum
atgtataatgttaaatctaaagttgcaactgaatttatcagtgatgaggaaattattgattcccttgaatacgcaaaacaaaataaaagtaaccgcgaacttatagacagcataattgaaaaagcaaaagaatgtaaggggttaacacacagagacgcagctgtattactagagtgtgacttagaagatgaaaatgaaaaaatgttcaagcttgcaagagaaatcaaacaaaaattttacggtaatcgtatagtaatgtttgctcctctttatctttcaaactactgtgtaaacggatgtgtatattgtccataccatcataaaaacaaacatatagcaagaaaaaagctgtcacaggaagatgttaaaagagaaacaatagctcttcaagatatgggacataaacgtttggctttagaggctggagaagaccctgtaaacaatcctattgaatatattcttgactgtatcaaaaccatatacagcataaaacataaaaatggagcaattagacgtgtaaatgtaaatattgcagctactactgtagaaaactacaagaaattaaaggatgctggtattggaacatatatacttttccaagaaacctataacaaaaaaagttacgaggaacttcatcctacaggtccaaaacatgattatgcctatcatacagaagcaatggatcgtgctatggaaggtggtattgatgatgtaggtattggggttttgtttggactaaatatgtacaaatatgactttgttggacttctaatgcatgctgaacacttggaagctgctatgggtgtaggccctcatactataagcgttcctcgtatacgtcctgcagatgacattgatcctgaaaacttctcaaatgcaatatcggacgagatttttgaaaaaattgtagccattattcgtattgcagttccatacacaggcatgatcgtctctactcgtgagtctaaaaaaactcgtgagcgcgtacttgaactcggtatatctcaaataagtggtggttctagcacaagtgttggtggttacgttgaatctgaacctgaagaagataactcttcacaattcgaagtcaatgataaccgtactcttgatgagatagttaattggcttttagaaatgaattatattccaagcttttgtaccgcttgttatcgtgaaggaagaactggcgaccgtttcatgagccttgttaaatcaggacaaatagcaaattgttgccagccaaacgccttaatgactcttaaagagtacttggaggattatgcttcttctaatactcaaaagaatggtgaagcacttatagcttctgaagttgaaaaaatacctaacgaaaaggttaaatcaatagtaaaaaaacaccttaccgaattaaaagagggacaaagagattttagattctaa

SEQ ID NO: 81 - DNA, 5474 bp, Artificial Sequence
  Synthetic nucleic acid sequence cloned from Clostridium acetobutylicum
agcccatggtgaaaacaataatcttaaatggcaatgaagtgcatacagataaagatattactatccttgagctagcaagagaaaataatgtagatatcccaacactctgctttttaaaggattgtggcaattttggaaaatgcggagtctgtatggtagaggtagaaggcaagggctttagagctgcttgtgttgccaaagttgaagatggaatggtaataaacacagaatccgatgaagtaaaagaacgaatcaaaaaaagagtttcaatgcttcttgataagcatgaatttaaatgtggacaatgttctagaagagaaaattgtgaattccttaaacttgtaataaagacaaaagcaaaagcttcaaaaccatttttaccagaagataaggatgctctagttgataatagaagtaaggctattgtaattgacagatcaaaatgtgtactatgcggtagatgcgtagctgcatgtaaacagcacacaagcacttgctcaattcaatttattaaaaaagatggacaaagggctgttggaactgttgatgatgtttgtcttgatgactcaacatgcttattatgcggtcagtgtgtaatcgcttgtcctgttgctgctttaaaagaaaaatcccatatagaaaaagttcaagaagctcttaatgaccctaaaaaacatgtcattgttgcaatggctccatcagtaagaactgctatgggcgaattattcaaaatgggatatggaaaagatgtaacaggaaaactatatactgcacttagaatgttaggctttgataaagtatttgatataaactttggtgcagatatgactataatggaagaagctactgaacttttaggcagagttaaaaataatggcccattccctatgtttacatcttgctgtcctgcatgggtaagattagctcaaaattatcatcctgaattattagataatctttcatcagcaaaatcaccacaacaaatatttggtactgcatcaaaaacttactatccttcaatttcaggaatagctccagaagatgtttatacagttactatcatgccttgtaatgataaaaaatatgaagcagatattcctttcatggaaactaacagcttaagagatattgatgcatccttaactacaagagagcttgcaaaaatgattaaagatgcaaaaattaaatttgcagatcttgaagatggtgaagttgatcctgctatgggtacttacagtggtgctggagctatctttggtgcaaccggtggcgttatggaagctgcaataagatcagctaaagactttgctgaaaataaagaacttgaaaatgttgattacactgaagtaagaggctttaaaggcataaaagaagcggaagttgaaattgctggaaataaactaaacgttgctgttataaatggtgcttctaacttcttcgagtttatgaaatctggaaaaatgaacgaaaaacaatatcactttatagaagtaatggcttgccctggtggatgtataaatggtggaggtcaacctcacgtaaatgctcttgatagagaaaatgttgattacagaaaactaagagcatcagtattatacaaccaagataaaaatgttctttcaaagagaaagtcacatgataatccagctattattaaaatgtatgatagctactttggaaaaccaggtgaaggacttgctcacaaattactacacgtaaaatacacaaaagataaaaatgtttcaaaacatgaataaatggataatataataaagttaattaataaagcagaggtaacacatgatttaacaaaggatgaattagtaactttattgaaggatgatactcataatgaagagatatataaagcagccgatagagtacgtgaaaaatatgttggagaagaagtacatttaagaggtcttattgaattctctaatatttgcaaaagaaattgtatgtattgtggtttaagacgtgataataaaaatataaaaagatatagactagaacctgatgaaataatacatcttgctaagagtgcaaagaattatgggtatcaaacagttgtacttcaatctggtgaagatgactattatactgttgaaaaaatgaaatatatagtaagcgaaattaaaaagctaaacatggctattactttaagcattggagaaaaaacctttgaagagtatgaagagtacagaaaatctggagctgatagatatttaataagaatagaaacaacagataaagaactgtatgaaaagttagaccctaaaatgagccatgaaaataggataaactgccttaaaaatttaagaaagcttggatatgaagttggttctggatgtcttgtaggtcttccaaatcaaactatagaatccttggcagatgatatattattttttaaggaaatagatgctgatatgataggggtaggaccttttatacctaacgaggatactcctcttggagaggaaaagggtggagagtttttcatgagtgtaaaggtaactgcattgattagacttcttctgccagatataaatataccggcaactacggcaatggaatccctttatccaaatggaagatcaatagctcttacaagcggtgcaaatgttgttatgcctaatgttacagaaggtgagtatagaaagctctatgccctttacccaggaaaaatatgcgtaaacgacactcctgggcactgcagacaatgtatatcactaaaaataaataagataaatagaaaagtgtctgctacaaagggctttagaaaaaaaagctacaaagaatctattggttaaatgaatgaacttaactcaacacccaaaggtgaaagacttcacattgcactttttggaaaaactaatgttggaaaatccagtgtaataaatgctcttacttctcaagaaatagctcttgtttcaaatgtaaaaggtacaacaactgatcctgtttacaaagcaatggaactgctccctcttggaccagttatgcttatagatactgctggtcttgatgacataagtgatttaggtgagcttagacgtggaaaaactctcgaggttttatcaaaaactgatgtagcaattttagtttttgatgttgaaagtggaataacagaatatgataaaaatatttactcattactgcttgaaaagaaaattccactaataggagttttaaataaaatagataaaaaggactataagctagaggattatacttctcaatttaaaatacctatagtaccaatttcagctctcaataataaaggcataaataatttaaaagatgaattaatacgcttagcaccagaaaatgatgataaatttaagattgttggtgatttactttctcctggtgacatagcagttttagttactccaatagacaaagctgctccaaagggaagattaatacttcctcagcagcaaactataagagatatacttgaaagtgacgctattgctatggtgacaaaggaatttgaacttagagaaaccttagacagccttagaaaaaaacctaaaatagtaataacagattctcaagtatttttaaaggtagcagcagacacacctaaggacatattgatgacctcattttctatacttatggcaagacataaaggagatttaatagaacttgctcgtggtgcaagggcaattgaagatttaaaagatggtgataaaattttaatagcagaagcctgcacccaccaccgtcaatctgatgatataggtaaagtaaaaataccaagatggcttagacaaaaaactggtaaaaaactagaatttgatttttcaagtggcttttcatttcctccaaatatagaggattatgcacttatagttcattgtgctggctgcatgctaaacagacgttcaatgcttcacagaatagaaagttcagtaaaaaaacaaattcctatagtaaattacggagttcttattgcctacgttcaaggaattcttccaagggcattaaagccttttccatatgctgatagaatatttaatcaatcgagtaggaactaaatgtataatgttaaatctaaagttgcaactgaatttatcagtgatgaggaaattattgattcccttgaatacgcaaaacaaaataaaagtaaccgcgaacttatagacagcataattgaaaaagcaaaagaatgtaaggggttaacacacagagacgcagctgtattactagagtgtgacttagaagatgaaaatgaaaaaatgttcaagcttgcaagagaaatcaaacaaaaattttacggtaatcgtatagtaatgtttgctcctctttatctttcaaactactgtgtaaacggatgtgtatattgtccataccatcataaaaacaaacatatagcaagaaaaaagctgtcacaggaagatgttaaaagagaaacaatagctcttcaagatatgggacataaacgtttggctttagaggctggagaagaccctgtaaacaatcctattgaatatattcttgactgtatcaaaaccatatacagcataaaacataaaaatggagcaattagacgtgtaaatgtaaatattgcagctactactgtagaaaactacaagaaattaaaggatgctggtattggaacatatatacttttccaagaaacctataacaaaaaaagttacgaggaacttcatcctacaggtccaaaacatgattatgcctatcatacagaagcaatggatcgtgctatggaaggtggtattgatgatgtaggtattggggttttgtttggactaaatatgtacaaatatgactttgttggacttctaatgcatgctgaacacttggaagctgctatgggtgtaggccctcatactataagcgttcctcgtatacgtcctgcagatgacattgatcctgaaaacttctcaaatgcaatatcggacgagatttttgaaaaaattgtagccattattcgtattgcagttccatacacaggcatgatcgtctctactcgtgagtctaaaaaaactcgtgagcgcgtacttgaactcggtatatctcaaataagtggtggttctagcacaagtgttggtggttacgttgaatctgaacctgaagaagataactcttcacaattcgaagtcaatgataaccgtactcttgatgagatagttaattggcttttagaaatgaattatattccaagcttttgtaccgcttgttatcgtgaaggaagaactggcgaccgtttcatgagccttgttaaatcaggacaaatagcaaattgttgccagccaaacgccttaatgactcttaaagagtacttggaggattatgcttcttctaatactcaaaagaatggtgaagcacttatagcttctgaagttgaaaaaatacctaacgaaaaggttaaatcaatagtaaaaaaacaccttaccgaattaaaagagggacaaagagattttagattctaaagtactgct

SEQ ID NO: 82 - DNA, 113 bp, Ralstonia eutropha
gatcttgtttcaactatgtcgccaagccagcattcgtgcgcgagggcggtatcgctccccggttggcgcatcgcgacgaatgccaataccaatacagaaattaggagacaggt

SEQ ID NO: 83 - DNA, 245 bp, Escherichia coli
tcaacactggcacaattattgctgtagctggcaatagttaatgggaggcgatatgcacgaaataaccctctgccaacgggcactggaattgatcgaacagcaggccgcaaaacacggcgcaaaacgcgtaactggggtctggctcaaaattggcgcattttcttgtgtcgaaaccagctctcttgccttttgttttgatctggtttgccgcggcagcgtggcggaaggttgtaaactgcacctcg